SEQUENCE LISTING (48 sequences)

SEQ ID NO: 1 - DNA, 1565 bp, Influenza A virus
  CDS (1)..(1494)
  misc_feature (1)..(1494): NP-gene from Influenza A virus Wilson-Smith/1933 (H1N1)
agcaaaagcagggtatataatcactcactgagtgacatccatatcatggcgtctcaaggcaccaaacggtcttatgagcagatggaaactggtggagagcgccagaatgctactgagatcagagcatctgttgggagaatggttagtggaatcggaagattctacatacagatgtgcactgaactcaagctcagcgactacgaaggaaggctgatccagaacagcataacaatagagagaatggttctctctgcatttgatgaaaggaggaataggtatctggaggaacatcccagtgctggaaaggaccctaagaaaactggaggtccaatctatagacggagagatggaaaatgggtgagagaactgatcttgtacgacaaagaggaaatcaggagaatttggcgccaagcgaacaatggagaggacgcaactgctggtctcacccatttgatgatctggcattccaatctgaatgatgccacgtatcagagaacaagagcccttgtgcgtactgggatggaccccagaatgtgctctctgatgcaaggttcaactctcccgaggagatctggagctgctggtgcagcggtgaagggagttgggacaatggtaatggaactgattcggatgataaagcggggaatgaatgaccggaatttctggagaggcgagaatggacggagaacaagaattgcttatgagagaatgtgcaacatcctcaaaggaaaatttcaaacagcagcacaacgagcaatgatggatcaggtgcgggaaagccggaatcctggaaatgctgaaattgaagacctcatcttcctggcacgatctgcactcatacttaggggatcagtggctcataagtcctgcctgcctgcttgtgtatatggacttactgtggccagtgggtacgactttgagagagaaggatactctctggtcggaatagatcctttccgtctgctccaaaacagccaggtattcagcctcatcagatcaaatgaaaatccagcacataagagtcaactggtatggatggcatgccattctgcagcatttgaagacctgagagtgtcaagcttcatcagggggacaagagtggtcccaagaggacaactacccaccagaggagtccaaattgcttcaaatgagaacatggagacaatggattcctccactcttgaactgaggagcagatactgggctataaggaccagaagcggaggaaacactaaccagcagagggcatctgcaggacaaatcagcgtacaacccactttctctgtacagagaaatctccctttcgagagagcgacaatcatggcagcatttacagggaacactgaaggcagaacgtcggacatgaggactgagatcataagaatgatggagagtgccagaccagaagatgtgtctttccaggggcggggagtcttcgagctctcggacgaaaaggcaacgagcccgatcgtgccttccttggacatgagtaatgagggatcttatttcttcggagacaatgcagaggagtatgacaattaaagaaaaatacccttgtttctact

SEQ ID NO: 2 - protein, 498 aa, Influenza A virus
SKSRVYNHSLSDIHIMASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGMNDRNFWRGENGRRTRIAYERMCNILKGKFQTAAQRAMMDQVRESRNPGNAEIEDLIFLARSALILRGSVAHKSCLPACVYGLTVASGYDFEREGYSLVGIDPFRLLQNSQVFSLIRSNENPAHKSQLVWMACHSAAFEDLRVSSFIRGTRVVPRGQLPTRGVQIASNENMETMDSSTLELRSRYWAIRTRSGGNTNQQRASAGQISVQPTFSVQRNLPFERATIMAAFTGNTEGRTSDMRTEIIRMMESARPEDVSFQGRGVFELSDEKATSPIVPSLDMSN

SEQ ID NO: 3 - DNA, 2151 bp, Influenza A virus
  CDS (1)..(2148)
  misc_feature (1)..(2148): PA-gene from Influenza A virus Puerto Rico/8/34 (H1N1)
atggaagattttgtgcgacaatgcttcaatccgatgattgtcgagcttgcggaaaaaacaatgaaagagtatggggaggacctgaaaatcgaaacaaacaaatttgcagcaatatgcactcacttggaagtatgcttcatgtattcagattttcacttcatcaatgagcaaggcgagtcaataatcgtagaacttggtgatccaaatgcacttttgaagcacagatttgaaataatcgagggaagagatcgcacaatggcctggacagtagtaaacagtatttgcaacactacaggggctgagaaaccaaagtttctaccagatttgtatgattacaaggagaatagattcatcgaaattggagtaacaaggagagaagttcacatatactatctggaaaaggccaataaaattaaatctgagaaaacacacatccacattttctcgttcactggggaagaaatggccacaaaggcagactacactctcgatgaagaaagcagggctaggatcaaaaccagactattcaccataagacaagaaatggccagcagaggcctctgggattcctttcgtcagtccgagagaggagaagagacaattgaagaaaggtttgaaatcacaggaacaatgcgcaagcttgccgaccaaagtctcccgccgaacttctccagccttgaaaattttagagcctatgtggatggattcgaaccgaacggctacattgagggcaagctgtctcaaatgtccaaagaagtaaatgctagaattgaaccttttttgaaaacaacaccacgaccacttagacttccgaatgggcctccctgttctcagcggtccaaattcctgctgatggatgccttaaaattaagcattgaggacccaagtcatgaaggagagggaataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaagactaaaaatatgaagaaaacaagtcagctaaagtgggcacttggtgagaacatggcaccagaaaaggtagactttgacgactgtaaagatgtaggtgatttgaagcaatatgatagtgatgaaccagaattgaggtcgcttgcaagttggattcagaatgagtttaacaaggcatgcgaactgacagattcaagctggatagagctcgatgagattggagaagatgtggctccaattgaacacattgcaagcatgagaaggaattatttcacatcagaggtgtctcactgcagagccacagaatacataatgaagggggtgtacatcaatactgccttgcttaatgcatcttgtgcagcaatggatgatttccaattaattccaatgataagcaagtgtagaactaaggagggaaggcgaaagaccaacttgtatggtttcatcataaaaggaagatcccacttaaggaatgacaccgacgtggtaaactttgtgagcatggagttttctctcactgacccaagacttgaaccacataaatgggagaagtactgtgttcttgagataggagatatgcttataagaagtgccataggccaggtttcaaggcccatgttcttgtatgtgagaacaaatggaacctcaaaaattaaaatgaaatggggaatggagatgaggcgttgcctcctccagtcacttcaacaaattgagagtatgattgaagctgagtcctctgtcaaagagaaagacatgaccaaagagttctttgagaacaaatcagaaacatggcccattggagagtcccccaaaggagtggaggaaagttccattgggaaggtctgcaggactttattagcaaagtcggtattcaacagcttgtatgcatctccacaactagaaggattttcagctgaatcaagaaaactgcttcttatcgttcaggctcttagggacaaccttgaacctgggacctttgatcttggggggctatatgaagcaattgaggagtgcctgattaatgatccctgggttttgcttaatgcttcttggttcaactccttccttacacatgcattgagttag

SEQ ID NO: 4 - protein, 716 aa, Influenza A virus
MEDFVRQCFNPMIVELAEKTMKEYGEDLKIETNKFAAICTHLEVCFMYSDFHFINEQGESIIVELGDPNALLKHRFEIIEGRDRTMAWTVVNSICNTTGAEKPKFLPDLYDYKENRFIEIGVTRREVHIYYLEKANKIKSEKTHIHIFSFTGEEMATKADYTLDEESRARIKTRLFTIRQEMASRGLWDSFRQSERGEETIEERFEITGTMRKLADQSLPPNFSSLENFRAYVDGFEPNGYIEGKLSQMSKEVNARIEPFLKTTPRPLRLPNGPPCSQRSKFLLMDALKLSIEDPSHEGEGIPLYDAIKCMRTFFGWKEPNVVKPHEKGINPNYLLSWKQVLAELQDIENEEKIPKTKNMKKTSQLKWALGENMAPEKVDFDDCKDVGDLKQYDSDEPELRSLASWIQNEFNKACELTDSSWIELDEIGEDVAPIEHIASMRRNYFTSEVSHCRATEYIMKGVYINTALLNASCAAMDDFQLIPMISKCRTKEGRRKTNLYGFIIKGRSHLRNDTDVVNFVSMEFSLTDPRLEPHKWEKYCVLEIGDMLIRSAIGQVSRPMFLYVRTNGTSKIKMKWGMEMRRCLLQSLQQIESMIEAESSVKEKDMTKEFFENKSETWPIGESPKGVEESSIGKVCRTLLAKSVFNSLYASPQLEGFSAESRKLLLIVQALRDNLEPGTFDLGGLYEAIEECLINDPWVLLNASWFNSFLTHALS

SEQ ID NO: 5 - DNA, 2259 bp, Influenza B virus
  CDS (1)..(2256)
  misc_feature (1)..(2256): PB1-gene from Influenza B virus (B/Lee/40)
atgaatataaatccatattttcttttcatagatgtacctatacaggcagcaatttcaacaacattcccatacaccggtgttcccccttattctcatggaacgggaacaggctacacaatagacaccgtgattagaacacacgagtactcaaacaagggaaaacaatacatttctgatgttacaggatgtgtaatggtagatccaacaaatgggccattacccgaagacaatgaaccgagtgcctatgcacaattggattgtgttctggaggctttggatagaatggatgaagaacatccaggtctgtttcaagcagggtcacagaatgccatggaggcactaatggtcacaacagtggacaaattgactcaggggagacagacctttgattggacggtgtgtagaaaccaacctgctgcaacggcactgaacacaacaataacctcttttaggttgaatgatttaaatggagccgacaagggtggattagtgcccttttgccaagatatcattgattcattagacaaacctgaaatgattttcttcacagtaaagaatataaagaaaaaattgcctgctaaaaacagaaagggtttccttataaaaagaatacctatgaaggtaaaagacagaataacaagagtggaatacatcaaaagagcattatcattaaacacaatgactaaagatgctgaaagaggcaaactaaaaagaagagcaattgccaccgctgggatacaaatcagaggatttgtattagtagttgaaaacttggctaaaaatatctgtgaaaatctagagcaaagtggtttacccgtaggtggaaacgaaaagaaggccaaactatcaaatgcagtggctaaaatgctcagtaattgtccaccaggagggatcagtatgactgtgacaggagacaatactaaatggaatgaatgcttaaatccaagaatctttttggctatgactgaaagaataaccagagacagcccaatttggttccgggatttttgtagtatagcaccggtcttgttctccaataaaatagctagattgggaaaagggttcatgataacaagtaaaacaaaaagactaaaagctcaaataccttgtcccgatctgtttaatataccattagaaagatataatgaagaaacaagggcaaaactgaaaaagctaaaacctttcttcaatgaagaaggaacggcatctctttcgccaggaatgatgatgggaatgtttaatatgctatctacagtattaggagtagccgcactagggataaaaaacattggaaacaaagaatacttatgggatggactgcagtcttcggatgattttgctctgtttgttaatgcaaaagatgaagagacatgtatggaaggaataaacgatttttaccgaacatgtaagctattgggaataaacatgagcaaaaagaaaagttactgtaatgaaactgggatgtttgaatttaccagcatgttttacagagatggatttgtatctaattttgcaatggaactcccttcatttggagtcgctggagtgaatgaatcagcagacatggcaataggaatgacaataataaagaacaatatgatcaacaatgggatgggcccagcaacggcacaaacagccatacaattattcatagctgactatagatacacctacaaatgccacaggggagattccaaagtggaagggaagagaatgaaaattataaaggagctatgggaaaacactaaaggaagagatggtctattagtagcagatggtgggcctaatctttacaatttgagaaacctgcatattccagaaataatattaaaatacaacataatggaccctgagtacaaaggacggttactgcatcctcaaaatccctttgtaggacatttgtctattgagggtatcaaagaagcagatataacacctgcacatggcccaataaagaaaatggactacgatgcggtatctggaactcatagttggagaaccaaaaggaacagatctatactaaacactgatcagaggaacatgattcttgaggaacaatgctacgctaagtgttgcaacctttttgaggcttgctttaacagtgcgtcatacaggaaaccagtaggccagcacagcatgcttgaagctatggcccacagattaagaatggatgcacgactggactatgagtcaggaaggatgtcaaaagaggatttcgaaaaagcaatggctcaccttggtgagattgggtacatgtaa

SEQ ID NO: 6 - protein, 752 aa, Influenza B virus
MNINPYFLFIDVPIQAAISTTFPYTGVPPYSHGTGTGYTIDTVIRTHEYSNKGKQYISDVTGCVMVDPTNGPLPEDNEPSAYAQLDCVLEALDRMDEEHPGLFQAGSQNAMEALMVTTVDKLTQGRQTFDWTVCRNQPAATALNTTITSFRLNDLNGADKGGLVPFCQDIIDSLDKPEMIFFTVKNIKKKLPAKNRKGFLIKRIPMKVKDRITRVEYIKRALSLNTMTKDAERGKLKRRAIATAGIQIRGFVLVVENLAKNICENLEQSGLPVGGNEKKAKLSNAVAKMLSNCPPGGISMTVTGDNTKWNECLNPRIFLAMTERITRDSPIWFRDFCSIAPVLFSNKIARLGKGFMITSKTKRLKAQIPCPDLFNIPLERYNEETRAKLKKLKPFFNEEGTASLSPGMMMGMFNMLSTVLGVAALGIKNIGNKEYLWDGLQSSDDFALFVNAKDEETCMEGINDFYRTCKLLGINMSKKKSYCNETGMFEFTSMFYRDGFVSNFAMELPSFGVAGVNESADMAIGMTIIKNNMINNGMGPATAQTAIQLFIADYRYTYKCHRGDSKVEGKRMKIIKELWENTKGRDGLLVADGGPNLYNLRNLHIPEIILKYNIMDPEYKGRLLHPQNPFVGHLSIEGIKEADITPAHGPIKKMDYDAVSGTHSWRTKRNRSILNTDQRNMILEEQCYAKCCNLFEACFNSASYRKPVGQHSMLEAMAHRLRMDARLDYESGRMSKEDFEKAMAHLGEIGYM

SEQ ID NO: 7 - DNA, 2313 bp, Influenza B virus
  CDS (1)..(2310)
  misc_feature (1)..(2310): PB2-gene from Influenza B virus (B/Lee/40)
atgacgttggctaaaattgaactactaaagcagctgttaagggacaatgaagccaaaacggtgttgagacagacaacggtagaccaatacaacataataagaaaattcaatacatcaagaattgaaaagaacccttcattaagaatgaagtgggccatgtgttccaattttcccttagctctgaccaagggtgatatggcaaatcgaatccccttggaatacaagggaatacaacttaaaacaaatgctgaagacataggaactaaaggacaaatgtgttcaatagcagcagttacctggtggaatacatatgggcccataggggatactgaagggtttgaaaaggtctacgaaagcttttttctcagaaagatgagacttgacaatgccacttggggccgaataacctttggccctgttgagagagtaagaaaaagagtactactaaacccgctcaccaaggaaatgcccccagatgaagcgagcaatgtaataatggaaatattattccctaaagaagcaggaataccaagagaatctacttggatacatagagaactgataaaagaaaaaagagaaaaattgaagggaacgatgataactcccattgtactggcatacatgcttgagagagaactagttgcccgaagaaggttcctgccagtagcaggagcaacatcagcagagttcatagaaatgctacattgcttacaaggtgaaaattggagacaaatatatcatccaggagggaataaactaactgaatctagatctcaatcaatgattgtagcttgcaggaagataatcagaagatcaatagttgcatcaaacccactagagctagctgtagagattgcaaataagactgtgatagacactgaacctttaaaatcatgtctggcagccctggatggaggtgatgtagcctgtgacataataagagctgcattaggattaaaaattagacaaagacaaagatttgggagacttgaactaaagagaatatcaggaagaggattcaaaaatgatgaagagatattaatcggaaacggaacaatacaaaagattggaatatgggacggagaagaggaattccatgtaagatgtggcgaatgcagggggatattgaaaaaaagccaaatgagaatggaaaaactactgataaattcagccaaaaaggaggacatgaaagatttaataatcttatgcatggtattttctcaagacactaggatgttccaaggagtgagaggagagataaattttcttaatcgagcaggccaacttttatcccccatgtaccaactccaacgatactttctgaataggagcaatgacctttttgatcaatggggatatgaggaatcacctaaagcaagtgagctacatgggataaatgaattaatgaatgcatctgactatacattgaaaggggttgtagtaacaaaaaatgtgattgatgattttagttctactgaaacagaaaaagtatctataacaaaaaatcttagtttaataaaaaggactggggaagttataatgggagccaatgacgtaagtgaattagaatcacaagcacagctaatgataacgtatgatacacccaagatgtgggaaatgggaacaaccaaagaactggtacaaaacacttaccaatgggtgcttaaaaatttagtaacattgaaggctcagtttcttttgggaaaagaagacatgttccaatgggatgcatttgaagcatttgaaagcataatccctcagaagatggctggtcagtacagtggatttgcaagagcagtgctcaaacaaatgagagaccaagaggttatgaaaactgaccaattcataaaattgttgcctttctgtttttcgccaccaaaattaaggagcaatggagagccttatcaatttttgaggcttatgctgaaaggaggaggggaaaatttcatcgaagtaaggaaagggtcccccttgttctcctacaatccacaaacggaaatcctaactatatgcggcagaatgatgtcattaaaaggaaaaattgaggatgaagaaagaaatagatcaatggggaatgcagtactggcaggctttcttgttagtggcaaatatgaccctgatcttggagatttcaaaaccattgaggaacttgaaagactaaaaccgggagaaaaagccaacatcttactttaccaaggaaagcccgttaaagtagttaaaaggaaaagatatagtgctttatccaatgatatttcacaagggattaagagacaaagaatgacagttgagtccatggggtgggccttgagctaa

SEQ ID NO: 8 - protein, 770 aa, Influenza B virus
MTLAKIELLKQLLRDNEAKTVLRQTTVDQYNIIRKFNTSRIEKNPSLRMKWAMCSNFPLALTKGDMANRIPLEYKGIQLKTNAEDIGTKGQMCSIAAVTWWNTYGPIGDTEGFEKVYESFFLRKMRLDNATWGRITFGPVERVRKRVLLNPLTKEMPPDEASNVIMEILFPKEAGIPRESTWIHRELIKEKREKLKGTMITPIVLAYMLERELVARRRFLPVAGATSAEFIEMLHCLQGENWRQIYHPGGNKLTESRSQSMIVACRKIIRRSIVASNPLELAVEIANKTVIDTEPLKSCLAALDGGDVACDIIRAALGLKIRQRQRFGRLELKRISGRGFKNDEEILIGNGTIQKIGIWDGEEEFHVRCGECRGILKKSQMRMEKLLINSAKKEDMKDLIILCMVFSQDTRMFQGVRGEINFLNRAGQLLSPMYQLQRYFLNRSNDLFDQWGYEESPKASELHGINELMNASDYTLKGVVVTKNVIDDFSSTETEKVSITKNLSLIKRTGEVIMGANDVSELESQAQLMITYDTPKMWEMGTTKELVQNTYQWVLKNLVTLKAQFLLGKEDMFQWDAFEAFESIIPQKMAGQYSGFARAVLKQMRDQEVMKTDQFIKLLPFCFSPPKLRSNGEPYQFLRLMLKGGGENFIEVRKGSPLFSYNPQTEILTICGRMMSLKGKIEDEERNRSMGNAVLAGFLVSGKYDPDLGDFKTIEELERLKPGEKANILLYQGKPVKVVKRKRYSALSNDISQGIKRQRMTVESMGWALS

SEQ ID NO: 9 - DNA, 2181 bp, Influenza B virus
  CDS (1)..(2178)
  misc_feature (1)..(2178): PA-gene from Influenza B virus (B/Lee/40)
atggatacttttattacaaagaatttccagactacaataatacaaaaggccaaaaacacaatggcagaatttagtgaagatcctgaattacagccagcagtactattcaacatctgcgtccatctggaggtctgctatgtaataagtgatatgaactttcttgatgaggaaggaaagacatatacagcattagaaggacaaggaaaagagcaaaatttgagaccacagtatgaagtgattgagggaatgccaagaaacatagcatggatggttcaaagatccttagcccaagagcatggaatagagactccaaggtatctggctgatttatttgattataaaaccaagaggtttatcgaagtcggaataacaaagggattggctgatgattacttttggaaaaagaaagaaaagttggggaatagcatggaactgatgatattcagctacaatcaagactactcgttaagtgatgaatcttcattggatgaggaaggaaaagggagagtgctaagcagactcacagaacttcaggctgagttaagtttgaaaaacctatggcaagttctaataggggaagaagaaattgaaaaaggaattgacttcaaacttggacaaacaatatctaaactgaggaatatatctgttccagctggtttctccaattttgaagggatgagaagttacatagacaacatagaccctaaaggagcaatagagagaaatctagcaaggatgtctcccttagtatcagttacacccaaaaagttgaaatgggaggacctgagacccatagggcctcacatttacaaccatgagctaccagaagttccatataatgcctttctcctcatgtctgatgagttggggctggccaatatgactgaaggaaagtccaagaaaccgaagaccttagctaaggaatgtctagaaaggtattcaacactacgtgatcaaactgacccaatattgataatgaaaagcgaaaaagctaacgaaaacttcttatggaggttatggagggactgtgtaaatacaataagcaatgaggaaacaggcaacgaattacagaaaaccaattatgccaagtgggccacaggagatggactaacataccaaaaaataatgaaagaagtagcaatagatgacgaaacgatgtaccaagaagaacccaaaatacccaataaatgtagagtggctgcttgggttcaggcagagatgaatctactgagtactctgacaagtaaaagggccctggatctgccagaaatagggccagatgtagcacccgtggagcatgtagggagtgaaagaaggaaatactttgttaatgaaatcaactactgtaaagcctctacagttatgatgaagtatgtactttttcacacttcattattaaatgaaagcaatgctagtatgggaaaatataaagtaataccaatcaccaacagagtggtaaatgaaaaaggggaaagctttgacatgctttatggtctggcggttaaggggcaatctcatttgcggggggacacggatgttgtaacagttgtgactttcgagtttagtagtacagatcctagagtggactcaggaaagtggccaaaatatactgtctttaaaattggctccctatttgtgagtggaagagaaaaacctgtgtacctatattgccgagtgaatggtacaaacaaaatccaaatgaaatggggaatggaagctagaagatgtctgcttcaatcaatgcaacaaatggaggcaattgttgatcaagaatcatcgatacaagggtatgatatgaccaaagcttgtttcaagggagacagagtgaataatcccaaaactttcagtattgggactcaggaaggcaaactagtaaaagggtcctttgggaaagcactaagagtaatattcaccaaatgtttgatgcattatgtatttggaaatgctcaattggaggggtttagtgccgaatctaggagacttctactgttaattcaggcattaaaagacaggaagggcccttgggtatttgacttggagggaatgtactttggagtagaggaatgtattagtaacaatccttgggtaatacagagtgcatactggtttaatgaatggttgggcattgaaaaagaaggaagtaaagtgttagaatcaatagatgaaataatggatgaatga

SEQ ID NO: 10 - protein, 726 aa, Influenza B virus
MDTFITKNFQTTIIQKAKNTMAEFSEDPELQPAVLFNICVHLEVCYVISDMNFLDEEGKTYTALEGQGKEQNLRPQYEVIEGMPRNIAWMVQRSLAQEHGIETPRYLADLFDYKTKRFIEVGITKGLADDYFWKKKEKLGNSMELMIFSYNQDYSLSDESSLDEEGKGRVLSRLTELQAELSLKNLWQVLIGEEEIEKGIDFKLGQTISKLRNISVPAGFSNFEGMRSYIDNIDPKGAIERNLARMSPLVSVTPKKLKWEDLRPIGPHIYNHELPEVPYNAFLLMSDELGLANMTEGKSKKPKTLAKECLERYSTLRDQTDPILIMKSEKANENFLWRLWRDCVNTISNEETGNELQKTNYAKWATGDGLTYQKIMKEVAIDDETMYQEEPKIPNKCRVAAWVQAEMNLLSTLTSKRALDLPEIGPDVAPVEHVGSERRKYFVNEINYCKASTVMMKYVLFHTSLLNESNASMGKYKVIPITNRVVNEKGESFDMLYGLAVKGQSHLRGDTDVVTVVTFEFSSTDPRVDSGKWPKYTVFKIGSLFVSGREKPVYLYCRVNGTNKIQMKWGMEARRCLLQSMQQMEAIVDQESSIQGYDMTKACFKGDRVNNPKTFSIGTQEGKLVKGSFGKALRVIFTKCLMHYVFGNAQLEGFSAESRRLLLLIQALKDRKGPWVFDLEGMYFGVEECISNNPWVIQSAYWFNEWLGIEKEGSKVLESIDEIMDE

SEQ ID NO: 11 - DNA, 1755 bp, Influenza B virus
  CDS (1)..(1752)
  misc_feature (1)..(1752): HA-gene from Influenza B virus (B/Lee/40)
atgaaggcaataattgtactactcatggtagtaacatccaatgcagatcgaatctgcactgggataacatcgtcaaactcacctcatgtggttaaaactgccactcaaggggaagtcaatgtgactggtgtgataccactaacaacaacacctaccaaatctcattttgcaaatctcaaaggaacacagaccagaggaaaactatgcccaaactgttttaactgcacagatctggacgtggccctaggcagaccaaaatgcatggggaacacaccctccgcaaaagtctcaatactccatgaagtcaaacctgctacatctggatgctttcctataatgcacgacagaacaaaaatcagacaactacctaatcttctcagaggatatgaaaacatcaggttatcaaccagtaatgttatcaatacagagacggcaccaggaggaccctacaaggtggggacctcaggatcttgccctaacgttgctaatgggaacggcttcttcaacacaatggcttgggttatcccaaaagacaacaacaagacagcaataaatccagtaacagtagaagtaccatacatttgttcagaaggggaagaccaaattactgtttgggggttccactctgatgacaaaacccaaatggaaagactctatggagactcaaatcctcaaaagttcacctcatctgccaatggagtaaccacacattatgtttctcagattggtggcttcccaaatcaaacagaagacgaagggctaaaacaaagcggcagaattgttgttgattacatggtacaaaaacctggaaaaacaggaacaattgtttatcaaagaggcattttattgcctcaaaaagtgtggtgcgcaagtggcaggagcaaggtaataaaagggtccttgcctttaattggtgaagcagattgcctccacgaaaagtacggtggattaaataaaagcaagccttactacacaggagagcatgcaaaggccataggaaattgcccaatatgggtgaaaacacccttgaagctggccaatggaaccaaatatagaccgcctgcaaaactattaaaggaaagaggtttcttcggagctattgctggtttcttggaaggaggatgggaaggaatgattgcaggttggcacggatacacatctcatggagcacatggagtggcagtggcagcagaccttaagagtacacaagaagctataaacaagataacaaaaaatctcaactatttaagtgagctagaagtaaaaaaccttcaaagactaagcggagcaatgaatgagcttcacgacgaaatactcgagctagacgaaaaagtggatgatctaagagctgatacaataagctcacaaatagagcttgcagtcttgctttccaacgaagggataataaacagtgaagatgagcatctcttggcacttgaaagaaaactgaagaaaatgcttggcccctctgctgtagaaatagggaatgggtgctttgaaaccaaacacaaatgcaaccagacttgcctagacaggatagctgctggcacctttaatgcaggagatttttctcttcccacttttgattcattaaacattactgctgcatctttaaatgatgatggcttggataatcatactatactgctctactactcaactgctgcttctagcttggctgtaacattaatgatagctatcttcattgtctacatggtctccagagacaatgtttcttgttccatctgtctgtga

SEQ ID NO: 12 - protein, 584 aa, Influenza B virus
MKAIIVLLMVVTSNADRICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHTILLYYSTAASSLAVTLMIAIFIVYMVSRDNVSCSICL

SEQ ID NO: 13 - DNA, 1683 bp, Influenza B virus
  CDS (1)..(1680)
  misc_feature (1)..(1680): NP-gene from Influenza B virus (B/Lee/40)
atgtccaacatggatattgacagtataaataccggaacaatcgataaaacaccagaagaactgactcccggaaccagtggggcaaccagaccaatcatcaagccagcaacccttgctccgccaagcaacaaacgaacccgaaatccatctccagaaaggacaaccacaagcagtgaaaccgatatcggaaggaaaatccaaaagaaacaaaccccaacagagataaagaagagcgtctacaaaatggtggtaaaactgggtgaattctacaaccagatgatggtcaaagctggacttaatgatgacatggaaaggaatctaattcaaaatgcacaagctgtggagagaatcctattggctgcaactgatgacaagaaaactgaataccaaaagaaaaggaatgccagagatgtcaaagaagggaaggaagaaatagaccacaacaagacaggaggcaccttttataagatggtaagagatgataaaaccatctacttcagccctataaaaattacctttttaaaagaagaggtgaaaacaatgtacaagaccaccatggggagtgatggtttcagtggactaaatcacattatgattggacattcacagatgaacgatgtctgtttccaaagatcaaagggactgaaaagggttggacttgacccttcattaatcagtacttttgccggaagcacactacccagaagatcaggtacaactggtgttgcaatcaaaggaggtggaactttagtggatgaagccatccgatttataggaagagcaatggcagacagagggctactgagagacatcaaggccaagacggcctatgaaaagattcttctgaatctgaaaaacaagtgctctgcgccgcaacaaaaggctctagttgatcaagtgatcggaagtaggaacccagggattgcagacatagaagacctaactctgcttgccagaagcatggtagttgtcagaccctctgtagcgagcaaagtggtgcttcccataagcatttatgctaaaatacctcaactaggattcaataccgaagaatactctatggttgggtatgaagccatggctctttataatatggcaacacctgtttccatattaagaatgggagatgacgcaaaagataaatctcaactattcttcatgtcgtgcttcggagctgcctatgaagatctaagagtgttatctgcactaacgggcaccgaatttaagcctagatcagcactaaaatgcaagggtttccatgtcccggctaaggagcaagtagaaggaatgggggcagctctgatgtccatcaagcttcagttctgggccccaatgaccagatctggagggaatgaagtaagtggagaaggagggtctggtcaaataagttgcagccctgtgtttgcagtagaaagacctattgctctaagcaagcaagctgtaagaagaatgctgtcaatgaacgttgaaggacgtgatgcagatgtcaaaggaaatctactcaaaatgatgaatgattcaatggcaaagaaaaccagtggaaatgctttcattgggaagaaaatgtttcaaatatcagacaaaaacaaagtcaatcccattgagattccaattaagcagaccatccccaatttcttctttgggagggacacagcagaggattatgatgacctcgattattaa

SEQ ID NO: 14 - protein, 560 aa, Influenza B virus
MSNMDIDSINTGTIDKTPEELTPGTSGATRPIIKPATLAPPSNKRTRNPSPERTTTSSETDIGRKIQKKQTPTEIKKSVYKMVVKLGEFYNQMMVKAGLNDDMERNLIQNAQAVERILLAATDDKKTEYQKKRNARDVKEGKEEIDHNKTGGTFYKMVRDDKTIYFSPIKITFLKEEVKTMYKTTMGSDGFSGLNHIMIGHSQMNDVCFQRSKGLKRVGLDPSLISTFAGSTLPRRSGTTGVAIKGGGTLVDEAIRFIGRAMADRGLLRDIKAKTAYEKILLNLKNKCSAPQQKALVDQVIGSRNPGIADIEDLTLLARSMVVVRPSVASKVVLPISIYAKIPQLGFNTEEYSMVGYEAMALYNMATPVSILRMGDDAKDKSQLFFMSCFGAAYEDLRVLSALTGTEFKPRSALKCKGFHVPAKEQVEGMGAALMSIKLQFWAPMTRSGGNEVSGEGGSGQISCSPVFAVERPIALSKQAVRRMLSMNVEGRDADVKGNLLKMMNDSMAKKTSGNAFIGKKMFQISDKNKVNPIEIPIKQTIPNFFFGRDTAEDYDDLDY

SEQ ID NO: 15 - DNA, 1401 bp, Influenza B virus
  CDS (1)..(1398)
  misc_feature (1)..(1398): NA-gene from Influenza B virus (B/Lee/40)
atgctaccttcaactgtacaaacattaaccctattactcacatcagggggagtattattatcactatatgtgtcagcctcattgtcatacttattgtattcggatgtattgctaaaattttcatcaacaaaaacaactgcaccaacaatgtcattagagtgcacaaacgcatcaaatgcccagactgtgaaccattctgcaacaaaagagatgacatttccacccccagagccggagtggacataccctcgtttatcttgccagggctcaacctttcagaaggcactcctaattagccctcataggttcggagagatcaaaggaaactcagctcccttgataataagagaaccttttgttgcttgtggaccaaaagaatgcagacactttgctctgacccattatgcagctcagccggggggatactacaatggaacaagaaaggacagaaacaagctgaggcatctagtatcagtcaaattgggaaaaatcccaactgtggaaaactccattttccacatggcagcttggagcggatccgcatgccatgatggtagagaatggacatatatcggagttgatggtcctgacaatgatgcattggtcaaaataaaatatggagaagcatatactgacacatatcattcctatgcacacaacatcctaagaacacaagaaagtgcctgcaattgcatcgggggagattgttatcttatgataacagacggctcagcttcaggaattagtaaatgcagatttcttaaaattagagagggtcgaataataaaagaaatacttccaacaggaagagtggagcacactgaagagtgcacatgcgggttcgccagcaataaaaccatagaatgtgcctgtagagacaacagttacacagcaaaaagaccctttgtcaaattaaatgtggaaactgatacagctgaaataagattgatgtgcacaaagacttatctagacactcccagaccggatgatggaagcatagcagggccttgcgaatctaatggagacaagtggcttggaggcatcaaaggaggattcgtccatcaaagaatggcatctaagattggaagatggtactcccgaacgatgtctaaaactaacagaatggggatggaactgtatgtaaagtatgatggtgacccatggactgacagtgatgctcttactcttagtggagtaatggtttccatagaagaacctggttggtattcttttggcttcgaaataaaggacaagaaatgtgatgtcccttgtattgggatagagatggtacacgatggtggaaaagatacttggcattcagctgcaacagccatttactgtttgatgggctcaggacaattgctatgggacactgtcacaggcgttgatatggctttataa

SEQ ID NO: 16 - protein, 466 aa, Influenza B virus
MLPSTVQTLTLLLTSGGVLLSLYVSASLSYLLYSDVLLKFSSTKTTAPTMSLECTNASNAQTVNHSATKEMTFPPPEPEWTYPRLSCQGSTFQKALLISPHRFGEIKGNSAPLIIREPFVACGPKECRHFALTHYAAQPGGYYNGTRKDRNKLRHLVSVKLGKIPTVENSIFHMAAWSGSACHDGREWTYIGVDGPDNDALVKIKYGEAYTDTYHSYAHNILRTQESACNCIGGDCYLMITDGSASGISKCRFLKIREGRIIKEILPTGRVEHTEECTCGFASNKTIECACRDNSYTAKRPFVKLNVETDTAEIRLMCTKTYLDTPRPDDGSIAGPCESNGDKWLGGIKGGFVHQRMASKIGRWYSRTMSKTNRMGMELYVKYDGDPWTDSDALTLSGVMVSIEEPGWYSFGFEIKDKKCDVPCIGIEMVHDGGKDTWHSAATAIYCLMGSGQLLWDTVTGVDMAL

SEQ ID NO: 17 - DNA, 747 bp, Influenza B virus
  CDS (1)..(744)
  misc_feature (1)..(744): M-gene from Influenza B virus (B/Lee/40)
atgtcgctgtttggagacacaattgcctacctgctttcactaatagaagatggagaaggcaaagcagaactagctgaaaaattacactgttggttcggtgggaaagaatttgacctagattctgctttggaatggataaaaaacaaaaggtgcctaactgatatacaaaaagcactaattggtgcctctatatgctttttaaaacccaaagaccaagaaagaaaaaggagattcatcacagagcccctgtcaggaatgggaacaacagcaacaaagaagaaaggcctaattctagctgagagaaaaatgagaagatgtgtaagctttcatgaagcatttgaaatagcagaaggccacgaaagctcagcattactatattgtcttatggtcatgtacctaaaccctgaaaactattcaatgcaagtaaaactaggaacgctctgtgctttatgcgagaaacaagcatcgcactcgcatagagcccatagcagagcagcaaggtcttcggtacctggagtaagacgagaaatgcagatggtttcagctatgaacacagcaaagacaatgaatggaatgggaaagggagaagacgtccaaaaactagcagaagagctgcaaaacaacattggagtgttgagatctctaggagcaagtcaaaagaatggagaaggaattgccaaagatgtaatggaagtgctaaaacagagctctatgggaaattcagctcttgtgaggaaatacttataa

SEQ ID NO: 18 - protein, 248 aa, Influenza B virus
MSLFGDTIAYLLSLIEDGEGKAELAEKLHCWFGGKEFDLDSALEWIKNKRCLTDIQKALIGASICFLKPKDQERKRRFITEPLSGMGTTATKKKGLILAERKMRRCVSFHEAFEIAEGHESSALLYCLMVMYLNPENYSMQVKLGTLCALCEKQASHSHRAHSRAARSSVPGVRREMQMVSAMNTAKTMNGMGKGEDVQKLAEELQNNIGVLRSLGASQKNGEGIAKDVMEVLKQSSMGNSALVRKYL

SEQ ID NO: 19 - DNA, 846 bp, Influenza B virus
  CDS (1)..(843)
  misc_feature (1)..(843): NS-gene from Influenza B virus (B/Lee/40)
atggcggacaacatgaccacaacacaaattgaggtgggtccgggagcaaccaatgccactataaactttgaagcaggaattctggagtgctatgaaaggttttcatggcaaagagcccttgactatcctggtcaagaccgcctacacagactaaaacgaaaattagaatcaagaataaagactcacaacaagagtgagcctgagaataaaaggatgtctcttgaagagagaaaagcaattggggtaaaaatgatgaaagtgcttctgtttatggatccctctgctggaattgaagggtttgagccatactgtgtgaaaaatccctcaactagcaaatgtccaaattacgattggaccgattaccctccaaccccaggaaagtaccttgatgacatagaagaagagccggaaaatgtcgatcacccaattgaggtagtattaagggacatgaacaataaagatgcacgacaaaagataaaggatgaagtaaacactcagaaagaggggaaattccgtttgacaataaaaagggatatacgtaatgtgttgtccttgagagtgttggtgaacggaaccttcctcaagcaccctaatggagacaagtccttatcaactcttcatagattgaatgcatatgaccagaatggagggcttgttgctaaacttgttgctactgatgatcggacagtggaggatgaaaaagatggccatcggatcctcaactcactcttcgagcgttttgatgaaggacattcaaagccaattcgagcagctgaaactgcggtgggagtcttatcccaatttggtcaagagcaccgattatcaccagaagagggagacaattag

SEQ ID NO: 20 - protein, 281 aa, Influenza B virus
MADNMTTTQIEVGPGATNATINFEAGILECYERFSWQRALDYPGQDRLHRLKRKLESRIKTHNKSEPENKRMSLEERKAIGVKMMKVLLFMDPSAGIEGFEPYCVKNPSTSKCPNYDWTDYPPTPGKYLDDIEEEPENVDHPIEVVLRDMNNKDARQKIKDEVNTQKEGKFRLTIKRDIRNVLSLRVLVNGTFLKHPNGDKSLSTLHRLNAYDQNGGLVAKLVATDDRTVEDEKDGHRILNSLFERFDEGHSKPIRAAETAVGVLSQFGQEHRLSPEEGDN

SEQ ID NO: 21 - DNA, 330 bp, Influenza B virus
  CDS (1)..(327)
  misc_feature (1)..(327): BM2-gene from Influenza B virus (B/Lee/40)
atgctcgaaccacttcagattctttcaatttgttctttcattttatcagctctccatttcatggcttggacaatagggcatttgaatcaaataagaagaggggtaaacctgaaaatacaaataaggaatccaaataaggaggcaataaacagagaggtgtcaattctgagacacaattaccaaaaggaaatccaagccaaagaaacaatgaagaaaatactctctgacaacatggaagtattgggtgaccacatagtagttgaagggctttcaactgatgagataataaaaatgggtgaaacagttttggaggtggaagaattgcaatga

SEQ ID NO: 22 - protein, 109 aa, Influenza B virus
MLEPLQILSICSFILSALHFMAWTIGHLNQIRRGVNLKIQIRNPNKEAINREVSILRHNYQKEIQAKETMKKILSDNMEVLGDHIVVEGLSTDEIIKMGETVLEVEELQ

SEQ ID NO: 23 - DNA, 369 bp, Influenza B virus
  CDS (1)..(366)
  misc (1)..(366): NS-2 gene from Influenza B virus (B/Lee(40)
atggcggacaacatgaccacaacacaaattgagtggaggatgaaaaagatggccatcggatcctcaactcactcttcgagcgttttgatgaaggacattcaaagccaattcgagcagctgaaactgcggtgggagtcttatcccaatttggtcaagagcaccgattatcaccagaagagggagacaattagactggccacggaagaactttatctcttgagtaaaagaattgatgatagtatattgttccacaaaacagtaatagctaacagctccataatagctgacatgattgtatcattatcattactggaaacattgtatgaaatgaaggatgtggttgaagtgtacagcaggcagtgcttatga

SEQ ID NO: 24 - protein, 122 aa, Influenza B virus
MADNMTTTQIEWRMKKMAIGSSTHSSSVLMKDIQSQFEQLKLRWESYPNLVKSTDYHQKRETIRLATEELYLLSKRIDDSILFHKTVIANSSIIADMIVSLSLLETLYEMKDVVEVYSRQCL

SEQ ID NO: 25 - DNA, 27 bp, Artificial
  primer are used for the site-directed mutations of NP gene of WSN/33 virus
gtaatgaaggatcctatttcttcggag

SEQ ID NO: 26 - DNA, 27 bp, artificial
  primer are used for the site-directed mutations of NP gene of WSN/33 virus
ctccgaagaaataggatccttcattac

SEQ ID NO: 27 - DNA, 27 bp, artificial
  primer are used for the site-directed mutations of NP gene of WSN/33 virus
gatcctatttctttggagacaatgcag

SEQ ID NO: 28 - DNA, 27 bp, artificial
  primer are used for the site-directed mutations of NP gene of WSN/33 virus
ctgcattgtctccaaagaaataggatc

SEQ ID NO: 29 - DNA, 25 bp, artificial
  primer are used for the site-directed mutations of NP gene of WSN/33 virus
tttctttggagataatgcagaggag

SEQ ID NO: 30 - DNA, 25 bp, artificial
  primer are used for the site-directed mutations of NP gene of WSN/33 virus
ctcctctgcattatctccaaagaaa

SEQ ID NO: 31 - DNA, 27 bp, artificial
  primer are used for the site-directed mutations of NP gene of WSN/33 virus
tgagtaatgaaggctcctatttctttg

SEQ ID NO: 32 - DNA, 27 bp, artificial
  primer are used for the site-directed mutations of NP gene of WSN/33 virus
caaagaaataggagccttcattactca

SEQ ID NO: 33 - DNA, 25 bp, artificial
  primer are used for the site-directed mutations of NP gene of WSN/33 virus
ctatttctttggcgataatgcagag

SEQ ID NO: 34 - DNA, 25 bp, artificial
  primer are used for the site-directed mutations of NP gene of WSN/33 virus
ctctgcattatcgccaaagaaatag

SEQ ID NO: 35 - DNA, 23 bp, artificial
  primer are used for the site-directed mutations of NP gene of WSN/33 virus
tttggcgataacgcagaggagta

SEQ ID NO: 36 - DNA, 23 bp, artificial
  primer are used for the site-directed mutations of NP gene of WSN/33 virus
tactcctctgcgttatcgccaaa

SEQ ID NO: 37 - DNA, 41 bp, artificial
  primer are used for the site-directed mutations of NP gene of WSN/33 virus
tatttctttggcgataacgccgaggagtacgacaattaaag

SEQ ID NO: 38 - DNA, 41 bp, artificial
  primer are used for the site-directed mutations of NP gene of WSN/33 virus
ctttaattgtcgtactcctcggcgttatcgccaaagaaata

SEQ ID NO: 39 - DNA, 45 bp, artificial
  primer are used for the site-directed mutations of NP gene of WSN/33 virus
aaaaggcaacgagcccaatcgtaccctcctttgacatgagtaatg

SEQ ID NO: 40 - DNA, 45 bp, artificial
  primer are used for the site-directed mutations of NP gene of WSN/33 virus
cattactcatgtcaaaggagggtacgattgggctcgttgcctttt

SEQ ID NO: 41 - DNA, 20 bp, artificial
  TaqMan sense primer for NP gene
gcggggaaagatcctaagaa

SEQ ID NO: 42 - DNA, 23 bp, artificial
  TaqMan antisense primer for NP gene
tccactttccatctactctcctg

SEQ ID NO: 43 - DNA, 20 bp, artificial
  TaqMan sense primer for M gene
cctggtatgtgcaacctgtg

SEQ ID NO: 44 - DNA, 21 bp, artificial
  TaqMan antisense primer for M gene
tgtcaccatttgcctatgaga

SEQ ID NO: 45 - DNA, 21 bp, artificial
  TaqMan sense primer for PA gene
ctgacccaagacttgaaccac

SEQ ID NO: 46 - DNA, 26 bp, artificial
  TaqMan antisense primer for PA gene
agcatatctcctatctcaagaacaca

SEQ ID NO: 47 - DNA, 1497 bp, Influenza A virus
  CDS (1)..(1494)
  misc (1)..(1497): NP-gene from A/WSN33
atggcgaccaaaggcaccaaacgatcttacgaacagatggagactgatggagaacgccagaatgccactgaaatcagagcatctgtcggaaaaatgattgatggaattggacgattctacatccaaatgtgcaccgaacttaaactcagtgattatgagggacggctgattcagaacagcttaacaatagagagaatggtgctctctgcttttgacgagaggaggaataaatatctagaagaacatcccagtgcggggaaagatcctaagaaaactggaggacctatatacaggagagtagatggaaagtggaggagagaactcatcctttatgacaaagaagaaataagacgaatctggcgccaagctaataatggtgacgatgcaacggctggtctgactcacatgatgatctggcactccaatttgaatgatgcaacttaccagaggacaagagctcttgttcgcacaggaatggatcccaggatgtgctcactgatgcagggttcaaccctccctaggaggtctggggccgcaggtgctgcagtcaaaggagttggaacaatggtgatggaattgatcagaatgatcaaacgtgggatcaatgatcggaacttctggaggggtgagaatggacggagaacaaggattgcttatgaaagaatgtgcaacattctcaaagggaaatttcaaacagctgcacaaagaacaatggtggatcaagtgagagagagccggaatccaggaaatgctgagttcgaagatctcatctttttagcacggtctgcactcatattgagagggtcagttgctcacaagtcctgcctgcctgcctgtgtgtatggatctgccgtagccagtggatacgactttgaaagagagggatactctctagtcggaatagaccctttcagactgcttcaaaacagccaagtatacagcctaatcagaccaaatgagaatccagcacacaagagtcaactggtgtggatggcatgccattctgctgcatttgaagatctaagagtatcaagcttcatcagagggacgaaagtggtccccagagggaagctttccactagaggagttcaaattgcttccaatgaaaacatggagactatggaatcaagtacccttgaactgagaagcagatactgggccataaggaccagaagtggagggaacaccaatcaacagagggcttcctcgggccaaatcagcatacaaccgacgttctcagtacagagaaatctcccatttgacagaccaaccattatggcagcattcactgggaatacagaggggagaacatctgacatgagaaccgaaatcataaggctgatggaaagtgcaagacccgaagatgtgtctttccaggggcggggagtcttcgagctctcggacgaaaaggcaacgagcccaatcgtgccatcctttgacatgagtaatgaaggatcttatttcttcggagacaatgcagaggagtacgacaattaa

SEQ ID NO: 48 - protein, 498 aa, Influenza A virus
MATKGTKRSYEQMETDGERQNATEIRASVGKMIDGIGRFYIQMCTELKLSDYEGRLIQNSLTIERMVLSAFDERRNKYLEEHPSAGKDPKKTGGPIYRRVDGKWRRELILYDKEEIRRIWRQANNGDDATAGLTHMMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRTMVDQVRESRNPGNAEFEDLIFLARSALILRGSVAHKSCLPACVYGSAVASGYDFEREGYSLVGIDPFRLLQNSQVYSLIRPNENPAHKSQLVWMACHSAAFEDLRVSSFIRGTKVVPRGKLSTRGVQIASNENMETMESSTLELRSRYWAIRTRSGGNTNQQRASSGQISIQPTFSVQRNLPFDRPTIMAAFTGNTEGRTSDMRTEIIRLMESARPEDVSFQGRGVFELSDEKATSPIVPSFDMSNEGSYFFGDNAEEYDN

(19) United States
(12) Patent Application Publication
(10) Pub. No.: US 2014/0302077 A1
(43) Pub. Date: Oct. 9, 2014

(54) LIVE ATTENUATED INFLUENZA VIRUS

(71) Applicants: Darisuren Anhlan, Muenster (DE); Christoph Scholtissek, Linden (DE); Stephan Ludwig, Muenster (DE)

(72) Inventors: Darisuren Anhlan, Muenster (DE); Christoph Scholtissek, Linden (DE); Stephan Ludwig, Muenster (DE)

(73) Assignee: Westfaelische Wilhelms-Universitaet Munester, Munester (DE)

(21) Appl. No.: 14/241,165

(22) PCT Filed: Aug. 28, 2012
(86) PCT No.: PCT/EP2012/066655

§ 371 Date: May 21, 2014

(30) Foreign Application Priority Data
Sep. 2, 2011 (EP) 11179820.3

(51) Int. Cl. C12N 7/00; C12Q 1/70

(52) U.S. Cl. CPC C12N 7/00; C12Q 1/701
USPC 424/186.1; 435/5; 435/236; 536/23.72; 435/320.1; 435/350

(57) ABSTRACT

Provided are live attenuated influenza A and B viruses as well as a composition, influenza A and B genes, a vector with a respective gene, a host cell comprising such vector, a method for preparing a live attenuated influenza A or B virus and a use of the influenza viruses as a vaccine. An influenza A virus contains a NP-gene, which includes a silent mutation at one or more positions selected from nucleotide 1467, nucleotide 1473, nucleotide 1500, nucleotide 1503, nucleotide 1512, nucleotide 1515, nucleotide 1518, nucleotide 1521, and nucleotide 1524 of SEQ ID No: 1. A PA gene includes a silent mutation at one or more positions corresponding to a position selected from nucleotides 2100 and 2103 of SEQ ID No: 3.

### FIELD OF THE INVENTION

The present invention relates to methods for obtaining a live attenuated influenza virus by introducing one or more synonymous mutations within RNA packaging signals of an influenza virus gene segment as well as influenza viruses obtainable by said methods. Provided are also compositions with a live, attenuated influenza virus, a use thereof as well as influenza genes comprising one or more synonymous mutations within RNA packaging signals of an influenza virus gene segment. Provided is further a method for preparing a live, attenuated influenza virus. Also provided is a live, attenuated influenza virus for use in the vaccination against influenza. The attenuated influenza virus is an attenuated influenza A virus (IAV), an attenuated influenza B virus (IBV) or attenuated influenza C virus (ICV).

### BACKGROUND OF THE INVENTION

Human influenza (human flu) is a highly contagious respiratory disease typically starting with an abrupt onset of fever, sore throat, blocked or running nose, headache, photophobia, dry cough and malaise. It gives rise to repeating and frequent epidemics and pandemics that occur suddenly, causing substantial morbidity and mortality. The first recorded influenza pandemic dates back to 1580. Over the course of history there have been several influenza pandemics that have sickened and killed millions. Most cases of death have been found to be a result of an increased physiologic load in an already compromised host, or to be the outcome of the combined effects of the viral disease and a secondary bacterial infection. The 1918 influenza virus, called the “Spanish flu”, was particularly lethal, accounting for more than 40 million deaths worldwide. Albeit this strain caused pneumonia, also in this pandemic most deaths were associated with secondary bacterial pathogens.

Over the past two decades, the human toll from influenza in the United States alone has averaged 200,000 hospitalizations and 36,000 deaths per year. The first influenza pandemic of the 21st century was caused by Influenza A H1N1 (2009), a novel H1N1 subtype of influenza A virus first identified in Mexico and the United States in March and April 2009, respectively. As of February 2010, more than 212 countries and overseas territories or communities have reported laboratory-confirmed cases of pandemic influenza, including more than 15,000 deaths.

Influenza viruses are RNA viruses that replicate their genome in the nucleus of the host cell. They belong to the family Orthomyxoviridae and are divided into three genera A, B and C, which can be distinguished by antigenic differences in two of the structural proteins of the virus, the matrix protein M2 and the nucleoprotein. Each of these types has many strains. These are enveloped viruses with a segmented genome containing seven or eight single-stranded segments of negative-sense RNA. Each of these RNA segments contains one or two genes. The genomes of influenza A and influenza B virus consist of eight RNA segments, which are coding for 12 viral proteins (Steinhauer, D. A. & Skehel, J. J., Ann. Rev. Genet. (2002), 36, 305-332; Hutchinson, E. C., et al., Journal of General Virology (2010) 91, 313-328). The three largest gene segments of influenza A virus encode the subunits of the viral polymerase, PB2, PB1, and PA. The fourth segment encodes the hemagglutinin glycoprotein (HA), responsible for binding to cell-surface receptors and membrane fusion, and the fifth gene segment encodes the nucleoprotein (NP), which encapsidates cRNAs and vRNAs, which allows them to be recognized as templates for the viral polymerase. Segment 6 encodes the neuraminidase (NA), which cleaves sialic acid from virus and host cell glycoconjugates to allow mature virus particles to be released. The seventh segment generates two gene products, the matrix protein, M1, and the M2 transmembrane protein, which has proton channel activity. In influenza B virus this segment encodes matrix protein M1 and BM2, thought to be a functional counterpart of M2. The eighth gene segment encodes the protein NS1, which inter alia sequesters ds RNA formed during virus replication, and the nuclear export protein (NEP). To produce an intact virion or infectious influenza A virus an effective incorporation of all 8 gene segments into viral particle is necessary.

Influenza B and C viruses can infect only humans, although there have been reports of influenza B virus isolation from seals and influenza C virus isolation from pigs. In contrast thereto Influenza A viruses can infect both mammals and birds. The most devastating flu viruses of the 20th century, the Spanish flu pandemic in 1918 (H1N1), the Asian flu pandemic in 1957 (H2N2) and the Hong Kong flu pandemic in 1968 (H3N2), were all of avian origin. Aquatic birds are natural reservoirs of influenza A viruses. These viruses are known to cross the species barrier and cause either transitory infections or establish permanent lineages in mammals including man. While influenza B viruses do not have pandemic potential, they cause significant disease and are the predominant circulating strain of influenza virus approximately one in every 3 years. Influenza B virus is therefore an essential component of the influenza vaccine administered to susceptible groups such as the elderly and asthmatic.

Approved influenza vaccines are available since World War II, in the form of inactIAVted virus from infected embryonated eggs for injection. Such a seasonal vaccine contains three influenza viruses, a strain each of H3N2, H1N1 influenza A virus and an influenza B virus, either as a whole, chemically disrupted or in the form of isolated surface glycoproteins.

However, parenteral vaccination provides only limited protection. It is not effective at eliciting local IgA production, if there has been no previous mucosal exposure. An alternative form of vaccination is therefore a topical application to a mucosal surface. This administration route has the advantage of involving respiratory IgA for protection, since both secretory IgA and serum IgG have been shown to participate in immunity to influenza virus. A further advantage of stimulating a local IgA response to influenza is that it is often of a broader specificity than the serum response and can thus provide cross-protection against viruses possessing hemagglutinin molecules different from those present in the vaccine. However, inactivated vaccines are often poorly immunogenic when given mucosally. In this regard during the 1960s in the USSR and the US cold-adapted and attenuated live influenza virus vaccines were developed by reassortment of the six internal genes of the influenza viruses with the two surface genes of wild-type virus. A cold-adapted virus can replicate efficiently at 25° C. in the nasal passages, which are below normal body temperature. The virus has also been shown to be temperature sensitive in that its replication is impeded at the higher temperatures of the lungs. Therefore such a live attenuated virus has been used to stimulate the mucosal immune system.

Hence besides allowing intranasal administration, which is the natural route of infection, a live cold-adapted reassortant influenza vaccine allows induction of both local and humoral immunity and provides the possibility of application in the form of a single dose. The first FDA approved intranasal spray vaccine, Flumist™, was developed at the University of Michigan School of Public Health, and by MedImmune LLC, approved and recommended for seasonal influenza. A further intranasal spray vaccine, against influenza A (H1N1) 2009, by MedImmune LLC has been approved by the FDA. Both vaccines are cold-adapted live attenuated influenza viruses. The replication of such a cold-adapted virus is only slightly restricted in the cooler upper respiratory tract, but highly restricted in the warmer lower respiratory tract, the major site of disease-associated pathology. Both vaccines are approved for healthy children 24 months of age and older, adolescents, and healthy adults, up to 49 years of age. The two vaccines are not licensed for use in “at-risk” populations. Besides limitations in amount of doses that can be manufactured each year, the vaccines are not licensed for use in elderly populations, which are in particular need of protection from influenza. Therefore there remains a need for an alternative virus that can be applied intranasally and that is not restricted to the particular virus strains of a certain season.

It is thus an object of the present invention to provide an influenza virus that when used as a vaccine overcomes at least some of the above draw backs.

### SUMMARY OF THE INVENTION

The present invention provides modified attenuated influenza viruses that may be employed as an influenza virus vaccine. A modified virus according to the invention may also be a recombinant attenuated influenza virus suitable for use as a viral vector for expression of heterologous sequences in target cells.

In a first aspect, the present invention provides a method for obtaining a live, attenuated live influenza virus, said method comprising

(a) comparing a plurality of nucleotide sequences of RNA packaging signals of a gene segment of an influenza virus;
(b) identifying (a) conserved nucleotide(s) at the third position of a codon;
(c) substituting said conserved nucleotide(s) by (a) synonymous nucleotide(s) (i.e., introducing a synonymous mutation);
(d) producing an influenza virus comprising said synonymous nucleotide(s);
(e) determining whether an influenza virus containing said synonymous nucleotide(s) at the position(s) corresponding to the respective position(s) within the RNA packaging signal of an influenza virus not containing said synonymous nucleotide(s) is attenuated in comparison to the same influenza virus not containing said synonymous nucleotide(s) within the respective RNA packaging signal; and
(f) obtaining said attenuated influenza virus.

In a second aspect the present invention provides a live, attenuated influenza virus obtainable by the method of the first aspect of the invention. Said attenuated influenza virus can be an influenza A virus (IAV), influenza B virus (IBV) or influenza C virus (ICV).

In a third aspect the present invention provides a composition. The composition includes a live, attenuated influenza virus, preferably an influenza A virus (IAV). Said attenuated influenza virus is preferably obtainable by the method of the first aspect of the invention. The IAV contains a NP-gene, which includes a silent mutation at one or more positions. These positions correspond to a position selected from nucleotide 1467 (NP-A7), nucleotide 1473 (NP-A8), nucleotide 1500 (NP-A3), nucleotide 1503 (NP-A), nucleotide 1512 (NP-A1), nucleotide 1515 (NP-A4), nucleotide 1518 (NP-A2), nucleotide 1521 (NP-A5), and nucleotide 1524 (NP-A6) of SEQ ID No: 1. The composition also contains a pharmaceutically acceptable carrier.

In a fourth aspect the present invention provides an IAV NP-gene. The NP gene includes a silent mutation at one or more positions corresponding to a position selected from NP-A7, NP-A8, NP-A3, NP-A, NP-A1, NP-A4, NP-A2, NP-A5 and NP-A6 of the nucleotide sequence shown in SEQ ID NO: 1. Position NP-A7 is nucleotide 1467 of SEQ ID NO: 1, position NP-A8 is nucleotide 1473 of SEQ ID NO: 1, position NP-A3 is nucleotide 1500 of SEQ ID NO: 1, position NP-A is nucleotide 1503 of SEQ ID NO: 1, position NP-A1 is nucleotide 1512 of SEQ ID NO: 1, position NP-A4 is nucleotide 1515 of SEQ ID NO: 1, position NP-A2 is nucleotide 1518 of SEQ ID NO: 1, position NP-A5 is nucleotide 1521 of SEQ ID NO: 1, and position NP-A6 is nucleotide 1524 of SEQ ID No: 1.

In a fifth aspect the present invention provides an IAV PA-gene. The PA gene includes a silent mutation at one or more positions corresponding to a position selected from PA-A1 and PA-A2 of the nucleotide sequence shown in SEQ ID NO: 1. Position PA-A1 is nucleotide 2100 of SEQ ID No: 3. Position PA-A2 is nucleotide 2103 of SEQ ID No: 3.

In a sixth aspect the present invention provides a host cell. The host cell includes a vector, which vector includes the NP and/or PA gene.

In a seventh aspect the invention provides a method for the preparation of a live, attenuated IAV. The method includes introducing a vector into a host cell. The vector includes the PA-gene according to the second aspect. The method further includes introducing a plurality of vectors into the host cell. The plurality of vectors includes the remaining IAV genes required to form an infectious IAV. The method also includes isolating infectious IAV from the host cell.

In an eighth aspect the present invention provides a method for the preparation of a live, attenuated IAV. The method includes culturing the host cell according to the third aspect. The method further includes isolating infectious IAV from the host cell.

In an ninth aspect the invention provides a live, attenuated IAV. The live attenuated IAV includes a PA polymerase subunit encoded by the IAV PA gene according to the second aspect. In some embodiments the live, attenuated IAV is obtainable by a method according to the fourth or the fifth aspect.

In a tenth aspect the present invention provides a vaccine composition. The vaccine composition includes a live, attenuated IAV according to the sixth aspect. The vaccine composition further includes a pharmaceutically acceptable carrier.

In an eleventh aspect the present invention provides the IAV as described herein for use in the prevention and/or treatment of influenza.

In a twelfth aspect the present invention provides the IAV as described herein for use in the vaccination against influenza.

### BRIEF DESCRIPTION OF THE DRAWINGS

The invention will be better understood with reference to the detailed description when considered in conjunction with the non-limiting examples and the accompanying drawings.

FIG. 1: Schematic summary of identified packaging signal regions of 8 RNA segments of IAV. The entire packaging signal of each RNA segment consists of universal untranslated regions (UTR), which are comprised of completely conserved regions, both at the 5″end, (13 nucleotides) and 3″end (12 nucleotides), and the non-conserved segment specific regions of vRNA. Brighter grey background colour represents whole packaging signal regions in both ends of the open reading frame (ORF). According to references (see above), packaging signals in the ORF (amount of base nucleotides in chamber) depend on the each gene segment (see also Liang et al. (2005), J Virol 79:10348-10355; Wit et al. (2006), Vaccine 24:6647-6450, Gog et al. (2007), Nucleic Acids Res 35:1897-1907, Marsh et al. (2007), J Virol 81:9727-9236, Ozawa et al. (2007), J Virol 81:30-41, Hutchinson et al. (2009), J Gen Virol 91(Pt 2):313-328, Fujii et al. (2003), Proc Natl Acad Sci USA 100:2002-2007, Hutchinson et al. (2008), J Virol 82:11869-11879, Fujii et al. (2005), J Virol 79:3766-3674 (all incorporated by reference).

FIG. 2a: Introduced silent mutations for NP genes. Adenine (A), cytosine (C) or Thymine (T) changed nucleotides in each silent mutated NP gene construct (A—one nucleotide (C), A1—two nucleotides (C) and (T), A2—three nucleotides (C), (T), and (T), A3-four nucleotides (C), (T), (T), and (C), A4—five nucleotides (C), (T), (T), (C), and (C), A5—six nucleotides (C), (T), (T), (C), (C), and (C), A6—seven nucleotides (C), (T), (T), (C), (C), (C), and (C), and A8—nine nucleotides (C), (T), (T), (C), (C), (C), (C), (A), and (A) respectively.

FIG. 2b: Virus replication kinetics on MDCK cells. MDCK cells were infected with the WSN-WT virus or the depicted WSN-NP mutant strains ((B) MOI=0.01 or (C) MOI=0.001). 8 h, 24 h, and 48 h upon infection, supernatants were collected and virus titers were determined by plaque assay.

FIG. 2c: Virus titers were determined as described in the legend for FIG. 2b.

FIG. 2d: Virus plaque morphology on MDCK cells. Standard plaque assay was conducted with the WSN-WT, and WSN-A8 virus. 72 h upon plaque assay, plaque size morphology on MDCK cells was determined by staining of the MDCK cell layer and taking of photographs.

FIG. 3a: Mice body weight loss curve. Body weight of BALB/c mice was measured every day after intranasal (i.n.) inoculation with 1×10e6 pfu of WSN-WT and WSN-A8 mutant viruses. PBS infected mice were used as mock group mice. The average weight curve (in total %) with standard deviations of 5 mice in each group are depicted.

FIG. 3b: Survival curve. Survival analysis was performed under the same experiment conditions as described in the legend for FIG. 3a. All mice, which were infected with the mutant virus containing silent mutated NP gene survived in contrast to the overall letal WSN-WT virus infection.

FIG. 4a: Homolog immunity and cross-protection after 45 days post immunization with the attenuated WSN-A8 virus (Survival curve). Survival analysis was performed after i.n. inoculation with 1×10e6 pfu of WSN-WT virus or 5×10e5 pfu of A/Hamburg/4/2009 v(H1N1) virus, which is a mouse adapted new swine origin pandemic H1N1 virus dose, respectively. All ten mice, which were immunized by WSN-A8 virus containing silent mutated NP, total protected of lethal challenge infection with the WSN virus (ca. 100×MLD50 dose) and the A/Hamburg/4/2009 v(H1N1) virus, (10×MLD dose), dose respectively.

FIG. 4b: Homolog immunity and cross-protection after 45 days post immunization with the attenuated WSN-A8 virus (Mice body weight loss curve). Weight change of BALB/c mice was controlled at time point after challenge i.n. inoculation with 1×10e6 pfu of WSN-WT virus or 5×10e5 pfu of A/Hamburg/4/2009 v(H1N1) virus, respectively. PBS infected mice were used as mock group mice. The average weight curve (in total %) with standard deviations of 5 mice in each group are depicted. All challenged mock mice, which were immunized with PBS instead the WSN-A8 virus are died within 7 days (n=10 mice).

FIG. 5a: Load of mice lung virus titer. Three mice were infected with 1×10e5 pfu of either WSN-WT or WSN-A8 virus. Three days p.i. all infected mice were euthanized and total lungs were collected. Virus titers were determined using lung homogenate (10% total lung homogenate in PBS) by plaque assay in MDCK cells.

FIG. 5b: Determination of total virus particles of different WSN viruses. By hemagglutination test (HA-test) was identified total virus particles of different NP mutant viruses (WSN-A2, WSN-A3, and WSN-A8) as well as WSN-WT virus, respectively. All investigated virus has a equal amount of infectious particles (3.5×10e6 PFU in 100 microliter). HA titers of the WSN-A2 virus containing 3 silent mutated NP gene, WSN-A3 virus containing 4 silent mutated NP gene, and WSN-WT virus are identical 1:64 in contrast the HA titer of the WSN-A8 mutant virus with 9 silent mutations is 1:256.

FIG. 5c: Packaging efficiency of silent mutations on the NP gene segment of IAV. After ultracentrifugation of WSN-WT, and WSN-A8 virus stocks mit equal PFU titre, vRNAs were isolated from pure virus pellet using the High Pure Viral RNA Kit (Roche) according to manufacturer's instruction. Synthesized cDNAs from 0.1 μg of total vRNA were used for the Real-Time PCR analysis. Using appropriate TaqMan probes of Universal ProbeLibrary Set (Roche) are analyzed the packaging effect of silent mutated NP gene by Real-Time-PCR analysis for both, the silent mutated segment 5 (NP) and not mutated segments 2 and 7 (PA, and M), respectively. Here is shown the vRNA incorporation level of three different segments from equal amount of infectious particles of the indicated viruses. Shown is one representative result of three independent experiments with similar data.

FIG. 5d: Cells were transfected with the mini genome RNP plasmids (pHW2000-WSN-PB2, -PB1, -PA, and —NP or nine silent mutated NP-A8) and the antisense Luciferase reporter gene construct flanked by a Pol I promotor and terminator sites. 24 h post transfection the relative polymerase activity from 3 separate samples was detected. As negative control cell lysates of only Luciferase reporter gene construct transfected cells was used.

FIG. 5e: Measurement of NP protein expression. The NP expression level was analysed by western blot using cells extracts of mini genome of the WSN-WT, and WSN-A8 mutant viruses transfected 293 cells. ERK2 protein as loading control and both the non-transfected 293 cell lysate and the lysate of negative control of luciferase assay were used as negative control.

FIG. 6A depicts illustrative amino acids positions 468-498 and the corresponding nucleotide sequence of the nucleoprotein of eight Influenza A strains (1: strain A/Mallard/Astrakhan/244/1982 H14N6, EMBL-Bank accession No M30764, nucleotide positions 1411-1542; 2: strain A/Brevig Mission/1/1918 H1N1, i.e. the 1918 pandemic influenza virus, EMBL-Bank accession No M30764: AY744935, nucleotide positions 1366-1497; 3: strain A/Puerto Rico/8/1934(Cambridge) H1N1, EMBL-Bank accession No J02147, nucleotide positions 1411-1542; 4: strain A/Hong Kong/1/1968 H3N2, EMBL-Bank accession No AF348180, nucleotide positions 1366-1497; 5: strain A/Berkeley/1/1968 H2N2, EMBL-Bank accession No CY033476, nucleotide positions 1391-1522; 6: strain A/Rotterdam/1957 H2N2, EMBL-Bank accession No CY077898, nucleotide positions 1406-1537; 7: strain A/Tokyo/3/1967 H2N2, EMBL-Bank accession No AY210096, nucleotide positions 1366-1497; 8: strain A/Terrassa/INS94/2009 H1N1, EMBL-Bank accession No CY083693, nucleotide positions 1375-1515).

FIG. 6B depicts illustrative amino acids positions 10-24 and the corresponding nucleotide sequence of the polymerase (PA) of four Influenza B strains (1: strain B/Lee/40, EMBL-Bank accession No AF102017, nucleotide positions 28-72; 2: strain B/Singapore/222/1979, EMBL-Bank accession No M16711, nucleotide positions 57-101; 3: strain B/Harbin/7/1994, EMBL-Bank accession No CY040446, nucleotide positions 28-72; 4: strain B/Yamagata/16/1988, EMBL-Bank accession No CY018770, nucleotide positions 42-86).

FIG. 6C depicts four examples of the last four amino acids positions, positions 723-726 and the corresponding nucleotide sequence of the polymerase (PA) of four Influenza B strains (1: strain B/Lee/40, EMBL-Bank accession No AF102017, nucleotide positions 2167-2181; 2: strain B/Singapore/222/1979, EMBL-Bank accession No M16711, nucleotide positions 2193-2207; 3: strain B/Harbin/7/1994, EMBL-Bank accession No CY040446, nucleotide positions 2167-2181; 4: strain B/Yamagata/16/1988, EMBL-Bank accession No CY018770, nucleotide positions 2181-2195).

FIG. 6D depicts three examples of amino acids positions 714-720 and the corresponding nucleotide sequence of the polymerase basic 1 protein (PB1) of four Influenza B strains (1: strain B/Lee/1940, EMBL-Bank accession No DQ792895, nucleotide positions 2154-2174; 2: strain B/Bangkok/143/1994, EMBL-Bank accession No CY019689, nucleotide positions 2141-2161; 3: strain B/Hong Kong/1351/02, EMBL-Bank accession No CY018867, nucleotide positions 2142-2162).

FIG. 6E depicts three examples of amino acids positions 17-25 and the corresponding nucleotide sequence of the polymerase basic 1 protein (PB1) of four Influenza B strains (1: strain B/Lee/1940, EMBL-Bank accession No DQ792895, nucleotide positions 63-89; 2: strain B/Bangkok/143/1994, EMBL-Bank accession No CY019689, nucleotide positions 50-76; 3: strain B/Hong Kong/1351/02, EMBL-Bank accession No CY018867, nucleotide positions 51-77).

FIG. 6F depicts six examples of amino acids positions 697-701 and the corresponding nucleotide sequence of the polymerase PA of four Influenza A strains (1: strain A/NYMC X-163 (NYMC X-157-St. Petersburg/8/2006) H1N1, EMBL-Bank accession No CY034129, nucleotide positions 2093-2107; 2: strain A/Brevig Mission/1/1918 H1N1, EMBL-Bank accession No DQ208311, nucleotide positions 2092-2106; 3: strain A/Singapore/1-MA12E/1957 H2N2, EMBL-Bank accession No CY087797, nucleotide positions 2104-2118; 4: strain A/Sydney/405A/2001 H3N2, EMBL-Bank accession No HQ325818, nucleotide positions 2092-2106; 5: strain A/mallard/Netherlands/65/2006 H5N3, EMBL-Bank accession No CY076942, nucleotide positions 2104-2118; 6: strain A/Vancouver/01/2009 H1N1, EMBL-Bank accession No CY073783, nucleotide positions 2090-2104).

FIG. 6G depicts illustrative amino acids positions 29-36 and the corresponding nucleotide sequence of the polymerase (PB2) of four Influenza B strains (1: strain B/Panama/45/90, EMBL-Bank accession No AF005737, nucleotide positions 108-131; 2: strain B/Taiwan/1838/2006, EMBL-Bank accession No CY040377, nucleotide positions 85-108; 3: strain B/Lisbon/02/1994, EMBL-Bank accession No CY022236, nucleotide positions 86-109; 4: strain B/Guangzhou/01/2007, EMBL-Bank accession No EU305612, nucleotide positions 108-131).

FIG. 6H depicts illustrative amino acids positions 758-763 and the corresponding nucleotide sequence of the polymerase (PB2) of four Influenza B strains (1: strain B/Panama/45/90, EMBL-Bank accession No AF005737, nucleotide positions 108-131; 2: strain B/Chile/3162/2002, EMBL-Bank accession No CY019586, nucleotide positions 2275-2292; 3: strain B/Houston/B69/2002, EMBL-Bank accession No CY018156, nucleotide positions 2275-2292; 4: strain B/Oklahoma/WRAIR1587P/2009, EMBL-Bank accession No CY069570, nucleotide positions 2275-2292).

### DETAILED DESCRIPTION OF THE INVENTION

It must be noted that as used herein, the singular forms “a”, “an”, and “the”, include plural references unless the context clearly indicates otherwise. Thus, for example, reference to “an antibody” includes one or more of such different antibodies and reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supersede any such material.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or” as used herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The present inventors had the idea to destabilize the 3′- or 5′ end of RNA segments by silent mutations so that the packaging mechanism might be disrupted. Surprisingly, they could show that silent mutations without any change of the amino acid sequence of the gene leads to attenuation of, for example, influenza A viruses. This attenuation (downshift of virus replication) can thus be used as a live attenuated vaccine against influenza viruses. Presently, there is only an effective and relatively safe live attenuated IAV vaccine available on the basis of temperature-sensitive mutations approved in the USA (FluMist-Influenza Vaccine) and, therefore, further live attenuated vaccines are neededpa. However, unlike FluMist, the technology of the present invention is independent of any particular “master” donor strain and can be applied readily to any emerging influenza virus as a whole. This is of particular significance for dealing with seasonal epidemics and with pandemics. In fact, the technology of the present invention allows the fast generation of a “tailored to need” influenza vaccine, since synonymous mutations can easily be introduced at any gene segment within its RNA packaging signals and “complete” influenza viruses can be generated by use of a reverse genetics system such as one described in WO 00/60050 or in accordance with the methods described in the appended Examples.

As a model gene, the present inventors used the nucleoprotein (NP) of influenza A viruses. NP is associated with many functions during viral replication including host range restriction. (Scholtissek (1995), Virus Genes 11:209-215; Portela and Digard (2002), J Gen Virol 83:723-734. When about 600 sequences of the NP gene from Gen Bank were compared at the 3″ends of the cRNA a highly conserved region of about 30 nucleotides within the open reading frame was found, in which even silent mutations were not allowed. This suggests that integrity of the RNA structure in this region is crucial for influenza A virus replication. To analyze the impact of these conserved nucleotides mutant viruses with one or more silent mutations in the respective region of the NP gene of two different influenza A virus strains (WSN, FPV) were generated. There were significant differences in the growth of wild type and viruses with up to nine silent mutations indicating a growth disadvantage of viruses carrying silent mutations at the 3″end of the NP cRNA. Since many silent mutations are necessary for attenuation a reversion to wild type virus is extremely improbable.

In the present approach the inventors tested the attenuation of influenza A viruses by introduction of silent mutations into the NP gene by infecting mice with mutant and wild type WSN as a model for the creation of a live attenuated vaccine. The vaccinated mice with the mutant WSN virus and a reassortant PR8 virus which contains the silent mutated NP gene from WSN virus survived from the lethal challenge dose of wild type WSN virus and the PR8 virus carrying the wild type NP gene of A/WSN/33, respectively. The results for the nucleoprotein (NP) of influenza A viruses provide convincing evidence that this is a practicable strategy. This principle can be reasonably extrapolated to other genes of influenza viruses, in particular influenza virus A as well as to influenza virus B or C which a segmented genome, too.

Accordingly, the present inventors developed a systematic approach how to attenuate viruses having a segmented genome, in particular influenza viruses with the aim of generating live, attenuated viruses having a segmented genome, in particular influenza viruses. Thus, the present invention provides a method for obtaining a live, attenuated virus having a segmented genome, in particular a live, attenuated influenza virus, said method comprising

(a) comparing a plurality of nucleotide sequences of RNA packaging signals of a gene segment of a virus having a segmented genome (preferably an influenza virus);
(b) identifying (a) conserved nucleotide(s) at the third position of a codon within a RNA packaging signal;
(c) substituting said conserved nucleotide(s) by (a) synonymous nucleotide(s) (i.e., introducing a synonymous mutation);
(d) producing a virus having a segmented genome (preferably an influenza virus) comprising said synonymous nucleotide(s);
(e) determining whether a virus having a segmented genome (preferably an influenza virus) containing said synonymous nucleotide(s) at the position(s) corresponding to the respective position(s) within the RNA packaging signal of a virus having a segmented genome (preferably an influenza virus) not containing said synonymous nucleotide(s) is attenuated in comparison to the same virus having a segmented genome (preferably an influenza virus) not containing said synonymous nucleotide(s) within the respective RNA packaging signal; and
(f) obtaining said live, attenuated virus having a segmented genome (preferably an influenza virus).

Preferably, the virus having a segmented genome is a virus of the family orthomyxoviridae, bunyaviridae or arenaviridae. More preferably, the virus having a segmented genome is an influenza A virus, influenza B virus or influenza C virus, with influenza A virus being preferred.

In a preferred embodiment of the above method, the nucleotide sequences of RNA packaging signals of a gene segment is from influenza A virus.

In another preferred embodiment of the above method, the gene segment is from the influenza virus NP, PA, PB1, PB2, HA, NA, M, NS, BM2, or NS-2 gene.

In still another preferred embodiment of the above method, the RNA packaging signal comprises 9-250 nucleotides of the 5′ end and/or 9-250 nucleotides of the 3′ end of a gene segment of an influenza virus.

In the above method, it is preferred that the plurality of nucleotide sequences of RNA packaging signals of a gene segment of an influenza virus comprises at least 2, 5, or 10, more preferably at least 20, particularly preferable at least 50, 100, 150, 200, 250, 300, 350, 400, 450, 500, 550, 600 of said nucleotide sequences. Accordingly, it is envisaged that preferably at least 2, 5 or 10, more preferably at least 20, particularly preferable at least 50, 100, 150, 200, 250, 300, 350, 400, 450, 500, 550 or 600 nucleotide sequences of RNA packaging signals of a gene segment of an influenza virus are compared.

In the context of the above method, it is preferred that a nucleotide (at the third position of a codon within a RNA packaging signal) is conserved, if it is present in at least 60% of the nucleotide sequences that are compared.

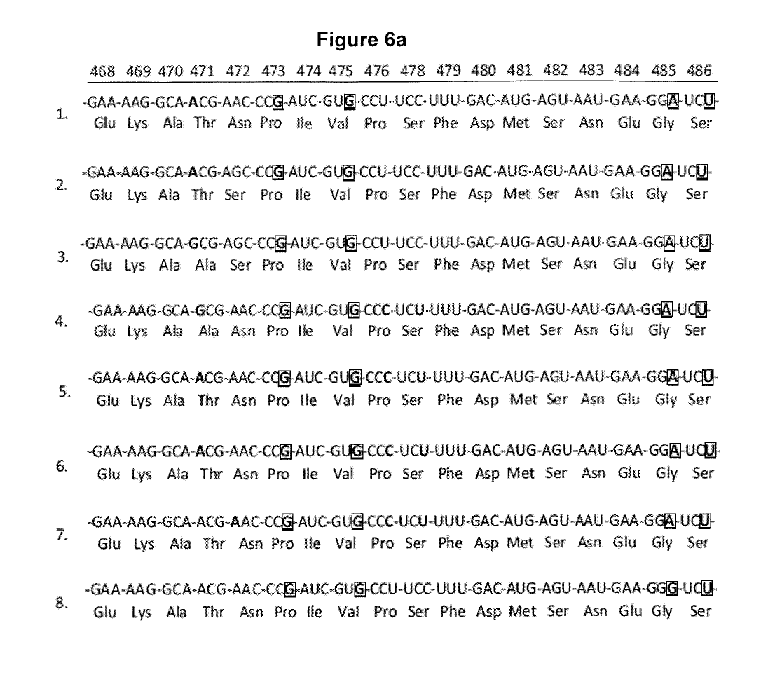

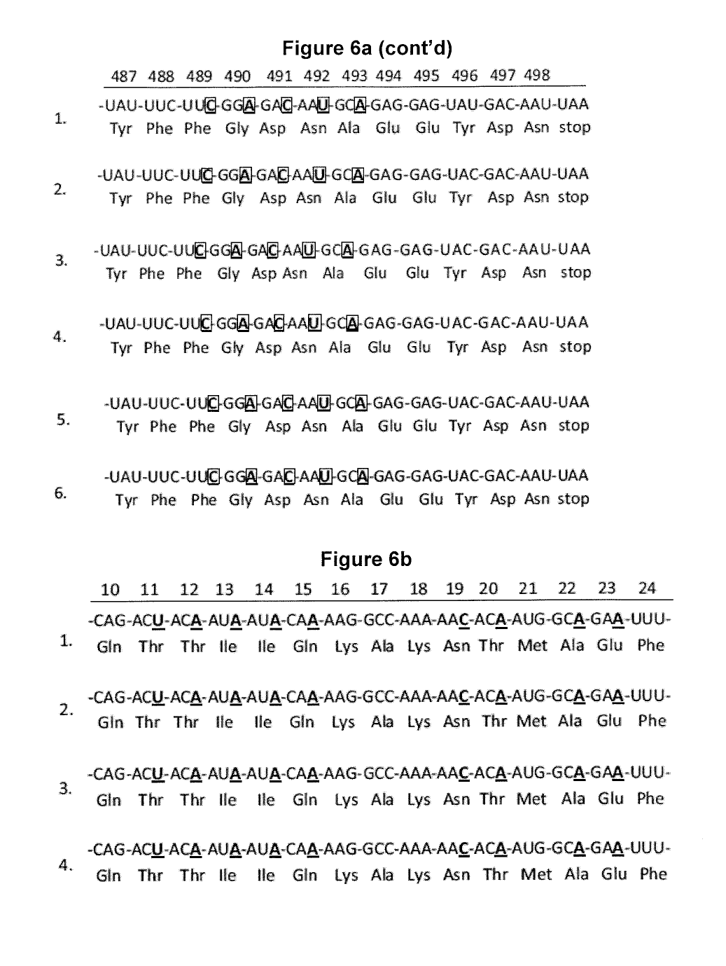

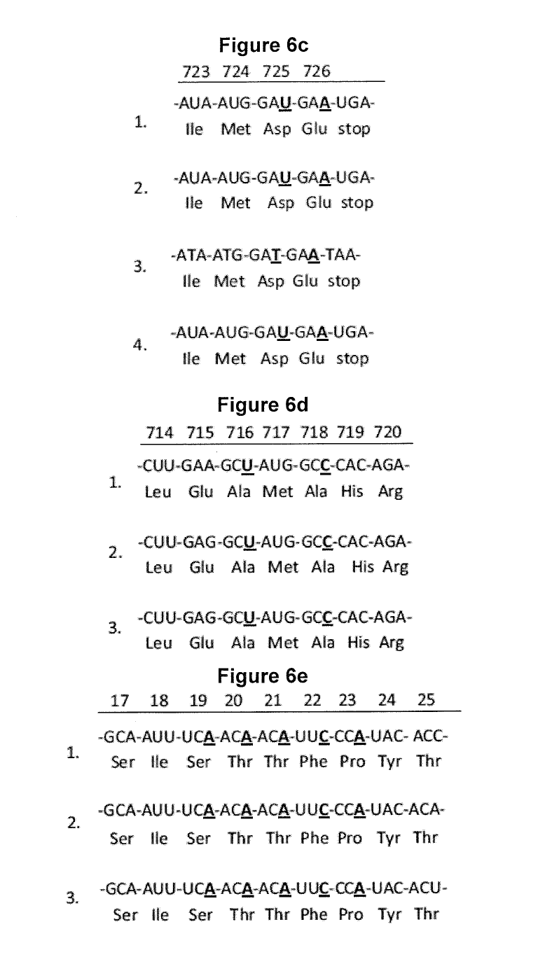

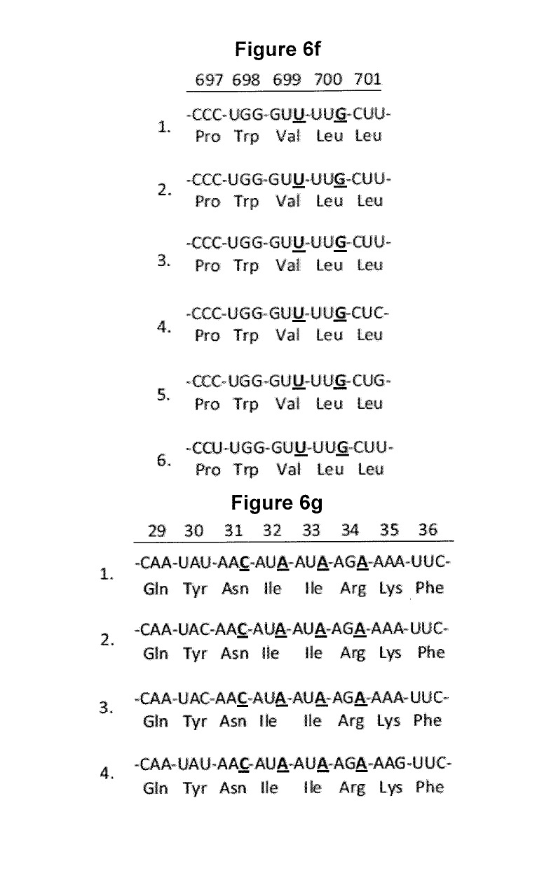

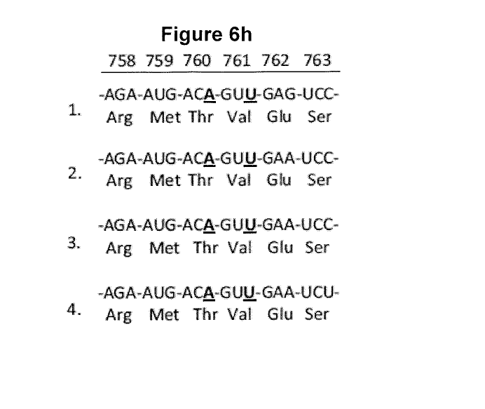

Specifically, in order to compare a plurality of nucleotide sequences (or amino acid sequences) of, for example, RNA packaging signals, a skilled artisan can use means and methods well-known in the art, e.g., alignments, either manually or by using computer programs such as BLAST2.0, which stands for Basic Local Alignment Search Tool, MAFFT, or ClustalW or any other suitable program which is suitable to generate sequence alignments. A preferred sequence alignment applied in the context of the above method of the present invention is a multiple sequence alignment; see FIG. 6 which illustrates the comparison of nucleotide sequences of RNA packaging signals.

A multiple sequence alignment is an extension of pairwise alignment to incorporate more than two sequences at a time. Multiple alignment methods align all of the sequences in a given query set. For the purpose of the present invention a multiple alignment is preferably used in identifying conserved sequence regions across a group of RNA packaging sequences from different influenza viruses such as those described herein. A preferred multiple sequence alignment program (and its algorithm) is ClustalW, Clusal2W or ClustalW XXL (see Thompson et al. (1994) Nucleic Acids Res 22:4673-4680). Note that Clustal2W and ClustalW XXL are further developments of ClustalW. The skilled artisan is readily in a position to retrieve influenza virus gene segment sequences such as those comprising the NP, HA, NS, NS-2, NA, PA, PB1; PB2, M or BM2 from known data bases such as Gen Bank. Following that, the skilled artisan is well aware of the coding sequence of an influenza virus gene segment. On the basis of the coding sequence, the skilled artisan can determine codons as well as 5′ and 3′ untranslated regions. Accordingly, the skilled person instructed by the present invention that RNA packaging signals comprise preferably all nucleotides of the 5′ non-coding region and about 9-250 (including 9, 10, 11, 12, 13, 14, 15, 20, 25, 30, 35, 40, 45, 50, 60, 70, 80, 90, 100, 110, 120, 130, 140, 150, 160, 170, 180, 190, 200, 210, 220, 230, 240) nucleotides adjacent (5′→3′) to said nucleotides of the 5′ non-coding region of an influenza gene (i.e., vRNA or cRNA) and/or it comprises all nucleotides of the 3′ non-coding region and about 20-230 (including 20, 21, 22, 23, 24, 25, 30, 35, 40, 45, 50, 60, 70, 80, 90, 100, 110, 120, 130, 140, 150, 160, 170, 180, 190, 200, 210, 220) nucleotides adjacent (3′→5′) to said nucleotides of the 3′ non-coding region, of an influenza gene (i.e., vRNA or cRNA), respectively. Accordingly, the “comparing step” comprises aligning nucleotide sequences;; see FIG. 6 which illustrates the comparison of nucleotide sequences of RNA packaging signals by way of a multiple sequence alignment.

Once nucleotide sequences of RNA packaging signals are compared (aligned) as described herein, the skilled artisan can readily identify (a) conserved nucleotide(s) at the third position of a codon within a RNA packaging signal;; see FIG. 6 which illustrates the comparison of nucleotide sequences of RNA packaging signals and the identification of conserved nucleotides as described herein.

Following the identification of (a) conserved nucleotide(s) at the third position of a codon within a RNA packaging signal, said nucleotide(s) is/are substituted by (a) synonymous nucleotide(s), i.e., a synonymous mutation is introduced.

Following the introduction of one or more synonymous mutations at the third position of a codon within a RNA packaging signal, an influenza virus comprising said synonymous nucleotide(s) is produced as described herein or as is commonly known in the art, for example, by a reverse genetic system as described in Neumann et al. (2000), Proc. Natl. Acad. Sci 97:6108-6113.

Following the production of an influenza virus comprising said synonymous nucleotide(s) it is determined whether an influenza virus containing said synonymous nucleotide(s) at the position(s) corresponding to the respective position(s) within the RNA packaging signal of an influenza virus not containing said synonymous nucleotide(s) is attenuated in comparison to the same influenza virus not containing said synonymous nucleotide(s) within the respective RNA packaging signal.

Furthermore, the present invention provides a composition comprising a live, attenuated influenza viruses, in particular influenza virus A, said virus having a silent mutation at one or more positions located in the 5′- and/or 3′ region of viral genes, which serve as packaging signals. These positions are further described in detail herein. In contrast to the present invention, WO 2011/044561 provides a “landscape” approach in that influenza viruses are attenuated by introducing nucleotide substitutions which result in the rearrangement of preexisting codons of one or more protein encoding sequences and changes in codon pair bias. However, unlike the present invention, WO 2011/044561 does not provide specific positions in gene segments of influenza viruses, in particular in IAV, that should be substituted by introducing a synonymous mutation at the third base of a codon.

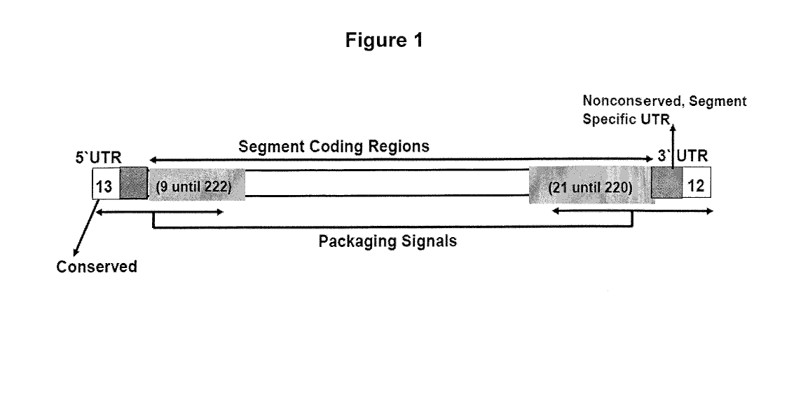

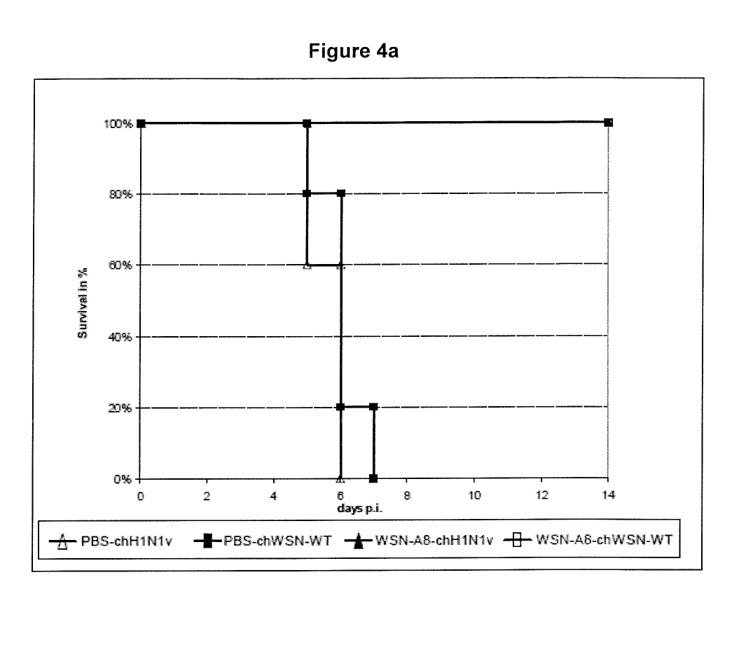

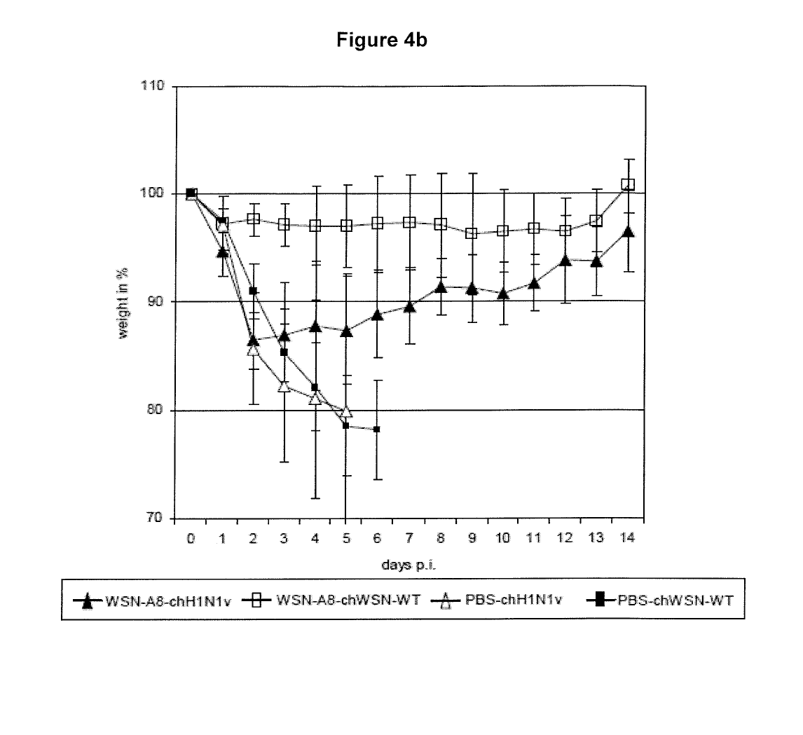

A “packaging signal” when used herein constitutes of a stretch of nucleotides that are required by viruses with segmented genomes, such as influenza viruses, in particular influenza virus A, influenza virus B or influenza virus C, to package its gene segments; for illustration see FIG. 1 of the present application, FIG. 4 of Hutchinson et al. (2010) J Gen Virol 91:313-328 or Fields “Virology” 5^{th} Edition, Lippincott Williams & Wilkins, Chapter 47, page 1669, FIG. 47.23). For RNA packaging signals; see also Liang et al. (2005), J Virol 79:10348-10355; Wit et al. (2006), Vaccine 24:6647-6450, Gog et al. (2007), Nucleic Acids Res 35:1897-1907, Marsh et al. (2007), J Virol 81:9727-9236, Ozawa et al. (2007), J Virol 81:30-41, Hutchinson et al. (2009), J Gen Virol 91(Pt 2):313-328, Fujii et al. (2003), Proc Natl Acad Sci USA 100:2002-2007, Hutchinson et al. (2008), J Virol 82:11869-11879, Fujii et al. (2005), J Virol 79:3766-3674 (all incorporated by reference). Because of the packaging signal the gene segments become packaged and later enveloped to reconstitute a viral particle. Influenza virus packaging signals are located within an open reading frame at the 5′- and/or 3′-end. Preferably, a “packaging signal” comprises all nucleotides, preferably the completely conserved region encompassing 13 nucleotides of the 5′ non-coding region and about 9-250 (including 9, 10, 11, 12, 13, 14, 15, 20, 25, 30, 35, 40, 45, 50, 60, 70, 80, 90, 100, 110, 120, 130, 140, 150, 160, 170, 180, 190, 200, 210, 220, 230, 240) nucleotides adjacent (5′→3′) to said nucleotides of the 5′ non-coding region of an influenza gene segment (i.e., vRNA or cRNA) and/or it comprises all nucleotides, preferably the completely conserved region encompassing 12 nucleotides of the 3′ non-coding region and about 20-230 (including 20, 21, 22, 23, 24, 25, 30, 35, 40, 45, 50, 60, 70, 80, 90, 100, 110, 120, 130, 140, 150, 160, 170, 180, 190, 200, 210, 220) nucleotides adjacent (3′→5′) to said nucleotides of the 3′ non-coding region, of an influenza gene segment (i.e., vRNA or cRNA), respectively.

More preferably, the PB2 gene (i.e., vRNA or cRNA) comprises all nucleotides of the 5′ non-coding region and about 10-160 (including 20, 30, 40, 50, 60, 70, 80, 90, 100, 110, 120, 130, 140, 150) nucleotides adjacent (5′→3′) to said nucleotides of the 5′ non-coding region and/or it comprises all nucleotides of the 3′ non-coding region and about 20-160 (including 20, 30, 40, 50, 60, 70, 80, 90, 100, 110, 120, 130, 140, 150) nucleotides adjacent (3′→5′) to said nucleotides of the 3′ non-coding region, respectively.

More preferably, the PB1 gene (i.e., vRNA or cRNA) comprises all nucleotides of the 5′ non-coding region and about 10-100 (including 20, 30, 40, 50, 60, 70, 80, 90) nucleotides adjacent (5′→3′) to said nucleotides of the 5′ non-coding region and/or it comprises all nucleotides of the 3′ non-coding region and about 10-100 (including 20, 30, 40, 50, 60, 70, 80, 90) nucleotides adjacent (3′→5′) to said nucleotides of the 3′ non-coding region, respectively.

More preferably, the PA gene (i.e., vRNA or cRNA) comprises all nucleotides of the 5′ non-coding region and about 10-50 (including 11, 12, 13, 14, 15, 20, 25, 30, 40, 45) nucleotides adjacent (5′→3′) to said nucleotides of the 5′ non-coding region and/or it comprises all nucleotides of the 3′ non-coding region and about 10-50 (including 20, 21, 22, 23, 24, 25, 30, 40, 45) nucleotides adjacent (3′→5′) to said nucleotides of the 3′ non-coding region, respectively.

More preferably, the HA gene (i.e., vRNA or cRNA) comprises all nucleotides of the 5′ non-coding region and about 9-50 (including 10, 11, 12, 13, 14, 15, 20, 25, 30, 40, 45) nucleotides adjacent (5′→3′) to said nucleotides of the 5′ non-coding region and/or it comprises all nucleotides of the 3′ non-coding region and about 60-120 (including 70, 80, 90, 100, 110) nucleotides adjacent (3′→5′) to said nucleotides of the 3′ non-coding region, respectively.

More preferably, the NP gene (i.e., vRNA or cRNA) comprises all nucleotides of the 5′ non-coding region and about 40-100 (including 50, 60, 70, 80, 90) nucleotides adjacent (5′→3′) to said nucleotides of the 5′ non-coding region and/or it comprises all nucleotides of the 3′ non-coding region and about 100-150 nucleotides (including 110, 120, 130, 140) adjacent (3′→5′) to said nucleotides of the 3′ non-coding region, respectively.

More preferably, the NA gene (i.e., vRNA or cRNA) comprises all nucleotides of the 5′ non-coding region and about 100-220 (including 110, 120, 130, 140, 150, 160, 170, 180, 183, 190, 200, 210) nucleotides adjacent (5′→3′) to said nucleotides of the 5′ non-coding region and/or it comprises all nucleotides of the 3′ non-coding region and about 100-220 (including 110, 120, 130, 140, 150, 157, 160, 170, 180, 190, 200, 210) nucleotides adjacent (3′→5′) to said nucleotides of the 3′ non-coding region, respectively.

More preferably, the M gene (i.e., vRNA or cRNA) comprises all nucleotides of the 5′ non-coding region and about 100-250 (including 110, 120, 130, 140, 150, 160, 170, 180, 190, 200, 210, 220, 222, 230, 240) nucleotides adjacent (5′→3′) to said nucleotides of the 5′ non-coding region and/or it comprises all nucleotides of the 3′ non-coding region and about 100-230 (including 110, 120, 130, 140, 150, 160, 170, 180, 190, 200, 210, 220, 230, 240) nucleotides adjacent (3′→5′) to said nucleotides of the 3′ non-coding region, respectively.

More preferably, the NS gene (i.e., vRNA or cRNA) comprises all nucleotides of the 5′ non-coding region and about 10-80 (including 20, 30, 35, 40, 50, 60, 70) nucleotides adjacent (5′→3′) to said nucleotides of the 5′ non-coding region and/or it comprises all nucleotides of the 3′ non-coding region and about 10-80 (including 20, 30, 35, 40, 50, 60, 70) nucleotides adjacent (3′→5′) to said nucleotides of the 3′ non-coding region, respectively.

As indicated above, in the context of the invention, an “influenza virus” refers to the causative agent of flu. The term refers to the lipid-enveloped particle that contains its genome, which has seven or eight strands of negative-sense RNA. When used herein the term “influenza virus” encompasses influenza virus A (IAV), influenza virus B (IBC) and influenza virus C (ICV).

As used herein, a “live non-attenuated influenza virus” refers to a living enveloped RNA virus with a segmented genome consisting of seven or eight single-stranded negative RNA segments, and belonging to the family of Orthomyxoviridae.

A “live attenuated influenza virus” as used herein, refers to a living influenza virus strain that displays at least an attenuated virulence, but is still capable of eliciting an immune response. “Attenuated virulence” means that an attenuated influenza virus is in comparison to a wild-type influenza virus diminished in plaque size, growth and/or lethality in test animals such as a monkey, pig, horse, cat, dog, mouse, or a fowl, e.g., domestic fowl or domestic duck. “Diminished” includes 10% reduction, preferably 20% or 30% %, more preferably 40% or 50%, even more preferably 60% or 70%, particularly preferred 80 or 85% and most particularly preferred 90% % reduction of the attenuated virus as regards growth and/or lethality in comparison to the wild-type virus. Standard plaque assays, growth assays and methods for testing lethality in a test animal are well known in the art.

The term “influenza” when used herein, apart from being part of the name of the influenza viruses of the present invention refers to the disease and/or symptoms caused by influenza viruses. Symptoms of influenza can start quite suddenly one to two days after infection. Usually the first symptoms are chills or a chilly sensation, but fever is also common early in the infection, with body temperatures ranging from 38-39° C. up to 42° C. Many subjects are so ill that they are confined to bed for several days, with aches and pains throughout their bodies, which are worse in their backs and legs. Symptoms of influenza may include fever and extreme coldness (chills shivering, shaking (rigor)), cough, nasal congestion, body aches, especially joints and throat, fatigue, headache, irritated, watering eyes, reddened eyes, skin (especially face), mouth, throat and nose, in children, gastrointestinal symptoms such as diarrhea and abdominal pain (may be severe in children with influenza B).

An “immune response” to an antigen or vaccine composition is the development in a subject of a humoral and/or a cell-mediated immune response to molecules present in the antigen or vaccine composition of interest. For purposes of the present invention, a “humoral immune response” is an antibody-mediated immune response and involves the generation of antibodies with affinity for the antigen/vaccine of the invention, while a “cell-mediated immune response” is one mediated by T-lymphocytes and/or other white blood cells. A “cell-mediated immune response” is elicited by the presentation of antigenic epitopes in association with Class I or Class II molecules of the major histocompatibility complex (MHC). This activates antigen-specific CD4+ T helper cells or CD8+ cytotoxic T lymphocyte cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lympho-proliferation (lymphocyte activation) assays, CTL cytotoxic cell assays, by assaying for T-lymphocytes specific for the antigen in a sensitized subject, or by measurement of cytokine production by T cells in response to restimulation with antigen. Such assays are well known in the art. See, e.g., Erickson et al., J. Immunol. (1993) 151:4189-4199; Doe et al., Eur. J. Immunol. (1994) 24:2369-2376. An “immunologically effective amount” or an “effective immunizing amount”, used interchangeably herein, refers to the amount of antigen or vaccine sufficient to elicit an immune response, either a cellular (T cell) or humoral (B cell or antibody) response, as measured by standard assays known to one skilled in the art. The effectiveness of an antigen as an immunogen, can be measured either by proliferation assays, by cytolytic assays, such as chromium release assays to measure the ability of a T cell to lyse its specific target cell, or by measuring the levels of B cell activity by measuring the levels of circulating antibodies specific for the antigen in serum.

The “immune response” is preferably a “protective” immune response. A “protective” immune response refers to the ability of a vaccine to elicit an immune response, either humoral or cell mediated or both, which serves to protect the mammal from influenza.

The protection provided need not be absolute, i.e., influenza need not be totally prevented or influenza viruses be totally eradicated, if there is a statistically significant improvement compared with a control population of mammals. Protection may be limited to mitigating the severity or rapidity of onset of symptoms of influenza. The immune response is preferably sufficient to treat and/or prevent from influenza. When used herein, the term “treating” or “preventing” influenza means to at least inhibit virus replication of IAV, IBA or ICV, respectively, to inhibit influenza transmission, and/or to prevent IAV, IBV or ICV, respectively, from establishing itself in a subject, and/or to ameliorate or alleviate the symptoms of the disease caused by IAV, IBV or ICV, respectively. The term “vaccinating” or “immunizing” (can be used interchangeably) when used herein includes treatment and/or prevention of influenza.

Preferably, a protective immune response includes the following: an influenza virus of the present invention (in particular IAV) or a composition comprising such IAV as described herein confers to a serum sample from a subject (including a mammal or bird), to which subject there has been administered at least one dose of about 10⁴ to about 10⁵ PFU/kg of the influenza virus of the present invention, a hemagglutinin inhibition (HI) titer of preferably at least about 1:520, when tested against the same influenza virus (particularly IAV) not having one or more of the silent mutations as described herein.

Also or alternatively, a protective immune response preferably includes that an influenza virus of the present invention (in particular IAV) or a composition comprising such IAV as described herein confers protection against a 10-100-fold lethal dosis against wild-type IAV, i.e., not having one or more of the silent mutations as described herein.

In some embodiments an attenuated influenza virus may be a strain of influenza that is cold-adapted. In some embodiments such an attenuated strain is temperature-sensitive. In contrast thereto, a “killed influenza virus” or “inactivated influenza virus” refers to inactivated influenza viruses obtained by known methods, the most common of which is to grow the virus in fertilized hen eggs, to purify it and to inactivate it, for example, by treatment with detergent. In this regard the words “killed” vs. “live” or “living” are used for easy of reference and are not intended to imply that viruses should be considered living entities. These words merely serve in distinguishing viruses that do not and viruses that do have the ability of the virus to infect a host cell and pass on genetic information to future generations.

The present invention relates to an attenuated influenza A virus (IAV) that is suitable for use in a vaccine. Provided are also compositions with a live, attenuated influenza A virus (IAV), a use thereof, as well as an IAV PA gene. Provided is further a method for preparing a live, attenuated IAV.

The terms “vaccine” or “vaccine composition” are used interchangeably herein and refer to a composition comprising at least one immunologically active component that induces an immune response in a subject against influenza viruses, and/or protects the subject from influenza or possible death due to influenza, and may or may not include one or more additional components that enhance the immunological activity of the active component. A vaccine may additionally comprise further components typical to pharmaceutical compositions. Said at least one immunologically active component is one or more of the influenza viruses of the present invention. The vaccine of the present invention is for human and/or veterinary use.

A live, attenuated IAV may be prepared by (a) introducing into into a host cell (i) a vector comprising the IAV PA gene as described herein and (ii) a plurality of vectors comprising the remaining IAV genes required to form an infectious IAV; and (b) isolating infectious IAV from said host cell. The remaining IAV genes required to form an infectious IAV are a PB1 gene, PB2 gene, HA gene, NA gene, NS1 gene, NS2 gene, M1 gene, M2 gene and a NP gene.

Alternatively, a live, attenuated IAV can be prepared by (a) culturing the host cell comprising a vector comprising the IAV PA gene as described herein and a plurality of vectors comprising the remaining IAV genes required to form an infectious IAV; and (b) isolating infectious IAV from said host cell. Said remaining IAV genes are a PB1 gene, PB2 gene, HA gene, NA gene, NS1 gene, NS2 gene, M1 gene, M2 gene and a NP gene.

The methods for the preparation of a live, attenuated IAV further comprise preferably the step of (c) formulating said IAV with a pharmaceutically acceptable carrier.

The present invention also envisages a live, attenuated IAV obtainable by the afore-described methods.

As indicated above, there are only two similarly effective and relatively safe live attenuated IAV vaccines available on the basis of temperature-sensitive mutations approved in the USA (FluMist-Influenza Vaccine, Aviron, Chen et al., 2008). In 1996 Herlocher et al. published the results of their analysis on all the virulent or attenuated cold adapted influenza A lines available at the University of Michigan (Virus Research (1996) 42, 11-25). In U.S. Pat. No. 7,344,722 B1 the same group disclosed identified differences between the sequences of cold-adapted and wild type strains A/Ann Arbor/6/60 H2N2 as well as a resulting change in secondary structure. They concluded that attenuated strains and virulent strains differed in point mutations in the six internal genes. As decisive in cold-adaptation they suggested the PG gene, namely silent nucleotides 141 and 1933 therein. Four further differences, three in the NP gene and one in the PA gene of A/Ann Arbor/6/60 H2N2 were attributed to host adaption.

As mentioned above, an intact virion or infectious IAV is only formed if all 8 gene segments are effectively incorporated into viral particles. The late step, packaging, of viral replication is obviously most crucial. Presumably RNA-RNA interactions are involved (Fujii, Y., et al., PNAS (2003) 100, 4, 2002-2007; Hutchinson, E. C., et al., Vaccine (2009) 27, 6270-6275). However, the exact incorporation mechanism of IAV RNA segments is so far not completely elucidated. There exist two different theories, known as the random and the selective incorporation models. The random incorporation model is supported by the observation that infectious virions do sometimes possess more than eight vRNP segments (Enami et al., 1991; Bancroft and Parslow, 2002; Gao et al., 2010). The second model, the selective incorporation model, suggests that each vRNA segment acts individually with another one, allowing each segment to be packaged selectively (Fujii, 2003, supra; Liang, Y., et al., Journal of Virology (2005) 79, 16, 10348-10355; Gao and Palese 2009; Hutchinson, 2009, supra).

Ozawa et al., (2007) have shown already by deletion of fragments at the 3′- or 5′ end of each RNA segment that these fragments extending into the coding region are necessary for selective packaging (Fujii et al., 2003; Fujii et al., 2005; Marsh et al., 2007).

This aspect of the present invention is based on the surprising finding that it is possible to destabilize this area by silent mutations so that the packaging mechanism is disrupted. As a result a virus with such a silent mutation is several magnitudes slower in replication, thereby allowing the host organism more than sufficient time to develop an immune response. Therefore, preferably such a virus does essentially not cause disease, more preferably it does not cause disease. In particular, positions of the coding region of the nucleic acid sequence of segment 5 of the influenza A were identified that can be used to control attenuation of the virus without affecting the encoded amino acid sequence. Accordingly, silent mutations without any change to the amino acid sequence of the gene lead to attenuation of influenza A viruses. Based on the findings of the present inventors it is also envisaged that silent mutations without any change to the amino acid sequence of the gene lead to attenuation of influenza B viruses.

In one aspect of the present invention this attenuation, for example, inhibition of virus replication, is used as a live attenuated vaccine against influenza viruses. The positions according to a first aspect of the invention, identified by the inventors, were unexpected, since Hutchinson et al. had previously analysed segment 5 of the influenza A virus by mutational analysis for positions sensitive in terms of mutational disruption (Vaccine, (2009) 27, 6270-6275). Hutchinson et al. identified in the corresponding region of segment 5 only amino acid positions 464 and 466 of the nucleoprotein according to SEQ ID NO: 1 as being sensitive in terms of packing defects, corresponding to base triplets 1395-1397 and 1441-1443 of the nucleic acid sequence of SEQ ID NO: 1. These positions are located between 100 and 108 nucleotides before the end of the stop codon defining the 3′-end of the sequence encoding the nucleoprotein of Influenza A virus. In other words, this region stretches from nucleotide positions 100 to 108, when counted from the 3′-end. Positions identified by the inventors as suitable for rendering an influenza A virus attenuated are located 73 to 19 nucleotides before the end of the stop codon at the 3′-end of the sequence encoding the nucleoprotein of Influenza A virus.

The term “nucleic acid molecule” as used herein refers to any nucleic acid in any possible configuration, such as single stranded, double stranded or a combination thereof. Nucleic acids include for instance DNA molecules, RNA molecules, analogues of the DNA or RNA generated using nucleotide analogues or using nucleic acid chemistry, locked nucleic acid molecules (LNA), protein nucleic acids molecules (PNA) and tecto-RNA molecules (e.g. Liu, B., et al., J. Am. Chem. Soc. (2004) 126, 4076-4077). A PNA molecule is a nucleic acid molecule in which the backbone is a pseudopeptide rather than a sugar. Accordingly, PNA generally has a charge neutral backbone, in contrast to for example DNA or RNA. Nevertheless, PNA is capable of hybridising at least complementary and substantially complementary nucleic acid strands, just as e.g. DNA or RNA (to which PNA is considered a structural mimic). An LNA molecule has a modified RNA backbone with a methylene bridge between C4′ and O2′, which locks the furanose ring in a N-type configuration, providing the respective molecule with a higher duplex stability and nuclease resistance. Unlike a PNA molecule an LNA molecule has a charged backbone. DNA or RNA may be of genomic or synthetic origin and may be single or double stranded. Such nucleic acid can be e.g. mRNA, cRNA, vRNA, synthetic RNA, genomic DNA, cDNA synthetic DNA, a copolymer of DNA and RNA, oligonucleotides, mixed polymers, both sense and antisense strands, or may contain non-natural or derivatized nucleotide bases. A respective nucleic acid may furthermore contain non-natural nucleotide analogues and/or be linked to an affinity tag or a label. When referred to herein the terms “nucleotide sequence(s)”, “polynucleotide(s)”, “nucleic acid sequence(s)” “nucleic acid(s)”, “nucleic acid molecule” are used interchangeably.

Many nucleotide analogues are known and can be employed in the methods of the invention. A nucleotide analogue is a nucleotide containing a modification at for instance the base, sugar, or phosphate moieties. As an illustrative example, a substitution of 2′-OH residues of siRNA with 2′F, 2′O-Me or 2′H residues is known to improve the in vivo stability of the respective RNA. Modifications at the base moiety include natural and synthetic modifications of A, C, G, and T/U, different purine or pyrimidine bases, such as uracil-5-yl, hypoxanthin-9-yl, and 2-aminoadenin-9-yl, as well as non-purine or non-pyrimidine nucleotide bases. Other nucleotide analogues serve as universal bases. Universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

The term “position” when used in accordance with the disclosure means the position of either an amino acid within an amino acid sequence depicted herein or the position of a nucleotide within a nucleic acid sequence depicted herein. The term “corresponding” as used herein also includes that a position is not only determined by the number of the preceding nucleotides/amino acids, but is rather to be viewed in the context of the circumjacent portion of the sequence. Accordingly, the position of a given nucleotide in accordance with the disclosure which may be substituted may very due to deletion or addition of nucleotides elsewhere in a (mutant or wild-type) Influenza virus nucleotide sequence, including the promoter and/or any other regulatory sequences or gene (including exons and introns).

In this regard it is also noted that data base entries of a nucleotide sequence of an Influenza virus may vary in their coverage of non-translated regions, thereby identifying different nucleic acid positions, even though the length of the coding region is unchanged/the same. Similarly, the position of a given amino acid in accordance with the present disclosure which may be substituted may vary due to deletions or additional amino acids elsewhere in an Influenza virus protein.

Thus, when a position is referred to as a “corresponding position” in accordance with the disclosure it is understood that nucleotides/amino acids may differ in terms of the specified numeral but may still have similar neighbouring nucleotides/amino acids. Such nucleotides/amino acids which may be exchanged, deleted or added are also included in the term “corresponding position”.

Specifically, in order to determine whether a nucleotide residue of a nucleotide sequence of an influenza virus gene, that is different from a nucleotide residue of a nucleotide sequence of a known influenza virus nucleotide sequence (in particular, a gene), corresponds to a certain position in the nucleotide sequence of said known nucleotide sequence e, a skilled artisan can use means and methods well-known in the art, e.g., alignments, either manually or by using computer programs such as BLAST 2.0, which stands for Basic Local Alignment Search Tool or ClustalW or any other suitable program which is suitable to generate sequence alignments. Accordingly, the nucleotide sequence (for example, that of an influenza virus gene) of a known wild-type Influenza virus strain may serve as “subject sequence” or “reference sequence”, while the nucleotide sequence of a gene of interest of a virus different from the wild-type virus strain described herein serves as “query sequence”. The terms “reference sequence”, “subject sequence” and “wild type sequence” are used interchangeably herein. Any of the Influenza virus nucleotide sequences disclosed herein can serve as a reference sequence.

Similarly, in order to determine whether an amino acid residue of the amino acid sequence of an influenza virus polypeptide, that is different from an amino acid residue of a polypeptide of a known polypeptide, corresponds to a certain position in the amino acid sequence of said known polypeptide, a skilled artisan can use means and methods well-known in the art, e.g., alignments, either manually or by using computer programs such as BLAST2.0, or ClustalW or any other suitable program which is suitable to generate sequence alignments. Accordingly, the amino acid sequence a polypeptide of a known wild-type virus strain may serve as “subject sequence” or “reference sequence”, while the amino acid sequence of a protein of interest of a virus different from the wild-type virus strain described herein serves as “query sequence”. The terms “reference sequence”, “subject sequence” and “wild type sequence” are used interchangeably herein. Any of the Influenza virus amino acid sequences disclosed herein can serve as a reference sequence.

Also, in order to determine whether a nucleotide residue or amino acid residue in a given Influenza virus nucleotide/amino acid sequence corresponds to a certain position in any one of the nucleotide sequences disclosed herein or the amino acid sequence disclosed herein, respectively, the skilled person can use means and methods well-known in the art, e.g., alignments, either manually or by using computer programs such as BLAST2.0, which stands for Basic Local Alignment Search Tool or ClustalW or any other suitable program which is suitable to generate sequence alignments.

Once the determination was done, the skilled person can then judge as to whether there is a difference when comparing the “query sequence” with the “reference sequence”; see the above two paragraphs [0031] and [0032].

A “gene” when used herein is, so to say, a species of a nucleotide sequence and comprises a coding sequence for a polypeptide (here any of the Influenza genes described herein) and, optionally a 5′-UTR (containing, for example, expression control elements such as a promoter) and/or 3′-UTR (containing, for example, a termination signal sequence). The gene may be composed of exons and introns or may be free of introns, thus merely composed of exons. It may be composed of DNA, genomic DNA, cDNA, and in case of Infleunza virus a gene may be composed of vRNA or cRNA. Usually, a gene comprises an open reading frame (ORF) that starts with the start codon “ATG” encoding the amino acid methionine (Met). Thus, when reference is made herein to a “gene”, it is preferably envisaged that the term “gene” is interchangeably used with the term “ORF”. Put differently, when reference to gene is made, it is preferred that the ORF comprised by that gene is meant. For example, in case of SEQ ID No: 1 the ORF starts at position 46 of the nucleotide sequence. Accordingly, when reference to the gene shown in SEQ ID No: 1 is made herein and the ORF is preferably meant, 45 nucleotides have to be subtracted from all nucleotide positions mentioned herein in relation to SEQ ID No: 1.

When used herein, the term “polypeptide” or “protein” (both terms are used interchangeably herein) means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The terms polypeptide and protein are often used interchangeably herein. The term polypeptide also refers to, and does not exclude, modifications of the polypeptide, e.g., glycosylation, acetylation, phosphorylation and the like. Such modifications are well described in basic texts and in more detailed monographs, as well as in the research literature. Generally, the skilled person knows, because of his common general knowledge and the context when terms such as NP, HA, PA, PB1, PB2, NS are used, as to whether the nucleotide sequence or nucleic acid, or the amino acid sequence or polypeptide, respectively, is meant.

A live, attenuated influenza A virus according to the present invention, including, e.g. a respective virus in a pharmaceutical composition, may be based on any influenza A virus such as a bird flu, human flu, swine influenza, equine influenza or a canine influenza. Various different influenza A virus subtypes exist, differing in the nature of the HA and NA glycoproteins on their surface. Influenza A viruses are accordingly usually categorized into subtypes based on the combination of protein forms of Hemagglutinin and Neuraminidase present, two proteins on the surface of the viral envelope. Sixteen Hemagglutinin forms (H1 to H16) and nine Neuraminidase forms (N1 to N9) have been identified.

Suitable virus strains include, but are not limited to H1N1, H1N2, H1N3, H1N4, H1N5, H1N6, H1N7, H1N8, H1N9, H2N1, H2N2, H2N3, H2N4, H2N5, H2N6, H2N7, H2N8, H2N9, H3N1, H3N2, H3N3, H3N4, H3N5, H3N6, H3N7, H3N8, H3N9, H4N1, H4N2, H4N3, H4N5, H4N6, H4N7, H4N8, H5N1, H5N2, H5N3, H5N4, H5N6, H5N7, H5N8, H5N9, H6N1, H6N2, H6N3, H6N4, H6N5, H6N7, H6N8, H6N9, H7N1, H7N2, H7N3, H7N4, H7N5, H7N6, H7N7, H7N8, H7N9, H8N1, H8N2, H8N3, H8N4, H8N5, H8N6, H8N7, H9N1, H9N2, H9N3, H9N4, H9N5, H9N6, H9N7, H9N8, H9N9, H10N1, H10N2, H10N3, H10N4, H10N5, H10N6, H10N7, H10N8, H10N9, H11N1, H11N2, H11N3, H11N4, H11N5, H11N6, H11N7, H11N8, H11N9, H12N1, H12N2, H12N3, H12N4, H12N5, H12N6, H12N7, H12N8, H12N9, H13N1, H13N2, H13N3, H13N4, H13N5, H13N6, H13N8, H14N3, H14N5, H14N6, H15N8, H15N9, H16N3. In some embodiments the influenza virus is one of the strains H1N1, H1N2, H2N2, H3N1, H3N2, H5N1 and H7N7.

An example of a H1N1 strain is Influenza A virus strain A/Puerto Rico/8/1934(H1N1) with Gene bank accession number NC 002016, NC 002017, NC 002018, NC 002019, NC 002020, NC 002021, NC 002022, NC 002023. Further examples of a H1N1 strain are Influenza A strain A/Brevig Mission/1/1918 H1N1) (Influenza A virus (strain A/South Carolina/1/1918 H1N1), Influenza A strain A/Russia:St.Petersburg/8/2006 H1N1, Influenza A strain A/USA:Texas/UR06-0195/2007 H1N1-strain A/Brevig Mission/1/1918 H1N1, Influenza A strain A/South Carolina/1/1918 H1N1, Influenza A strain A/Swine/lowa/15/1930 H1N1, Influenza A strain A/Wilson-Smith/1933 H1N1, Influenza A strain A/WS/1933 H1N1, and strain A/USA:Phila/1935 H1N1. A further example of a H1N1 strain is Influenza A virus strain A/New Zealand:South Canterbury/35/2000 H1N1. An example of a H1N2 strain is Influenza A virus strain A/Xianfeng/3/1989 H1N2. Two examples of a H1N3 strain are Influenza A/duck/NZL/160/1976 H1N3 and strain A/Whale/Pacific ocean/19/1976 H1N3. An example of a H1N4 strain is Influenza A virus strain A/mallard/Netherlands/30/2006 H1N4. An example of a H1N5 strain is Influenza A virus strain A/pintail duck/ALB/631/1981 H1N5. An example of a H1N6 strain is Influenza A virus strain A/murre/Alaska/305/1976 H1N6. An example of a H1N7 strain is Influenza A virus A/swine/England/191973/92 H1N7. An example of a H1N8 strain is strain A/Egyptian goose/South Africa/AI1448/2007. An example of a H2N1 strain is Influenza A virus strain A/Japan/Bellamy/57 H2N1. An example of a H2N2 strain is Influenza A virus strain A/Korea/426/68 H2N2 with Gene bank accession numbers NC 007366, NC 007367, NC 007368, NC 007369, NC 007370, NC 007374, NC 007375, NC 007376, NC 007377, NC 007378, NC 007380, NC 007381 and NC 007382. Three further examples of a H2N2 strain are Influenza A strain A/Japan/305/1957 H2N2, A/Czech Republic/1/1966 H2N2 and strain A/Singapore/1/1957 H2N2. An example of a H2N3 strain is Influenza A virus strain A/mallard/Minnesota/Sg-00692/2008 H2N3. An example of a H2N4 strain is A/mallard/Alberta/149/2002 H2N4. An example of a H2N5 strain is Influenza A virus strain A/tern/Australia/1/04 H2N5. An example of a H2N6 strain is Influenza A virus strain A/thick-billed murre/Alaska/44145-199/2006 H2N6. An example of a H2N7 strain is Influenza A virus strain A/northern shoveler/California/HKWF1128/2007 H2N7. An example of a H2N8 strain is Influenza A virus strain A/turkey/CA/1797/2008 H2N8. An example of a H2N9 strain is Influenza A virus strain A/duck/Germany/1972 H2N9. An example of a H3N1 strain is Influenza A virus strain A/mallard duck/ALB/26/1976 H3N1. An example of a H3N2 strain is Influenza A virus strain A/New York/392/2004 H3N2 with Gene bank accession numbers NC 007371, NC 007372 and NC 007373. Five further example of a H3N2 strain are Influenza A virus strain NX-31 H3N2, strain A/Hong Kong/5/1983 H3N2, A/Rio/6/69 H3N2, A/Hong Kong/MA/1968 H3N2 and Influenza A virus strain A/Shanghai/N12/2007 H3N2. An example of a H3N3 strain is Influenza A virus strain A/duck/Hong Kong/22A/1976 H3N3. An example of a H3N4 strain is Influenza A virus strain A/mallard duck/ALB/1012/1979 H3N4. An example of a H3N5 strain is Influenza A virus strain A/northern shoveler/California/HKWF1046/2007 H3N5. An example of a H3N6 strain is Influenza A virus strain A/Chicken/Nanchang/9-220/2000 H3N6. Examples of a H3N8 strain are Influenza A strain A/Equine/Miami/1/1963 H3N8 and strain A/Duck/Ukraine/1/1963 H3N8. An example of a H3N9 strain is Influenza A virus strain A/swan/Shimane/227/01 H3N9.

An example of a H4N1 strain is Influenza A virus strain A/chicken/Singapore/1992(H4N1). An example of a H4N2 strain is Influenza A virus strain A/duck/Hong Kong/24/1976(H4N2). An example of a H4N3 strain is Influenza A virus strain A/mallard/Sweden/65/2005(H4N3). An example of a H4N4 strain is Influenza A virus strain A/Grey teal/Australia/2/1979 H4N4. An example of a H4N5 strain is Influenza A virus strain A/duck/Hokkaido/1058/2001(H4N5). Two examples of a H4N6 strain are Influenza A virus strain A/Duck/Czechoslovakia/1956 H4N6 and Influenza A virus strain A/Duck/Alberta/28/1976 H4N6. An example of a H4N7 strain is Influenza A virus strain A/duck/Mongolia/583/02 H4N7. An example of a H4N8 strain is Influenza A virus strain A/Chicken/Alabama/1/1975 H4N8. An example of a H4N9 strain is Influenza A virus strain A/WDk/ST/988/2000(H4N9). An example of a H5N1 strain is Influenza A virus (A/Goose/Guangdong/1/96(H5N1)) with Gene bank accession numbers NC 007357, NC 007358, NC 007359, NC 007360, NC 007362, NC 007363, and NC 007364. Further examples of a H5N1 strain are Influenza A strain A/Duck/Hong Kong/2986.1/2000 H5N1, Influenza A strain A/Silky Chicken/Hong Kong/SF189/2001 H5N1, Influenza A strain A/Chicken/Hong KongNU562/2001 H5N1, Influenza A strain A/Chicken/Hong Kong/FY150/2001 H5N1, Influenza A strain A/Chicken/Hong Kong/715.5/2001 H5N1, Influenza A strain A/Guinea fowl/Hong Kong/38/2002 H5N1, Influenza A strain A/Chicken/Hong Kong/31.2/2002 H5N1, Influenza A strain A/Chicken/Hong Kong/37.4/2002 H5N1, Influenza A strain A/Silky Chicken/Hong KongNU100/2002 H5N1, Influenza A strain A/Chicken/Hong Kong/96.1/2002 H5N1, Influenza A strain A/Chicken/Hong KongNU22/2002 H5N1, Influenza A strain A/Teal/China/2978.1/2002 H5N1, Influenza A strain A/Hong Kong/212/2003 H5N1, Influenza A strain A/Chicken/Shantou/4231/2003 H5N1, and Influenza A strain A/Goose/Guangxi/345/2005 H5N1. An example of a H5N2 strain is Influenza A strain A/Chicken/Pennsylvania/1370/1983 H5N2. An example of a H5N3 strain is Influenza A strain A/duck/Malaysia/F119-3/97 H5N3. An example of a H5N4 strain is Influenza A strain A/environment/New York/200269-18/2002 H5N4. An example of a H5N5 strain is Influenza A strain A/duck/Massachusetts/Sg-00440/2005 H5N5. An example of a H5N6 strain is A/duck/Potsdam/2216-4/1984 H5N6. An example of a H5N7 strain is A/mallard/Denmark/64650/03 H5N7. An example of a H5N8 strain is strain A/Duck/Ireland/113/1983 H5N8. Two examples of a H5N9 strain are Influenza A strain A/Turkey/Ontario/7732/1966 H5N9 and strain A/chicken/Italy/22AM 998 H5N9.

An example of a H6N1 strain is A/chicken/Taiwan/PF1/02(H6N1). An example of a H6N2 strain is Influenza A strain A/chicken/California/1316/2001(H6N2). An example of a H6N5 strain is Influenza A strain A/Shearwater/Australia/1972 H6N5. An example of a H6N8 strain is Influenza A strain A/Turkey/Minnesota/501/1978 H6N8. An example of a H7N1 strain is Influenza A strain A/Fowl plague virus/Rostock/8/1934 H7N1. An example of a H7N2 strain is Influenza A strain A/duck/Hong Kong/293/1978(H7N2). An example of a H7N3 strain is Influenza A strain strain A/Turkey/Oregon/1971 H7N3. Five examples of a H7N7 strain are Influenza A strain A/Equine/C.Detroit/1/1964 H7N7, Influenza A strain A/Equine/Cambridge/1/1973 H7N7 and Influenza A strain A/Equine/Sao Paulo/1/1976 H7N7, Influenza A virus strain A/Equine/Prague/1/1956 H7N7 and Influenza A virus strain A/Chicken/Weybridge H7N7. An example of a H8N2 strain is Influenza A strain A/duck/Alaska/702/1991(H8N2). An example of a H8N4 strain is Influenza A strain A/Turkey/Ontario/6118/1968 H8N4. An example of a H8N4 strain is Influenza A strain A/duck/Tsukuba/255/2005(H8N5). An example of a H8N7 strain is Influenza A strain A/duck/Alaska/702/1991(H8N7).

An example of a H9N1 strain is Influenza A virus A/Duck/Shantou/2030/00(H9N1). An example of a H9N2 strain is Influenza A virus A/Hong Kong/1073/99(H9N2) with Gene bank accession numbers NC 004905, NC 004906, NC 004907, NC 004908, NC 004909, NC 004910, NC 004911, and NC 004912. An example of a H9N3 strain is Influenza A virus A/duck/Viet Nam/340/2001 H9N3. An example of a H9N4 strain is Influenza A virus A/shorebird/DE/231/2003 H9N4. An example of a H9N5 strain is Influenza A virus A/Duck/Hong Kong/702/79 H9N5. An example of a H9N7 strain is A/turkey/Scotland/70(H9N7). An example of a H9N8 strain is A/chicken/Korea/04164/2004(H9N8). An example of a H9N9 strain is A/turkey/France/03295/2003 H9N9. An example of a H10N1 strain is Influenza A virus A/duck/Hong Kong/938/80 H10N1. An example of a H10N2 strain is Influenza A virus A/duck/Alaska/658/1991 H10N2. An example of a H10N5 strain is Influenza A virus A/duck/Hong Kong/15/1976 H10N5. Examples of a H10N7 strain are Influenza A strain A/Chicken/Germany/n/1949 H10N7, strain A/Duck/Germany/1949 H10N7, and strain A/Duck/Manitoba/1/1953 H10N7. An example of a H10N7 strain is Influenza A virus strain A/Duck/Germany/1949 H10N7. An example of a H11N1 strain is Influenza A virus strain A/duck/Miyagi/47/1977 H11N1. An example of a H11N2 strain is A/duck/Yangzhou/906/2002 H11N2. An example of a H11N3 strain is A/duck/Thailand/CU5388/2009 H11N3. An example of a H11N6 strain is Influenza A virus strain A/Duck/England/1/1956 H11N6. An example of a H11N8 strain is strain A/Duck/Ukraine/2/1960 H11N8. Two examples of a H11N9 strain are Influenza A strain A/Duck/Ukraine/1/1960 H11N9 and Influenza A strain A/Tern/Australia/G70C/1975 H11N9. An example of a H12N1 strain is A/mallard duck/Alberta/342/1983(H12N1). An example of a H12N2 strain is A/duck/Primorie/3691/02 H12N2. An example of a H12N3 strain is A/whooper swan/Mongolia/232/2005 H12N3. An example of a H12N5 strain is Influenza A virus strain A/Duck/Alberta/60/1976 H12N5. An example of a H12N6 strain is A/mallard/Alberta/221/2006 H12N6. An example of a H12N7 strain is A/duck/Victoria/30a/1981 H12N7. An example of a H12N8 strain is A/mallard/Netherlands/20/2005 H12N8. An example of a H12N9 strain is A/red-necked stint/Australia/5745/1981 H12N9.

An example of a H13N1 strain is A/bird feces/Illinois/185997-30/2007 H13N1. An example of a H13N2 strain is Influenza A virus strain A/Whale/Maine/328/1984 H13N2. An example of a H13N3 strain is A/shorebird/NJ/840/1986 H13N3. Two examples of a H13N6 strain are Influenza A virus strain A/Gull/Maryland/704/1977 H13N6 and strain A/Gull/Minnesota/945/1980 H13N6. An example of a H13N8 strain is A/black-headed gull/Sweden/1/2005 H13N8. An example of a H14N3 strain is A/mallard/Gur/263/82 H14N3. Three examples of a H14N5 strain are A/mallard/Gurjev/263/1982 H14N5, A/mallard/Astrakhan/266/1982 H14N5 and A/herring gull/Astrakhan/267/1982 H14N5. An example of a H14N6 strain is strain A/Mallard/Gurjev/244/1982 H14N6. An example of a H15N8 strain is A/duck/Australia/341/1983 H15N8. An example of a H15N9 strain is A/shearwater/West Australia/2576/79 H15N9. An example of a H16N3 strain is A/black-headed gull/Sweden/2/99 H16N3.

Such virus subtypes are distinguishable serologically, which means that antibodies specific for one subtype do not bind to another subtype with comparable high affinity. Nevertheless the nucleic acid positions characterizing the genes of an Influenza A virus according to the present invention apply to any Influenza A virus strain.

A live, attenuated Influenza A virus according to the invention has a silent mutation at one or more of the nucleotide positions that are located 19, 22, 25, 28, 31, 40, 43, 70 and 76 nucleotides from the end of the stop codon at the 3′-end of the sequence encoding the nucleoprotein of Influenza A virus. These positions correspond to nucleotides 1524, 1521, 1518, 1515, 1512, 1503, 1500, 1473 and 1467 of numerous data base entries of the Influenza A nucleoprotein, inter alia, the nucleic acid sequence of the nucleoprotein of Influenza A strain A/Puerto Rico/8/1934 H1N1 (EMBL-Bank accession Nos J02147 or M38279), of strain A/Ohio/4/1983 H1N1 (EMBL-Bank accession No M59334), strain A/Victoria/5/1968 H2N2 (EMBL-Bank accession No M63753) or, in particular strain A/FPV/Rostock/1934 H7N1 (SEQ ID No: 1, EMBL-Bank accession No M21937) is used as reference sequence. As an alternative reference sequence to SEQ ID No: 1, the nucleotide sequence shown in SEQ ID NO. 47 can be used (strain A/WSN/33). If so, 45 nucleotides have to be subtracted from the nucleotide positions mentioned in the context of a nucleotide position in (or of) the nucleotide sequence shown in SEQ ID No: 1. In some embodiments the Influenza A virus according to the invention has a silent mutation at two, three, four, five, six, seven, eight or nine of the nucleotide positions that correspond to nucleotides 1524, 1521, 1518, 1515, 1512, 1503, 1500, 1473 and 1467 within the NP gene shown in SEQ ID No: 1. It is understood that numerous mutations—some of them silent—exist and occur in the Influenza A virus, many of them giving rise to new variants and strains and causing influenza outbreaks. Hence, in addition to the above silent mutations at one or more of positions 1524, 1521, 1518, 1515, 1512, 1503, 1500, 1473 and 1467 of the NP gene, an Influenza A virus according to the invention may have further mutations relative to the sequence of SEQ ID No: 1. Typically the NP-gene of an Influenza A virus according to the invention nevertheless encodes a NP polypeptide, in particular a functional nucleoprotein. By an “Influenza virus gene” when used herein is generally meant the corresponding influenza virus open reading frame. Typically an influenza virus gene is a full-length gene. In other embodiments, a gene fragment is used that includes about 60%, about 70%, about 75%, about 80%, about 85%, about 90%, about 92%, about 95%, about 97%, about 98% or about 99% of a full-length gene.

In embodiments where more than two, in particular six, seven, eight or nine of the nucleotide positions that correspond to nucleotides 1524, 1521, 1518, 1515, 1512, 1503, 1500, 1473 and 1467 of the NP gene shown in SEQ ID No: 1 have a silent mutation the respective influenza virus has an extremely low risk of back mutation to an infectious influenza virus. With each silent mutation present the risk of such a back mutation exponentially decreases. This risk can be even further reduced by including one or more silent mutations in the PA gene of the influenza A virus, as explained below. An influenza A virus according to the invention can thus provide a highly stable and thereby secure live vaccine.

In general, the term “fragment”, as used herein with respect to an Influenza virus according to the disclosure, relates to shortened nucleic acid or amino acid sequences that correspond to a certain Influenza virus but lack a portion thereof. They may, for example, be an N-terminally and/or C-terminally shortened sequence, of which a nucleic acid sequence retains the capability of being expressed and of which an amino acid sequence retains the capability of being recognized and/or bound by an immunoglobulin in a mammalian or avian body.

A silent mutation is a mutation of a nucleic acid sequence that does not affect the protein sequence that is encoded from the respective nucleic acid sequence. Hence, such a mutation on the nucleic acid level alters a codon, coding for a certain amino acid, to another codon that codes for the same amino acid. A silent mutation according to the present invention may also be called a “synonymous substitution”, since the silent mutations defined herein are located within a coding region. A silent mutation can occur due to the redundancy or degeneracy of the genetic code, meaning that a number of amino acids are encoded by two, three, four or six different base triplets. Typically a silent mutation is defined by an exchange in the third base of the triplet of a codon. As a few examples, the amino acid glycine is encoded by the RNA triplets GGU, GGC, GGA and GGG, the amino acid arginine is encoded by the RNA triplets AGA, AGG, CGU, CGC, CGA and CGG, the amino acid leucine is encoded by the RNA triplets UUA, UUG, CUU, CUC, CUA and CUG, the amino acid threonine is encoded by the RNA triplets ACU, ACC, ACA and ACG and the amino acid alanine is encoded by the RNA triplets GCU, GCC, GCA and GCG. As three further examples, the amino acid serine is encoded by the RNA triplets AGU, AGC, UCU, UCC, UCA and UCG, the amino acid isoleucine is encoded by the RNA triplets AUU, AUC and AUA and the amino acid valine is encoded by the RNA triplets GUU, GUC, GUA and GUG. As can be seen from these examples, an exchange of a single nucleic acid within a triplet can result in the same amino acid being encoded. For instance replacing the third base cytosine in the codon GCC with the base adenine generates the triplet GCA. Both of these codons are translated into the amino acid alanine so that no change on the amino acid level is caused.

In some embodiments a live, attenuated Influenza A virus according to the invention has an adenine at position 1467 of SEQ ID No: 1, while in other embodiments it has a cytosine at position 1467, and in yet further embodiments it has an uracil at position 1467. It is understood that these indications refer to the RNA sequence, as present in the virus in vivo. In the corresponding sequence of deoxyribonucleotides of a DNA sequence thymine is present, rather than uracil. In some embodiments the Influenza A virus according to the invention has a guanine at position 1467. In some embodiments a live, attenuated Influenza A virus according to the invention has an adenine at position 1473, while in other embodiments it has a cytosine at position 1473 of SEQ ID No: 1, and in yet further embodiments it has an uracil at position 1473. In some embodiments the Influenza A virus according to the invention has a guanine at position 1473. In some embodiments an Influenza A virus according to the invention has a cytosine at position 1500, while in other embodiments it has an adenine at position 1500, and in yet further embodiments it has an uracil at position 1500 of SEQ ID No: 1. In some embodiments the Influenza A virus according to the invention has a guanine at position 1500. A live, attenuated Influenza A virus according to the invention has in some embodiments a cytosine at position 1503 of SEQ ID No: 1, while in other embodiments it has an adenine at position 1503, and in yet further embodiments it has an guanine at position 1503. In some embodiments the Influenza A virus according to the invention has an uracil at position 1503. In some embodiments a live, attenuated Influenza A virus according to the invention has an uracil at position 1512 of SEQ ID No: 1. In some embodiments the Influenza A virus according to the invention has a cytosine at position 1512 of SEQ ID No: 1. A live, attenuated Influenza A virus according to the invention has in some embodiments a cytosine at position 1515 of SEQ ID No: 1, while in other embodiments it has an uracil at position 1515, and in yet further embodiments it has an guanine at position 1515 of SEQ ID No: 1. In some embodiments the Influenza A virus according to the invention has an adenine at position 1515 of SEQ ID No: 1. In some embodiments a live, attenuated Influenza A virus according to the invention has an uracil at position 1518 of SEQ ID No: 1. In some embodiments the Influenza A virus according to the invention has a cytosine at position 1518 of SEQ ID No: 1. In some embodiments a live, attenuated Influenza A virus according to the invention has a cytosine at position 1521 of SEQ ID No: 1. In some embodiments the Influenza A virus according to the invention has an uracil at position 1521. In some embodiments a live, attenuated Influenza A virus according to the invention has a cytosine at position 1524 of SEQ ID No: 1, while in other embodiments it has a guanine at position 1524, and in yet further embodiments it has an uracil at position 1524 of SEQ ID No: 1. In some embodiments the Influenza A virus according to the invention has an adenine at position 1524 of SEQ ID No: 1.

In some embodiments a live, attenuated Influenza A virus according to the invention may have, for example in addition to one or more of the above silent mutations, a silent mutation in the PA gene, which encodes the polymerase PA. These silent mutations are located at nucleotide positions that correspond to nucleotides 2100 and/or 2103 of SEQ ID No: 3. In some embodiments a respective Influenza A virus according to the invention has a cytosine at position 2100. In some embodiments a live, attenuated Influenza A virus according to the invention has an adenine at position 2100. In some embodiments the Influenza A virus according to the invention has a guanine at position 2100. In some embodiments the Influenza A virus has no silent mutation at position 2100 and thus an uracil at this position. In some embodiments an Influenza A virus according to the invention has a adenine at position 2103. In some embodiments a live, attenuated Influenza A virus according to the invention has an adenine at position 2103, and accordingly no silent mutation at this position. An Influenza A virus according to the invention may have further mutations in addition to the above two silent mutations at one or both of positions 2100 and 2103 of the PA gene, relative to the sequence of SEQ ID No: 3. Typically the PA-gene of an Influenza A virus according to the invention nevertheless encodes a polymerase PA polypeptide, in particular a functional nucleoprotein.

When reference is made to a certain sequence, including a SEQ ID, an Influenza virus according to the invention includes a variant of a respective sequence. Generally, a variant of a respective sequence as disclosed herein comprises one or more of the silent mutations described herein. It is recalled in this regard that RNA viruses have notoriously high mutation rates due to the error prone nature of the viral polymerase. This mutation rate causes the antigenic drift, which in turn gives rise to repeated global pandemics. A respective “variant” means a biologically active nucleic acid sequence that has at least about 70%, including at least about 80% or at least about 85%, at least about 90%, at least about 92%, at least about 95% or at least about 98% base sequence identity with the sequence to which reference is made, for example a native Influenza virus strain or a mutant thereof. On the amino acid level a respective variant may have at least about 70%, including at least about 80%, at least about 85%, at least about 90%, at least about 92%, at least about 95%, at least about 96%, at least about 97%, at least about 98%, at least about 99% or at least about 99.5% amino acid sequence identity with the sequence to which reference is made, for example a native Influenza virus strain or a mutant thereof. Such variants include, for instance, polypeptides in which one or more amino acid residues are added or deleted at the N- or C-terminus of the polypeptide. “Biologically active” in the context of a variant means that such a variant virus is at least capable of eliciting an immune response.

“Percent (%) sequence identity” with respect to nucleotide acid sequences disclosed herein is defined as the percentage of nucleotide residues in a candidate sequence that are identical with nucleotide residues in a reference sequence, i.e. an attenuated Influenza virus nucleotide sequence of the present disclosure, after aligning the sequences and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publically available computer software such as BLAST, ALIGN, or Megalign (DNASTAR) software. Those skilled in the art can determine appropriate parameters for measuring alignment, including any algorithms needed to achieve maximum alignment over the full length of the sequences being compared. The same embodiment is equally applicable for “percent (%) sequence identity” with respect to amino acid sequences disclosed herein, mutatis mutandis, i.e., each of the amino acid sequences disclosed herein can serve as reference sequence when being compared with a query sequence in order to determine the percent value of sequence identity between the reference and the query sequence.

An Influenza virus according to the present invention may be an enriched, isolated and/or purified virus, isolated and/or purified by means of in vitro preparation, so that it is not associated with in vivo compounds or other substances, or is at least substantially purified from in vitro substances. An isolated virus preparation according to the invention is generally obtained by in vitro culture and propagation and is substantially free from other infectious agents. For example, “isolated” when used in relation to a polypeptide or nucleic acid, as in “isolated protein”, “isolated polypeptide” or “isolated nucleic acid” refers to a polypeptide or nucleic acid, respectively, that is identified and separated from at least one contaminant with which it is ordinarily associated in its natural source.

The term “purified” as used herein refers to material that has been isolated under conditions that reduce or eliminate the presence of unrelated materials, i.e. contaminants, including native materials from which the material is obtained. A purified virus is for example typically substantially free of host cell or culture components, including tissue culture or egg proteins or non-specific pathogens. As used herein, “substantially free” means below the level of detection for a particular infectious agent using standard detection methods for that agent. Similarly, a “purified nucleic acid” or “purified polypeptide” refers to a nucleic acid or polypeptide, respectively, that is essentially free from other contaminants

A “recombinant” virus is a virus that has been manipulated in vitro, e.g., using genetic engineering techniques well known in the art to introduce changes to the viral genome. Typically purified material substantially free of contaminants is at least 50% pure, such as, at least 90% pure or at least 99% pure. Purity can be evaluated by chromatography, gel electrophoresis, immunoassay, composition analysis, biological assay, and other methods known in the art. Viral particles can for instance be purified by ultrafiltration through sucrose cushions or by ultracentrifugation, such as continuous centrifugation.

In some embodiments an attenuated Influenza A virus according to the invention is combined with a further Influenza A virus and one Influenza B virus. In some of these embodiments the further Influenza A virus is likewise an attenuated Influenza A virus according to the invention. In some of these embodiments the Influenza B virus is an attenuated Influenza B virus. This attenuated Influenza B virus may include an NP gene with one or more silent mutations in its C-terminal region.

A composition according to the inventions may include a live, attenuated influenza A virus as defined above. Such a composition may be for use in the immunization of a mammal such as a human or for use in the immunization of a bird. In some embodiments a respective bird or mammal, including a human, is immunocompromised.

There are number of problems with the currently marketed seasonal flu vaccines. First, yearly adaptation of strains is required according to forecast by WHO, with a short window to immunize target populations. This is especially true for young children, who require two doses for priming. The standard vaccine, killed trivalent-split/subunit (TIV), is poorly priming in children. That is, it induces no or only weak immunity in naïve individuals, which is suspected to compromise induction of cross-immunity to other strains/subtypes and thus being counterproductive to the immune constitution (Bodewes et al., Lancet 2009; 9: 784-8; Bodewes et al., PlosOne 2009; 9: e5538:1-9). Likewise, the only two available live cold-adapted influenza vaccines, Flumist™ and the influenza A (H1N1) 2009, cannot be used in elderly populations, and can only be used in healthy children of at least 24 months of age.

In contrast thereto, an influenza virus according to the present invention is envisaged to be a “universal vaccine” both for seasonal and pandemic flu which would be able to protect against different influenza strains. Data obtained using mammals show that an influenza virus according to the invention can be used to form a vaccine that provides, immediately or several weeks after administration, protection to a host against doses of virus that are several fold, including one or two magnitudes, above the dose that is lethal to a non-protected host. In some embodiments a composition that includes an IAV virus as defined above confers protection to a mammal or bird against a 10-100-fold lethal dose of an IAV virus, which does not have any of the above defined silent mutations. Such mammal or bird has typically been administered at least one dose of about 10⁴ to about 10⁵ plaque forming units (PFU)/kg of the IAV virus.

Further, an influenza virus according to the present invention is envisaged to provide effective protection against influenza infection even in severely immunosuppressed organisms, such as birds or mammals, including humans. In addition, preliminary data suggest that in embodiments where more than two, in particular six, seven, eight or more (e.g. nine or more) of the nucleotide positions within an influenza virus genome disclosed herein have a silent mutation the respective influenza virus an serve as a particularly effective vaccine even in an immunocompromised host.

In this regard a composition that includes an IAV virus as defined above confers a high hemagglutinin inhibition (HI) titer to serum of a mammal or a bird. In some embodiments a composition that includes such an IAV virus confers to a serum sample from a mammal or from a bird a hemagglutinin inhibition (HI) titer of preferably at least about 1:520 when tested against the same IAV that does not have any of the above defined silent mutations. Such mammal or bird has typically been administered at least one dose of about 10⁴ to about 10⁵ plaque forming units (PFU)/kg of the IAV virus. The hemagglutinin inhibition (HI) titer may also be lower than about 1:520, such as about 1:512, about 1:256, about 1:128, about 1:64, about 1:32, about 1:16, about 1:8, about 1:4 or about 1:2. However, said titer is more preferably higher than 1:520, such as 1:1024 or 1:2048 or 1:4096 or 1:8192. The hemagglutin inhibition assay is described and preferably performed as in Katz et al. (2009. Vaccine Morbid. Mortal. Weekly Rep., 58 (19), 521-524 or Potter & Oxford (1979) Br Med Bull, 35, 69-75. A particular preferred hemagglutin inhibition assay is described in the appended Examples.

The invention also provides a live, attenuated influenza B virus (IBV). An IBV according to the present invention, including e.g. a respective virus in a pharmaceutical composition, may be based on any influenza B virus strain. Suitable virus strains include, but are not limited to Influenza B virus strain B/Maryland/1959, strain B/Yamagata/1/1973, strain B/Victoria/3/1985, strain B/USSR/100/1983, strain B/Tokyo/942/1996, strain B/Texas/4/1990, strain B/Singapore/222/1979, strain B/South Dakota/5/1989, strain B/Paris/329/1990, strain B/Leningrad/179/1986, strain B/Hong Kong/8/1973, strain B/Fukuoka/80/1981, strain B/Bangkok/163/1990, strain B/Beijing/1/1987, strain B/Switzerland/9359/99, strain B/Wisconsin/6/2006, strain B/West Virginia/01/2009, strain B/Washington/08/2009, strain B/Uruguay/NG/02, strain B/Texas/18/2001, strain B/Taiwan/S117/2005, strain B/Taiwan/3799/2006, strain B/Spain/WV45/2002, strain B/Seoul/232/2004, strain B/Rio Grande do Sul/57/2008, strain B/Quebec/517/98, strain B/Philippines/5072/2001, strain B/Oslo/1871/2002, strain B/Osaka/983/1997, strain B/Milan/05/2006, strain B/Johannesburg/116/01 or strain B/Arizona/12/2003.

A live, attenuated Influenza B virus according to the invention may have a silent mutation at one or more nucleotide positions of the sequences of the PB1 gene, encoding the polymerase catalytic subunit Polymerase basic protein 1, the PB2 gene, encoding the polymerase catalytic subunit Polymerase basic protein 2, the PA gene, encoding the Polymerase acidic protein, the HA gene, encoding Hemagglutinin, the NP gene, encoding the Nucleoprotein, the NA gene, encoding Neuraminidase, the M1 gene, encoding Matrix protein 1, the BM2 gene, encoding Influenza B Matrix protein 2 (BM2), the NS1 gene, encoding the Non-structural protein 1, and/or the NS2 gene, encoding the Non-structural protein NS2.

The nucleotide positions of the sequences of the PB1 gene are one or more, such as two, three, four, five, six or seven of the nucleotide positions corresponding to nucleotides 57 (PB1-A1), 60 (PB1-A2), 63 (PB1-A3), 66 (PB1-A4), 69 (PB1-A5), 2148 (PB1-A6) and 2154 (PB1-A7) of SEQ ID No: 5.

The nucleotide positions of the sequences of the PB2 gene are one or more, for instance two, three, four, five, six or seven of the nucleotide positions corresponding to nucleotides 93 (PB2-A1), 96 (PB2-A), 99 (PB2-A3), 102 (PB2-A4), 2283 (PB2-A5), 2286 (PB2-A6) of SEQ ID No: 7.

The nucleotide positions of the sequences of the PA gene are one or more, such as two, three, four, five, six, seven, eight, nine, ten or eleven of the nucleotide positions corresponding to nucleotides nucleotide 33 (PA-A1), 36 (PA-A2), 39 (PA-A3), 42 (PA-A4), 45 (PA-A5), 57 (PA-A6), 60 (PA-A7), 66 (PA-A8), 69 (PA-A9), 2175 (PA-A10), and 2178 (PA-A11) of SEQ ID No: 9.

A silent mutation in the HA gene of the live, attenuated Influenza B virus is at one or more, for instance two, three or four of the nucleotide positions that corresponds to nucleotides 57 (HA-A1), 60 (HA-A2), 1608 (HA-A3) and 1611 (HA-A4) of SEQ ID No: 11.

As an example, in some embodiments a live, attenuated Influenza B virus according to the invention has an uracil at position 57 of SEQ ID No: 11. In some embodiments the Influenza B virus according to the invention has a cytosine at position 57 of SEQ ID No: 11, and thus no silent mutation at this position. In some embodiments a live, attenuated Influenza B virus according to the invention has a cytosine at position 60 of SEQ ID No: 11. In some embodiments a live, attenuated Influenza B virus according to the invention has a cytosine at position 60 of SEQ ID No: 11. In some embodiments a live, attenuated Influenza B virus according to the invention has an adenine at position 60 of SEQ ID No: 11. In some embodiments a live, attenuated Influenza B virus according to the invention has a guanine at position 60 of SEQ ID No: 11. In some embodiments a live, attenuated Influenza B virus according to the invention has an uracil, and thus no silent mutation at this position.

As yet a further example, in some embodiments the Influenza B virus according to the invention has a cytosine at position 1608 of SEQ ID No: 11. In some embodiments an Influenza B virus according to the invention has a guanine at position 1608 of SEQ ID No: 11. In some embodiments a live, attenuated Influenza B virus according to the invention has an uracil at position 60 of SEQ ID No: 11. In some embodiments the Influenza B virus according to the invention has an adenine at position 57 of SEQ ID No: 11, and thus no silent mutation at this position.

The nucleotide positions of the sequences of the NP gene are one or more, such as two, three, four, five, six, seven, eight, nine, ten, eleven, twelve, thirteen, fourteen, fifteen, sixteen, seventeen, eighteen, nineteen, twenty or twenty-one of the nucleotide positions corresponding to nucleotides nucleotide 15 (NP-A1), 57 (NP-A2), 60 (NP-A3), 837 (NP-A4), 840 (NP-A5), 843 (NP-A6), 1572 (NP-A7), 1575 (NP-A8), 1578 (NP-A9), 1581 (NP-A10), 1584 (NP-A11), 1638 (NP-A12), 1641 (NP-A13), 1644 (NP-A14), 1647 (NP-A15), 1650 (NP-A16), 1653 (NP-A17), 1656 (NP-A18), 1659 (NP-A19), 1671 (NP-A20) and 1674 (NP-A21) of SEQ ID No: 13.

The nucleotide positions of the sequences of the NA gene are one or more, such as two, three, four or five of the nucleotide positions corresponding to nucleotides 255 (NA-A1), 258 (NA-A2), 1239 (NA-A3), 1242 (NA-A4) and 1245 (NA-A5) of SEQ ID No: 15.

The nucleotide positions of the sequences of the M1 gene are one or more, such as two, three, four, five, six, seven, eight, nine, ten, eleven or twelve of the nucleotide positions corresponding to nucleotides 12 (M1-A1), 15 (M1-A2), 18(M1-A3), 57(M1-A4), 60(M1-A5), 63(M1-A6), 705(M1-A7), 708 (M1-A8), 717 (M1-A9), 720 (M1-A10), 723 (M1-A11) and 726 (M1-A12) of SEQ ID No: 17. These nucleotide positions of the sequences of the M1 gene correspond to nucleotides 36, 39, 42, 81, 84, 87, 729, 732, 741, 744, 747 and 750 of the sequence of GenBank accession number J02094.

The nucleotide positions of the sequences of the BM2 gene are one or more, such as two or three of the nucleotide positions corresponding to nucleotides 147 (BM2-A1), 150 (BM2-A2) and 153 (BM2-A3) of SEQ ID No: 21. These nucleotide positions of the sequences of the BM2 gene correspond to nucleotides 897, 900 and 903 of the sequence of GenBank accession number DQ792900, and nucleotides 917, 920 and 923 of the sequence of GenBank accession number J02094.

The nucleotide positions of the sequences of the NS1 gene are one or more, such as two, three, four, five, six, seven, eight, nine, ten, eleven, twelve, thirteen, fourteen, fifteen, sixteen or seventeen of the nucleotide positions corresponding to nucleotides nucleotide 51 (NS1-A1), 54 (NS1-A2), 63 (NS1-A3), 66 (NS1-A4), 69 (NS1-A5), 687 (NS1-A6), 690 (NS1-A7), 693 (NS1-A8), 696 (NS1-A9), 762 (NS1-A10), 765 (NS1-A11), 768 (NS1-A12), 771 (NS1-A13), 774 (NS1-A14), 801 (NS1-A15), 804 (NS1-A16) and 807 (NS1-A17) of SEQ ID No: 19.

The nucleotide positions of the sequences of the NS2 gene are one or more, such as two, three, four or five of the nucleotide positions corresponding to nucleotides 351 (NS2-A1), 354 (NS2-A2), 357 (NS2-A3), 360 (NS2-A4) and 363 (NS2-A5) of SEQ ID No: 23.

An attenuated Influenza B virus according to the invention has at least one of the above silent mutations within the PB1 gene, the PB2 gene, the PA gene, the HA gene, the NP gene, the NA gene, the M1 gene, the BM2 gene, the NS1 gene and the NS2 gene. In some embodiments the attenuated Influenza B virus has two, three, four, five, six, seven, eight, nine, ten, 11, 12, 13, 14, 15, 16, 17, 18, 19, 20, 21, 22, 23, 24, 25, 26, 27, 28, 29, 30, 31, 36, 40, 42, 44, 45, 47, 49, 50, 55, 58, 61, 63, 65, 68, 70, 73, 76, 78, 80, 83, 84, 85, 86, 87, 88 or 90 of the above silent mutations within the genes selected from the PB1 gene, the PB2 gene, the PA gene, the HA gene, the NP gene, the NA gene, the M1 gene, the BM2 gene, the NS1 gene and the NS2 gene. In some embodiments the attenuated Influenza B virus has two or more, such as three, four, five, six or more, of the above silent mutations within two or more, such as two, three, four, five, six, seven, eight, nine or ten of the genes selected from the PB1 gene, the PB2 gene, the PA gene, the HA gene, the NP gene, the NA gene, the M1 gene, the BM2 gene, the NS1 gene and the NS2 gene.

Similar to an influenza A virus according to the invention (supra), in embodiments where more than two, in particular six, seven, eight or more of the above nucleotide positions within the influenza B virus genome have a silent mutation the respective influenza virus has an extremely low risk of back mutation to an infectious influenza virus. Hence an influenza B virus according to the invention can provide a highly stable and thereby secure live vaccine.

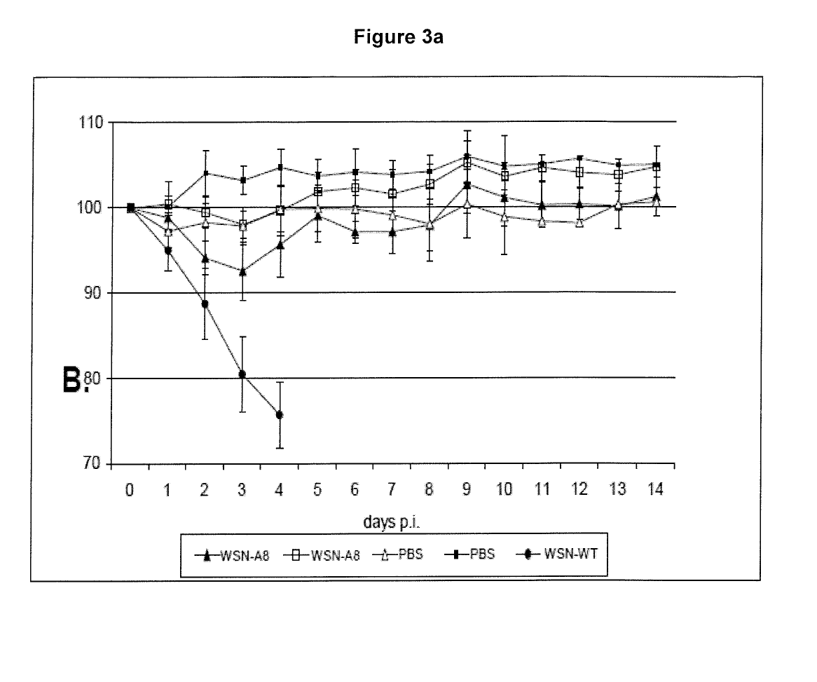

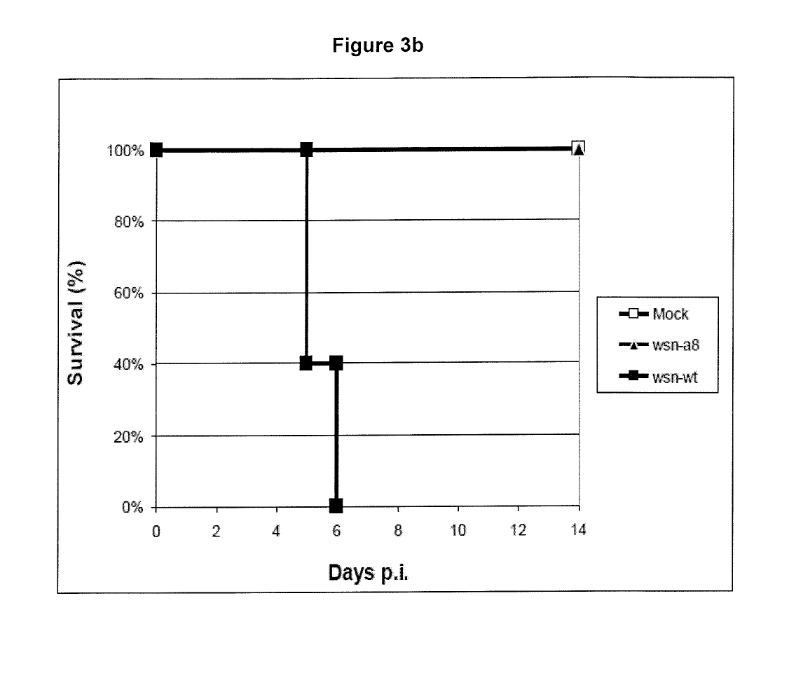

One of the silent mutations, i.e., NP-A7 that the present inventors found in the nucleotide sequence encoding the NP-protein of influenza virus, in particular influenza A virus, is at the third position of a codon coding for proline (Pro). Proline is unique among the natural amino acids. Unlike regular peptide bonds, the X-prolyl peptide bond will not adopt the intended conformation spontaneously, thus, the process of cis-trans isomerization can be the rate-limiting step in the process of protein folding. Prolyl isomerases therefore function as protein folding chaperones. Accordingly, without being bound by theory, the present inventors believe that a silent mutation in a codon encoding proline (Pro) in wild-type NP has an influence on the action of peptidyl prolyl isomerases (PPI) that in turn influences the folding of NP and, thus, the synthesis rate during translation, since they observed less NP in accordingly attenuated influenza viruses in comparison to wild-type viruses not having the NP-A7 mutation (see Anhlan et al., Vaccine (2012) FIG. 3B and 3C),

Hence, in another embodiment the present invention provides an attenuated influenza virus, preferably an influenza A virus, comprising a silent mutation at one or more positions corresponding to a position selected from nucleotide 1107 (P1), nucleotide 1275 (P2), nucleotide 1302 (P3), nucleotide 1404 (P4), nucleotide 1467 (P5), and nucleotide 1476 (P6) of SEQ ID No: 1.

Also provided is a influenza A virus NP gene having in its nucleotide sequence a silent mutation at one or more positions corresponding to a position selected from nucleotide 1107 (P1), nucleotide 1275 (P2), nucleotide 1302 (P3), nucleotide 1404 (P4), nucleotide 1467 (P5), and nucleotide 1476 (P6) of SEQ ID No: 1, as well as a vector and influenza virus comprising said gene.

It is more preferred that the attenuated influenza virus of the present invention, preferably the influenza A virus, that is preferably obtainable by the methods described herein, further comprises a silent mutation at one or more positions corresponding to a position selected from nucleotide 1107 (P1), nucleotide 1275 (P2), nucleotide 1302 (P3), nucleotide 1404 (P4), nucleotide 1467 (P5), and nucleotide 1476 (P6) of SEQ ID No: 1. Each of the P1, P2, P3, P4, P5 and P6 mutation is at the third position of a codon encoding proline. Proline is encoded by the codons CCA, CCG, CCT or CCC. Accordingly, in the context of the P1, P2, P3, P4, P5, P6 mutation(s), dependent on which codon is present in the NP nucleotide sequence of an influenza virus, preferably influenza A virus at at one or more positions corresponding to a position selected from nucleotide 1107 (P1), nucleotide 1275 (P2), nucleotide 1302 (P3), nucleotide 1404 (P4), nucleotide 1467 (P5), and nucleotide 1476 (P6) of SEQ ID No: 1, it is preferred that (i) the codon CCA is mutated to CCG, CCT or CCC; (ii) the codon CCG is mutated to CCA, CCT or CCC; (iii) the codon CCT is mutated to CCA, CCG or CCC; (iv) the codon CCC is mutated to CCA, CCG or CCT.

Moreover, in a preferred embodiment of the present invention, the method for obtaining a live, attenuated virus having a segmented genome further comprises the step of substituting one or more nucleotide(s) in a nucleotide sequence encoding NP from an influenza virus, preferably influenza A virus, corresponding to the nucleotide at a position selected from nucleotide 1107 (P1), nucleotide 1275 (P2), nucleotide 1302 (P3), nucleotide 1404 (P4), nucleotide 1467 (P5), and nucleotide 1476 (P6) of SEQ ID No: 1 by a synonymous nucleotide(s). As mentioned herein, any of the P1-P6 is the third position in a codon coding for proline. Accordingly, dependent on which nucleotide is present at the third position of a proline encoding codon in a NP encoding nucleotide sequence at a position corresponding to a position selected from nucleotide 1107 (P1), nucleotide 1275 (P2), nucleotide 1302 (P3), nucleotide 1404 (P4), nucleotide 1467 (P5), and nucleotide 1476 (P6) of SEQ ID No: 1 (i) the codon CCA is mutated to CCG, CCT or CCC; (ii) the codon CCG is mutated to CCA, CCT or CCC; (iii) the codon CCT is mutated to CCA, CCG or CCC; (iv) the codon CCC is mutated to CCA, CCG or CCT.

Nucleic acid molecules encoding the proteins of the Influenza A virus and the proteins of Influenza B virus, for example RNA segments of the respective virus, can be expressed using any suitable expression system. In some embodiments a suitable host cell is used. A suitable host cell is any cell that supports efficient replication of influenza virus, including mutant cells which express reduced or decreased levels of one or more sialic acids which are receptors for influenza virus. Viruses obtained by the methods can be made into a reassortant virus. Any cell, e.g., any avian or mammalian cell, such as a human, canine, bovine, equine, feline, swine, ovine, mink, e.g., MvLu1 cells, or non-human primate cell, including a mutant cell, which supports efficient replication of influenza virus can be employed to isolate and/or propagate influenza viruses. Isolated viruses can be used to prepare a reassortant virus, e.g., an attenuated virus. In one embodiment, a host cell for vaccine production is a cell found in avian eggs. In another embodiment, a host cell for vaccine production is a cell of a continuous mammalian or avian cell line or cell strain. Examples of suitable cell lines include, but are not limited to the Mardin-Darby Bovine Kidney (MDBK) cell line, the Madin-Darby Canine Kidney (MDCK) cell line, Vero cells (African green monkey kidney cells), the baby hamster kidney cell line BHK21-F, hamster kidney cell line HKCC and the human embryonic retinal cell line PER.C6® (Crucell Holland B.V.). Two further exemplary cell lines that may be suitable for efficient viral replication are human embryonic kidney HEK-293 cells or chicken fibroblasts DFI.

A suitable host cell is in some embodiments a cell of a WHO certified, or certifiable, continuous cell line. The requirements for certifying such cell lines include characterization with respect to at least one of genealogy, growth characteristics, immunological markers, virus susceptibility tumorigenicity and storage conditions, as well as by testing in animals, eggs, and cell culture. Such characterization is used to confirm that the cells used are free from detectable adventitious agents. In some countries, karyology may also be required. In addition, tumorigenicity is preferably tested in cells that are at the same passage level as those used for vaccine production. The virus may be purified by a process that has been shown to give consistent results, before vaccine production.

The terms “expression” and “expressed”, as used herein, are used in their broadest meaning, to signify that a sequence included in a nucleic acid molecule and encoding a peptide/protein is converted into its peptide/protein product. Thus, where the nucleic acid is DNA, expression refers to the transcription of a sequence of the DNA into RNA and the translation of the RNA into protein. Where the nucleic acid is RNA, expression may include the replication of this RNA into further RNA copies and/or the reverse transcription of the RNA into DNA and optionally the transcription of this DNA into further RNA molecule(s). In any case expression of RNA includes the translation of any of the RNA species provided/produced into protein. Hence, expression is performed by translation and includes one or more processes selected from the group consisting of transcription, reverse transcription and replication. Expression of the protein or peptide of the member of the plurality of peptides and/or proteins may be carried out using an in vitro expression system. Such an expression system may include a cell extract, typically from bacteria, rabbit reticulocytes or wheat germ. Many suitable systems are commercially available. The mixture of amino acids used may include synthetic amino acids if desired, to increase the possible number or variety of proteins produced in the library. This can be accomplished by charging tRNAs with artificial amino acids and using these tRNAs for the in vitro translation of the proteins to be selected. A nucleic acid molecule, such as DNA, is said to be “capable of expressing” a peptide/protein if it contains nucleotide sequences which contain transcriptional and translational regulatory information and such sequences are operably linked to nucleotide sequences which encode the polypeptide. A suitable embodiment for expression purposes is the use of a vector, in particular an expression vector. Thus, the present invention also provides a host cell transformed/transfected with an expression vector.

An expression vector, which may include one or more regulatory sequences and be capable of directing the expression of nucleic acids to which it is operably linked. An operable linkage is a linkage in which a coding nucleotide sequence of interest is linked to one or more regulatory sequence(s) such that expression of the nucleotide sequence sought to be expressed can be allowed. Thus, a regulatory sequence operably linked to a coding sequence is capable of effecting the expression of the coding sequence, for instance in an in vitro transcription/translation system or in a cell when the vector is introduced into the cell. A respective regulatory sequence need not be contiguous with the coding sequence, as long as it functions to direct the expression thereof. Thus, for example, intervening untranslated yet transcribed sequences may be present between a promoter sequence and the coding sequence and the promoter sequence can still be considered “operably linked” to the coding sequence.

The term “regulatory sequence” includes controllable transcriptional promoters, operators, enhancers, silencers, transcriptional terminators, 5′ and 3′ untranslated regions which interact with host cellular proteins to carry out transcription and translation and other elements that may control gene expression including initiation and termination codons. The regulatory sequences can be native (homologous), or can be foreign (heterologous) to the cell and/or the nucleotide sequence that is used. The precise nature of the regulatory sequences needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence or CAAT sequence. These regulatory sequences are generally individually selected for a certain embodiment, for example for a certain cell to be used. The skilled artisan will be aware that proper expression in a prokaryotic cell also requires the presence of a ribosome-binding site upstream of the gene sequence-encoding sequence.

Hence, in some embodiments the PA gene of an Influenza A virus as defined above, or the HA gene of an Influenza A virus as defined above, may be included in a vector, such as an expression vector. Likewise, a PB1 gene, a PB2 gene, a PA gene, a HA gene, a NP gene, a NA gene, a M1 gene, a BM2 gene, a NS1 gene, and/or a NS2 gene, of an Influenza B virus as defined above, may be included in a vector, such as an expression vector. A respective vector may in some embodiments include a 3′ and/or a 5′ non-coding sequence of an IAV or of an IBV, respectively. In some embodiments the PA gene and/or the HA gene of an IAV and/or the PB1 gene, the PB2 gene, the PA gene, the HA gene, the NP gene, the NA gene, the M1 gene, the BM2 gene, the NS1 gene, and/or the NS2 gene, of an IBV is/are operably linked to a promoter, for example RNA polymerase I promoter, RNA polymerase II promoter, RNA polymerase III promoter, T7 promoter and T3 promoter, such as a respective human promoter, e.g. a human RNA polymerase I promoter. In some embodiments the PA gene and/or the HA gene of an IAV and/or the PB1 gene, the PB2 gene, the PA gene, the HA gene, the NP gene, the NA gene, the M1 gene, the BM2 gene, the NS1 gene, and/or the NS2 gene, of an IBV is/are linked to a transcription termination sequence, for example one of a RNA polymerase I transcription termination sequence, RNA polymerase II transcription termination sequence, RNA polymerase III transcription termination sequence, and a ribozyme.

The present invention relates to a live attenuated influenza virus as described herein for use in the vaccination against influenza. Likewise, the present invention relates to a live attenuated influenza virus as described herein for use treatment and/or prevention of influenza.

Similarly, the present invention relates to a method of treatment and/or prevention of influenza comprising administering to a subject in need thereof a composition comprising a live attenuated influenza virus as described herein. Likewise, the present invention relates to a method of vaccinating against influenza comprising administering to a subject in need thereof a composition comprising a live attenuated influenza virus as described herein.

A live attenuated Influenza virus of the present invention may be used for the prophylactic and/or therapeutic treatment of viral infections, in particular influenza virus infections, i.e., it may be used for the treatment and/or prevention of influenza. They may be administered as known in the art, e.g. intravenously, subcutaneously, intramuscularly or, most preferably, intranasally. For such purposes the virus of the composition that includes the virus may be provided in a suitable injectable or inhalable form. A live attenuated Influenza virus of the present invention may in some embodiments be included in a device for applying the virus in an inhalable or injectable form to a subject. An influenza virus with a silent mutation disclosed herein and the vaccines made thereof may, however, also be used as vectors or shuttles to present heterologous antigens to the immune system, e.g. antigens of viral envelope proteins such as HIV, SARS coronavirus, Ebola, Herpes or hepatitis antigens.

A pharmaceutical composition that includes an Influenza virus of the present invention may be manufactured in a manner that is itself known, e.g., by means of conventional mixing, dissolving, granulating, dragée-making, levigating, emulsifying, encapsulating, entrapping or lyophilising processes. The composition may be an immunogenic composition such as a vaccine. The respective vaccine forming the main constituent of the vaccine composition of the invention may include a single influenza virus, or a combination of influenza viruses, for example, at least two or three influenza viruses, including one or more reassortant(s). The dosage of a live, attenuated virus vaccine for an animal such as a mammalian adult organism may be from about 10² to 10¹⁵, e.g., about 10³ to about 10¹², about 10³ to about 10¹⁰, about 10³ to about 10⁸, about 10⁵ to about 10⁸, about 10³ to about 10⁶, about 10⁴ to about 10⁸, about 10⁴ to about 10⁷, about 10⁴ to about 10⁶ or about 10⁴ to about 10⁵ plaque forming units (PFU)/kg, or any range or value therein. However, the dosage should be a safe and effective amount as determined by conventional methods, using existing vaccines as a starting point.

A pharmaceutical composition for use in accordance with the present invention may be formulated in conventional manner using one or more pharmacologically acceptable carriers that include excipients and auxiliaries, which facilitate processing of the virus into preparations that can be used pharmaceutically. Proper formulation is dependent upon the selected route of administration. A composition, including its components, is said to be “pharmacologically acceptable” if its administration can be tolerated by a recipient mammal or bird. Such an agent is said to be administered in a “therapeutically effective amount” if the amount administered is physiologically significant. A composition of the present invention is physiologically significant if its presence results in a detectable change in the physiology of a recipient patient, e.g., enhances at least one primary or secondary humoral or cellular immune response against at least one strain of an infectious influenza virus.

Certain embodiments of any of the instant immunization and therapeutic methods further comprise administering to the subject at least one adjuvant. An “adjuvant” shall mean any agent suitable for enhancing the immunogenicity of an antigen and boosting an immune response in a subject. Numerous adjuvants, including particulate adjuvants, suitable for use with both protein- and nucleic acid-based vaccines, and methods of combining adjuvants with antigens, are well known to those skilled in the art. Suitable adjuvants for nucleic acid based vaccines include, but are not limited to, Quil A, imiquimod, resiquimod, and interleukin-12 delivered in purified protein or nucleic acid form. Adjuvants suitable for use with protein immunization include, but are not limited to, alum, Freund's incomplete adjuvant (FIA), saponin, Quil A, and QS-21.

Exemplary routes of administration of a pharmaceutical composition of the invention include oral, transdermal, and parenteral delivery. Suitable routes of administration may, for example, include depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

As an illustrative example, for injection, a pharmaceutical composition according to the present invention may be formulated as an aqueous solution, for example in physiologically compatible buffers such as Hanks's solution, Ringer's solution, or physiological saline buffer. For oral administration, a respective pharmaceutical composition can be formulated readily by combining the virus with pharmaceutically acceptable carriers well known in the art. Such carriers enable a virus of the invention to be formulated as tablets, pills, lozenges, dragées, capsules, liquids, gels, syrups, slurries, suspensions and the like, for oral ingestion by a patient to be treated.

Pharmaceutical preparations for oral use can be obtained by adding a solid excipient, optionally grinding a resulting mixture, and processing the mixture of granules, after adding suitable auxiliaries, if desired, to obtain tablets or dragée cores. Suitable excipients are, in particular, fillers such as sugars, including lactose, glucose, sucrose, mannitol, or sorbitol; starches and derivatives thereof, such as, corn starch, dextrin and wheat starch, rice starch, potato starch, hydroxypropyl starch, wheat starch, gelatine, gum tragacanth, methyl cellulose, hydroxypropylmethyl-cellulose, sodium carboxymethylcellulose, and/or polyvinylpyrrolidone (PVP); cellulose preparations such as, for example, methylcellulose, carboxylmethylcellulose and hydroxypropylcellulose; inorganic compounds, such as sodium chloride, boric acid, calcium sulfate, calcium phosphate and precipitated calcium carbonate. If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of virus doses.

Suitable fluidizing agents include, but are not limited to, magnesium oxide, synthetic aluminium silicate, metasilicic acid, magnesium aluminium oxide, hydrous silicic acid, anhydrous silicic acid, talc, magnesium stearate, and kaolin. Suitable binding agents include, but are not limited to, polyethylene glycol, polyvinyl pyrrolidine, polyvinyl alcohol, gum arabic, tragacanth, sodium alginate, gelatine, and gluten. Suitable stabilisers include, but are not limited to, proteins, such as albumin, protamine, gelatine and globulin; and amino acids and salts thereof. Suitable thickeners include, but are not limited to, sucrose, glycerine, methylcellulose, and carboxymethylcellulose. Suitable pH adjusting agents include, but are not limited to, hydrochloric acid, sodium hydroxide, phosphates, citrates, and carbonates.

Pharmaceutical compositions that can be used orally include, but are not limited to, push-fit capsules made of gelatine, as well as soft, sealed capsules made of gelatine and a plasticiser, such as glycerol or sorbitol. The push-fit capsules may contain the attenuated virus in admixture with filler such as lactose, binders such as starches, and/or lubricants such as talc or magnesium stearate and, optionally, stabilisers. In soft capsules, the virus(es) may be dissolved or suspended in suitable liquids, such as fatty oils, liquid paraffin, or liquid polyethylene glycols. In addition, stabilisers may be added. All formulations for oral administration should be in dosages suitable for such administration.

For buccal administration, a respective pharmaceutical composition may take the form of tablets or lozenges formulated in conventional manner.

For administration by inhalation, a pharmaceutical composition for use according to the present invention may conveniently be delivered in the form of an aerosol spray presentation from pressurised packs or a nebuliser, with the use of a suitable propellant, e.g., dichlorodifluoromethane, trichlorofluoromethane, dichlorotetrafluoroethane, carbon dioxide or other suitable gas. In the case of a pressurised aerosol the dosage unit may be determined by providing a valve to deliver a metered amount. Capsules and cartridges of e. g. gelatine for use in an inhaler or insufflator may be formulated containing a powder mix of the virus and a suitable powder base such as lactose or starch.

A respective pharmaceutical composition may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the virus in water-soluble form. Additionally, suspensions of the virus may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran.

In some embodiments an active ingredient, such as a virus as described above, may be in powder form for constitution with a suitable vehicle, e.g., sterile pyrogen-free (SPF) water, before use.

A pharmaceutical composition according to the present invention may be administered by, for example, the oral, topical, dermal, ocular, intravenous, intraarticular, rectal, vaginal, inhalation, intranasal, sublingual or buccal route. Accordingly, the present invention also provides administering to an organism, generally a mammal or a bird, an Influenza virus as defined above, including a composition that includes a respective Influenza virus. Any cell may be used in the present method of the invention. As an illustrative example, a tumour cell may be used. Examples of suitable mammals include, but are not limited to, a mouse, a rat, a cow, a goat, a sheep, a pig, a dog, a cat, a horse, a guinea pig, a canine, a hamster, a mink, a seal, a whale, a camel, a chimpanzee, a rhesus monkey and a human. Examples of suitable birds include, but are not limited to, a turkey, a chicken, a goose, a duck, a teal, a mallard, a starling, a Northern pintail, a gull, a swan, a Guinea fowl or water fowl to name a few. Reports further indicate that the host range of influenza A virus may be expanding, so that it may be required to administer a virus of the invention to any further bird or mammal. As explained above, an Influenza B virus almost exclusively infects humans, whereas Influenza A virus infects a large variety of mammals, including domestic poultry.

Exemplary routes of administration of a respective pharmaceutical composition with an attenuated virus include oral, transmucosal, intranasal and parenteral delivery (see also above), including intramuscular, subcutaneous, intravenous, intramedullary injections, as well as intrathecal, direct intraventricular, intraperitoneal, intranasal, or intraocular injections. The amount of virus that is used can be chosen by the skilled person having regard to the usual factors.

In conjunction with a composition, e.g. a vaccine composition, the present invention also provides a method of inducing a protective immune response to an influenza infection in an animal, such as a bird or a mammal, including a human. The method includes administering to the animal the attenuated influenza virus, which is included in the respective composition. As noted above, in one embodiment, the vaccine composition is administered mucosally. In another embodiment, the vaccine composition is administered conjointly with an adjuvant.

Further provided is a method to prepare a live, attenuated recombinant influenza virus. Some embodiments of this method include contacting a host cell with one or more vectors that include(s) the IAV NP-gene and/or PA-gene as defined above. As explained above, an IAV NP-gene and/or PA-gene according to the invention includes a silent mutation, which per se does not result in a mutant NP and PA protein, respectively. In one embodiment, the mutant NS1 has one or more additional amino acid residues at the C-terminus. In one embodiment, the vector with the IAV NP-gene and/or the PA-gene is an RNA vector. In one embodiment, the vector with the IAV NP gene and/or the PA gene is an DNA vector, which is being translated into RNA. In one embodiment, the IAV NP gene and the IAV PA gene are based on same IAV virus strain, e.g. obtained by mutagenesis of a nucleic acid of the same virus strain, such as the same virus isolate. In one embodiment, the IAV NP gene and the IAV PA-gene are based on different IAV influenza virus strains.

Further the method includes contacting the host cell with a plurality of vectors that include the remaining IAV genes that are necessary to form an infectious IAV virus. The further IAV genes are generally the genes encoding viral polymerase subunits polymerase basic proteins 1 and 2 (PB1 and PB2) and polymerase acidic protein (PA), the protein hemagglutinin (HA), the nucleoprotein (NP), neuraminidase (NA), the matrix proteins M1 and M2, the protein NS1, and the nuclear export protein (NEP), also termed NS2.

In some embodiments these remaining IAV genes are based on the same IAV virus strain, e.g. obtained from a nucleic acid of the same virus strain, such as the same virus isolate. In one embodiment the IAV virus strain from which these remaining IAV genes are obtained or on which they are based is the same as the virus strain from which the IAV NP-gene and/or PA-gene are obtained or on which they are based. In some embodiments, the IAV NP-gene and the IAV PA-gene are based on one or more IAV virus influenza virus strains that differ from those virus strains on which the remaining IAV genes are based or from which they are obtained. In one embodiment each of the remaining IAV genes is based on a different virus isolate or a different virus strain, or obtained from a different virus isolate or a different virus strain. The method may also include culturing the host cell. As an illustrative example, in a suitable serum-free culture medium MDCK cells may be cultIAVted, for instance as adherent cells, infected and further proliferated, for example over several days. The serum-free medium may in some embodiments include a plant hydrolysate, a lipid supplement, trace elements, and is fortified with one or more medium component selected from the group consisting of putrescine, amino acids, vitamins, fatty acids, and nucleosides. Further the method may include isolating infectious IAV from the host cell. Isolating the virus, for example from cell culture, may include a chromatography technique and/or membrane filtration, for example for clarification, buffer exchange or concentration purposes.

Some embodiments of the method of preparing a live, attenuated recombinant influenza virus include contacting a host cell with one or more vectors that include(s) the IBV PB1 gene, PB2 gene, PA gene, HA gene, NP gene, NA gene, M1 gene, BM2 gene, NS1 gene, and/or NS2 gene that includes a silent mutation. The above said with regard to the IAV, or a vector that includes the same, used in a method of the invention applies mutatis mutandis to an IBV gene. Thus the vector with the respective IBV gene may for instance be an RNA vector or a DNA vector. Likewise, the host cell is contacted with a plurality of vectors that include the remaining IBV genes that are necessary to form an infectious IBV virus. The host cell may also be cultured and infectious IBV be isolated from the host cell.

If desired, an influenza virus can be passaged at least once in the allantoic cavity of embryonated eggs, such as chicken eggs, in the presence of serum, to obtain serum-resistant virus.

In a further aspect, the present invention provides a method for identifying (a) nucleotide(s) within influenza virus RNA packaging signals in a gene segment that, when replaced by a synonymous mutation, result(s) in an attenuated influenza virus, said method comprising (a) comparing a plurality of nucleotide sequences of RNA packaging signals of a gene segment of an influenza virus by sequence alignment; (b) identifying (a) conserved nucleotide(s); (c) substituting said conserved nucleotide(s) by a synonymous nucleotide (i.e., introducing a synonymous mutation); and (d) determining whether an influenza virus containing said synonymous mutation at the position(s) corresponding to the respective position(s) within the RNA packaging signal of an influenza virus not containing said synonymous mutation is attenuated in comparison to the same influenza virus not containing said synonymous mutation within the respective RNA packaging signal.

In a preferred embodiment of said method, a conserved nucleotide is present in at least 60-90% (including 60, 70, 80 or 90%) of the nucleotide sequences of RNA packaging signals that are compared (aligned) with each other.

Also, the present invention envisages an influenza virus having one or more of the silent mutations introduced in the gene segment(s) in accordance with said method.

Finally, the present invention envisages a method for obtaining an attenuated virus with a segmented genome, said method comprising
(a) comparing a plurality of nucleotide sequences of RNA packaging signals of a gene segment of a virus with a segmented genome;
(b) identifying (a) conserved nucleotide(s) at the third position of a codon within a RNA packaging signal;
(c) substituting said conserved nucleotide(s) by (a) synonymous nucleotide(s) (i.e., introducing a synonymous mutation);
(d) producing a virus with a segmented genome comprising said synonymous nucleotide(s);
(e) determining whether a virus with a segmented genome containing said synonymous nucleotide(s) at the position(s) corresponding to the respective position(s) within the RNA packaging signal of a virus with a segmented genome not containing said synonymous nucleotide(s) is attenuated in comparison to the same virus with a segmented genome not containing said synonymous nucleotide(s) within the respective RNA packaging signal; and
(f) obtaining said attenuated virus with a segmented genome.

Also, the present invention envisages a virus with a segmented genome having one or more of the silent mutations introduced in the gene segment(s) in accordance with said method.

Viruses with a segmented genome which are envisaged by the present invention include viruses of the family orthomyxoviridae, bunyaviridae and arenaviridae. Orthomyxoviridae include Influenza A virus, Influenza B virus and Influenza C virus. Bunyaviridae include Bunyamwera virus, LaCrosse virus, California encephalitis virus, Rift-Valley-fever virus and hamtaviruses. Arenaviridae include Lymphocytic choriomeningitis virus (LCMV), Lassa virus, Juni virus (Argentine haemorrhagic fever).

All aspects, embodiments, definitions disclosed herein for influenza viruses also apply to the method for obtaining an attenuated virus with a segmented genome.

Additional objects, advantages, and features of this disclosure will become apparent to those skilled in the art upon examination of the following examples thereof, which are not intended to be limiting. Thus, it should be understood that although the present disclosure is specifically disclosed by exemplary embodiments and optional features, modification and variation of the disclosures embodied therein herein disclosed may be resorted to by those skilled in the art, and that such modifications and variations are considered to be within the scope of this disclosure.

### EXAMPLES

The Examples illustrate the invention and should not be construed as limiting the scope of the invention.

### Materials and Methods

### Example 1

### Identification of the Highly Conserved Regions in NP Gene Segment of IAV Crucial for Efficient Viral Replication

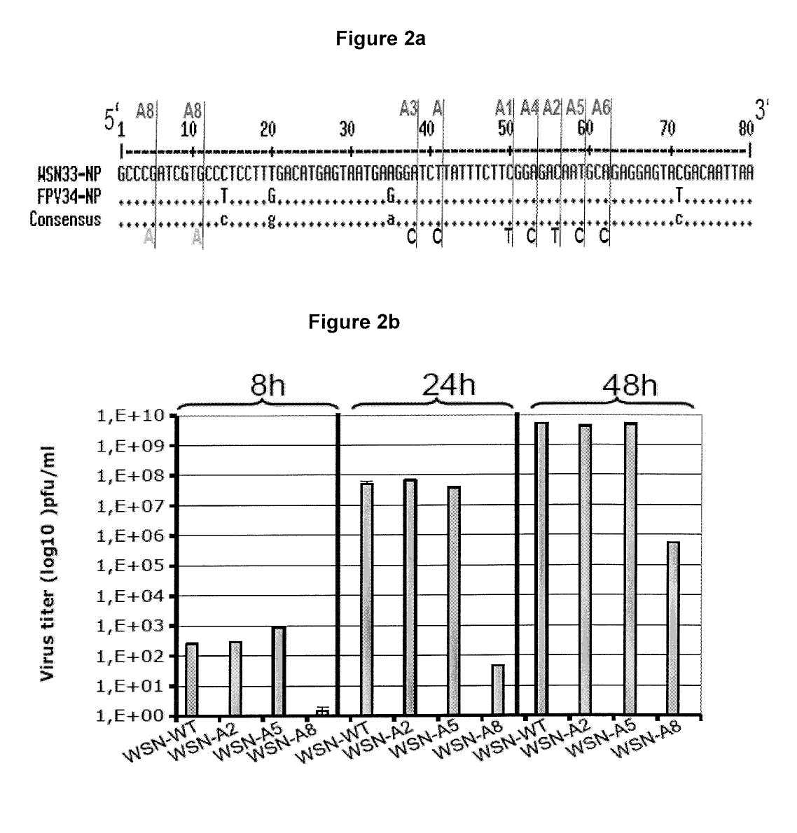

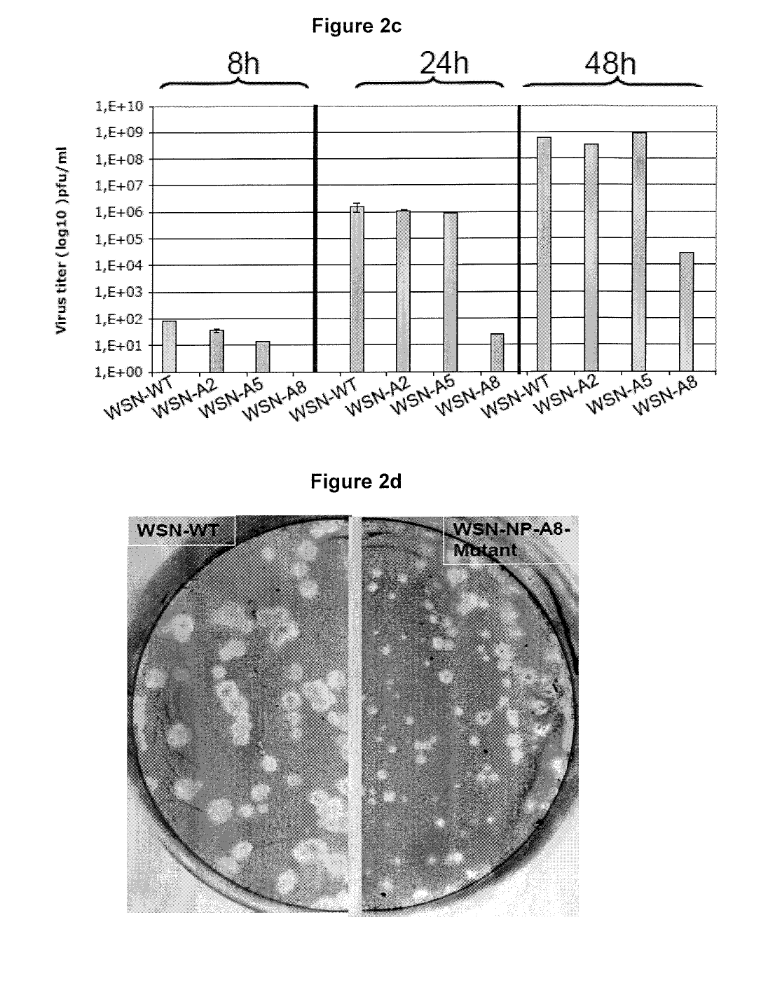

Excessive sequences comparisons of several hundred NP genes elucidated a highly conserved region within the ORF at the Tend of the cRNA, even exempt from the incorporation of silent mutations. This suggests that integrity of the RNA structure itself at this region is crucial for IAV replication, possibly for the specific incorporation of the NP-RNA into new forming virus particles. To recognize the possible essential sites of the NP gene segment for viral replication we have introduced silent mutations (from 1 to 9) at the 3^{rd} base of the corresponding codons in the pHW2000-WSN-NP plasmid by site directed mutagenesis method (FIG. 2a).

In order to analyze the importance of these conserved nucleotides for efficient virus replication, we created mutant viruses with silent mutations in the respective region of the NP gene of two different IAV strains (WSN/33 (H1N1), FPV/34 (H7N1)). First we studied the impact of the NP mutants of WSN viruses by virus growth curves on MDCK cells. Therefore, the WSN-NP-A2, -A5 and A8 mutant viruses with NP gene carrying 3 and 6 or 9-mutations, respectively, were compared with the WT virus for their replication capacity after infection of MDCK cells at two different MOI (0.01 or 0.001 MOI). Significant reduced virus titers were obtained for the A8 mutant virus which contains 9 silent mutations in comparison to the WT virus. (FIGS. 2b and 2c). This result demonstrates a growth disadvantage of IAV carrying multiple silent mutations within the NP gene. In addition to reduced virus titers, the NP-A8 mutant virus formed significant smaller plaques than the WT virus. (FIG. 2d).

### Example 2

### WSN NP-A8 Mutant Virus is Replication Attenuated and Safe in Mice

To determine the MLD₅₀ of the WSN-WT virus, mouse was infected with 1×10e3, lx 10e4, and 1×10e5 pfu doses of WT virus, respectively. MLD₅₀ was 10e4.1 pfu for WT virus. Then we started the analysis of the mouse pathogenicity to test a possible attenuation role of IAV NP gene with “silent” mutations. Three days after infection WT infected mice were already 20% deficient in their body weight.

While all this group mice, which infected with WT died by day 8 after inoculation, mice, which infected with the NP mutant virus showed a milder reduction of weight (ca. 3%) (FIG. 3a) (black triangle) from day 2 until day 8 after infection and do not developed any apparent disease symptoms during the infection time. Also, all of this NP mutant virus infected mice survived (FIG. 3b) (black triangle). These results demonstrated that a strong attenuation was induced in mice that infected by NP-A8 mutant virus with “silent” mutations than WT virus. These mice survival experiments were repeated 3 times. A strong attenuation was induced in mice which were infected a virus with silent mutated NP gene.

### Example 3

### Vaccination of Mice with A8 NP Virus Results in Complete Protection from Challenge Infection with Lethal Doses of WSN-WT, and with a New Swine Origin Pandemic 2009 H1N1 Viruses

To get more immunologic information about the genetically homolog protection or about broadly protective immunity against influenza A virus we did challenge analyses after infection with genetically apart IAV viruses. After a 45 days recover period with the WSN-A8 virus “immunized” mice were challenged with ca. 100 fold MLD50 (1×10e6 pfu) of the WSN-WT virus and with approx. 10 fold MLD (5×10e5 pfu) of the A/Hamburg/4/2009 v(H1N1) virus, respectively. This new swine origin pandemic H1N1 virus, which was isolated from a sick person in Hamburg during the human swine flu outbreak of 2009 is adapted to the Balb/c mouse by serial passaging in mice lung. As a control, mock-infected naïve mice were challenged with the same lethal dose of these viruses, respectively. All NP-A8 virus immunized mice, which were challenged with WSN-WT survived, almost no loss of body weight ((FIGS. 4a and 4b) white squares) was detectible in contrast to mock infected mice (mock group 1), which died by day 7 after WT virus infection. While all mock control mice (mock group 2) died by day 6 after inoculation, the NP-A8 virus immunized and A/Hamburg/4/2009 v(H1N1) virus challenged mice, weakly reduced their body weight until 2 days p.i. (post infection) without any apparent disease symptom development and then completely recovered within two weeks ((FIG. 4a) black triangles). Also, none of this new swine origin pandemic H1N1 virus infected mice died ((FIG. 4b). black squares). To measure the protective antibodies against the HA protein, 21 days after challenge infection serum from NP mutant virus infected mice was collected. A hemagglutination inhibition (HI) assay was performed with the collected 5 sera. In all cases the HI-titers were in a range from 256 to 512 (Table 1).

TABLE 1
Specific humoral immune response against the WSN virus but not against
the A/Hamburg/4/2009 v(H1N1) virus. A hemagglutination inhibition (HI)
assay was performed with mouse serum collected 21 days after challenge
infection with WSN-WT virus and A/Hamburg/4/2009 v(H1N1) virus
(n = 5). HI-titers were in a range from 256 to 512. As control serum
was tested for FPV/34/Rostock (H7N1) virus.

| | Challenged mice sera | | | | |
|---|---|---|---|---|---|
| | 1. | 2. | 3. | 4. | 5. |
| Virus dilution: | (1:256) | (1:512) | (1:512) | (1:512) | (1:512) |
| A/WSN/33-WT (H1N1) | + | + | + | + | + |
| (HA-titre 1:64) | | | | | |
| A/FPV/Rostock/34 | − | − | − | − | − |
| (H7N1) (HA-titre 1:16) | | | | | |
| A/Hamburg/4/2009 | − | − | − | − | − |
| v(H1N1) (HA-titre 1:64) | | | | | |

Note:
+ detected HI-titre in chamber;
− negative HI-titre

These results demonstrated that a specific humoral immune response against WSN was induced by the infection with the WSN-NP-A8 virus. HI-titre does detectable neither in FPV/34/Rostock (H7N1) virus nor in naive mock control mice serum. The FPV/34/Rostock (H7N1) virus was used as a negative control virus. To compare the pathogenicity for egg embryos and the growth capacity in embryonated eggs of WT and NP-A8 mutant virus each 10 eggs were infected with 3,5×10e5 pfu doses, respectively. After 4 days WT virus infected egg embryos died while the mutant virus survived and virus titers were grow until 10e 7 pfu.

### Example 4

### WSN NP-A8 Mutant Virus Replication is Significant Low in Mice Lung and the Packaging Level of vRNA Segment 5, and 3 of this Virus is Significantly Reduced than WT Virus

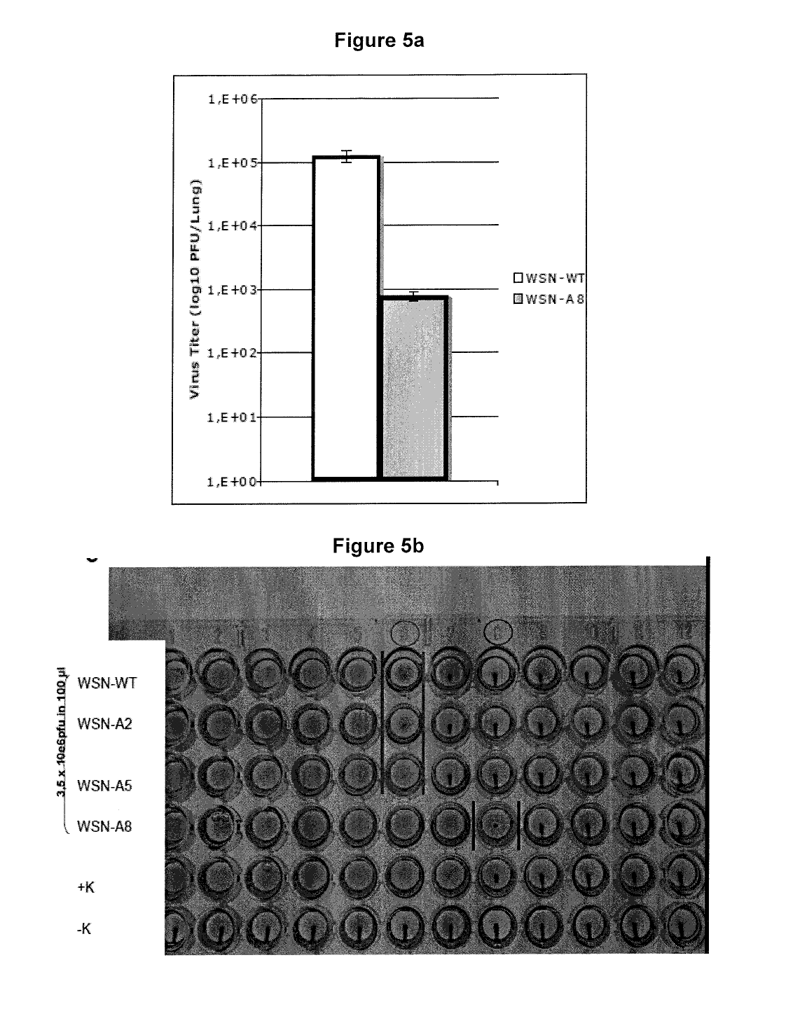

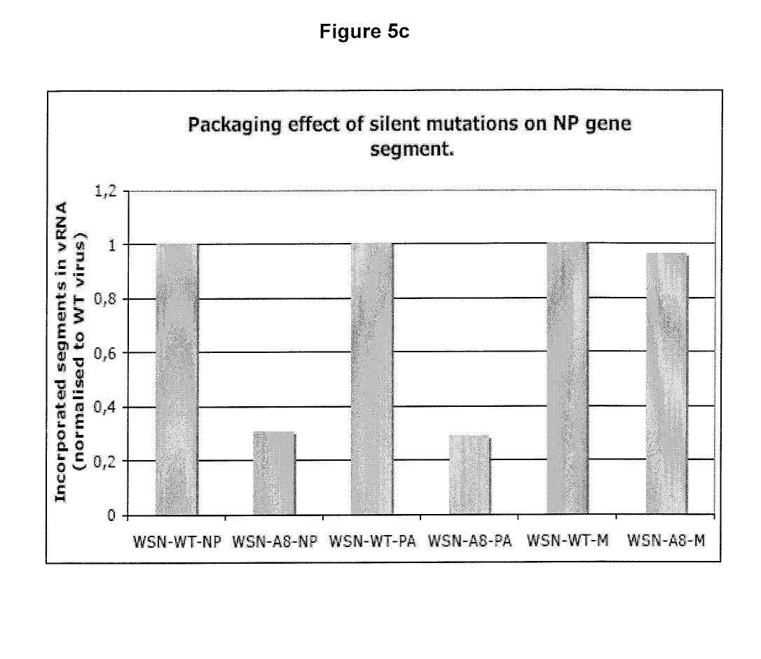

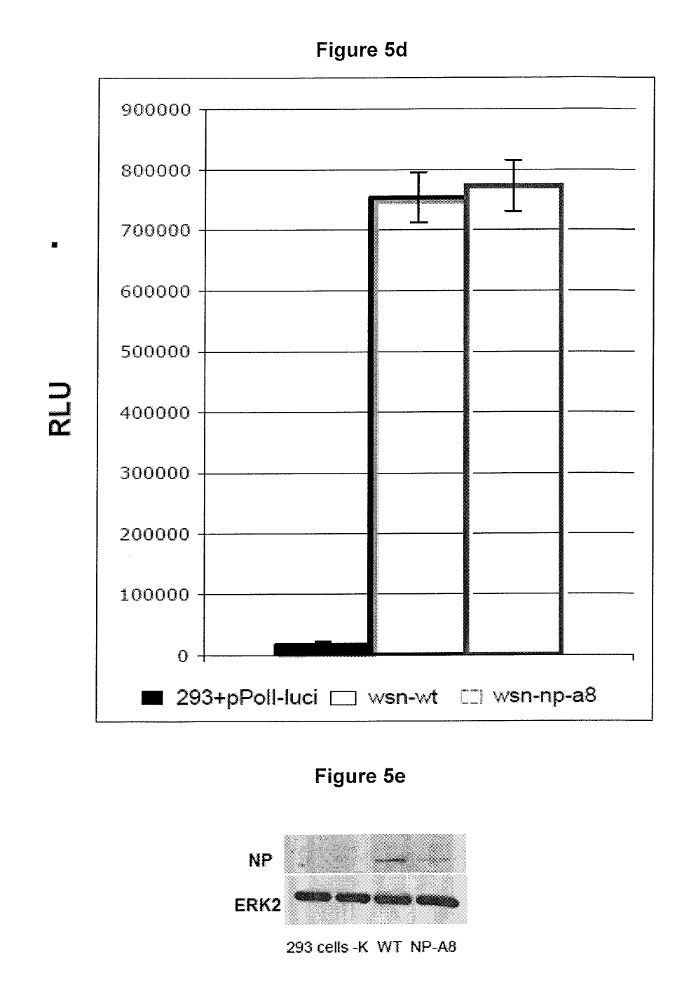

To determine the lung virus titers 3 mice were either infected with 1×10e5 pfu WSN-WT or WSN NP-A8 virus and after 3 days p.i. all infected mice were euthanized. Virus titers were detected from total lung homogenate by standard plaque assay in MDCK cells. The amount of infectious particles of WSN-WT virus in mouse lung was significantly higher than WSN NP-A8 virus (FIG. 5a). Interestingly 2 times more HA-titer of total virus particles was detectible in the mutant virus with 9 silent mutations than WT virus and other mutant viruses with few mutations (FIG. 5b). This result illustrates that the introduction of the 9 silent mutations within the NP led to the creation of more not infectious virus particles possibly due to NP segment packaging defects. To determine the exact mechanism of the WSN-A8 mutant virus replication defects, we tested the amount of packaged vRNA segments. In FIG. 5c the results of Real-Time-PCR analysis for both, the silent mutated segment 5 (NP) and not mutated segments (PA, and M) are shown, because previously a 2-fold lower incorporation of these segments after site directed mutations on the segment 5 of PR8 virus was found (Hutchinson et al. (2009), Vaccine 27:6270-6275. The incorporation level of the silent mutated segment 5 (NP) and non-mutated segment 2 (PA) was significantly approx. 3.5 fold reduced compared to WSN-WT virus. In contrast to these segments, the segment 7 (M) of both viruses was packaged almost equal (FIG. 5c). Shown is one representative similar result of three independent experiments. Taken these results together, we identified additional highly conserved key codons in the 5′ end of the vRNA of the NP gene, which are critical for the packaging of segment 5 and 3 as well as for the replication of influenza A virus. Finally we checked the polymerase activity of the WT and NP mutant mini-genome constructs by reporter-gene assay. 293 cells were transfected with the 4 plasmids (so called mini genome RNP constructs) and the Luciferase reporter-gene construct. Both NP-WT and NP-A8 mutant mini genome shown a almost identical level of enzymatic activity. But the NP protein expression of WSN-WT virus was stronger than mutant virus analysed by western blot using these 293 cell lysates (FIG. 5d). Eventually, this result argue for the assumption of an altered speed of protein expression because of different codon usage of silent mutated WSN-NP plasmid construct [21]. Obviously, WSN-NP-A8 virus, whose NP gene silent mutated in packaging region of the cRNA Tend without change existing amino acids show not only a effect for the some segment incorporation into virion but a altered intensity of NP protein expression. These molecular modifications for the NP gene of WSN virus lead to strong attenuation of WSN virus in mice. This attenuated WSN-NP-A8 virus could be used as a broad range live attenuated influenza virus vaccine candidate because of effective cross-protection against new swine origin pandemic 2009 (H1N1) virus.

Materials ands Methods

Cells, Virus and Plasmids.

Madin-Darby canine kidney (MDCK) cells were maintained in minimal essential medium (MEM) supplemented with 10% heat inactivated fetal bovine serum (FBS) and antibiotics. Human embryonic kidney (HEK293) cells were grown in DMEM medium supplemented with 10% heat inactivated FBS and antibiotics. For infection cells were washed with PBS incubated with virus at the indicated multiplicities of infection diluted in PBS/BA (PBS containing 0.2% bovine serum albumin (BSA), 1 mM MgCl₂, 0.9 mM CaCl₂, 100 U ml⁻¹ penicillin and 0.1 mg ml⁻¹ streptomycin) for 30 min at 37° C. The inoculum was aspirated and cells were incubated with either MEM or DMEM containing 0.2% BSA and antibiotics. At the given points in time supernatants were collected to assess the number of infections particles by standard plaque assays.

Generation of Recombinant Influenza Viruses

A set of plasmids allowing the rescue of the recombinant influenza virus strain A/WSN/33 and A/FPV/Rostock/34 was used for generating all NP gene mutants. The reverse genetics system includes eight influenza virus RNA-coding transcription plasmids as described elsewhere (Hoffmann et al. (2000), Proc Natl Acad Sci 97:6108-6113; Wagner et al. (2005), J Virol 79:6449-6458. To create the NP gene silent mutant viruses site-directed mutations were introduced into the NP gene cDNA of recombinant WSN/33 and A/FPV/Rostock/34 using the Quickchange mutagenesis kit (Stratagene). All mutations were chosen to not affect the open reading frame of the NP gene, respectively.

In order to generate the recombinant viruses, 1 μg each of the eight plasmids was transfected into HEK293 cells by using Lipofectamine 2000 (Invitrogen) as described by Hoffmann et al. (cited above). Briefly, twenty-four hours post transfection fresh DMEM (100 U ml-1 penicillin, and 0.1 mg ml-1 streptomycin, 0.5% heat inactivated FBS and 0.2% BSA media was added. After 24 h incubation the supernatant was removed and used for infection of new MDCK cells. After 3 days incubation the supernatant was harvested and the virus titer was determined on MDCK cells by standard plaque assays. Virus plaques were visualized by staining with neutral red and virus titers were indicated as PFU/ml. The NP gene of recombinant wild type and mutant viruses were sequenced after reverse transcription-PCR amplification from infected cells to verify the presence and propriety of the desired mutations.

RNA Isolation, Reverse Transcription, Quantitative Real-Time PCR, and RNA Packaging Analysis.

vRNA from virus pellets (1-3 ml MDCK supernatant or egg virus stocks) after ultracentrifugation (43000 rpm, 45 min, TLA 100.4 Rotor, Beckman) was isolated using the RNeasy Mini Kit from Qiagen, or using the High Pure Viral RNA Kit (Roche Applied Science, Mannheim, Germany) according to manufacturer's instructions, respectively. To synthesize cDNA 0,1 μg of total vRNA were reverse transcribed using 0.1 μg random primer and 200 U Revert Aid™ Premium Reverse Transcriptase (Fermentas, St. Leon-Rot, Germany) or using StrataScript® QPCR cDNA Synthesis Kit (Stratagene, USA) according to manufacturer's instructions, respectively. The following primers are used for the site-directed mutations of NP gene of WSN/33 virus. Underlined bold letters indicates the synonym mutations.

IAV5s_As
(SEQ ID NO: 25)
GTAATGAAGGATCCTATTTCTTCGGAG
IAV5s_Aas
(SEQ ID NO: 26)
CTCCGAAGAAATAGGATCCTTCATTAC
IAV5s_A1s
(SEQ ID NO: 27)
GATCCTATTTCTTTGGAGACAATGCAG
IAV5s_A1as
(SEQ ID NO: 28)
CTGCATTGTCTCCAAAGAAATAGGATC
IAV5s_A2s
(SEQ ID NO: 29)
TTTCTTTGGAGATAATGCAGAGGAG
IAV5s_A2as
(SEQ ID NO: 30)
CTCCTCTGCATTATCTCCAAAGAAA
Np1500_A3s
(SEQ ID NO: 31)
TGAGTAATGAAGGCTCCTATTTCTTTG
Np1500_A3as
(SEQ ID NO: 32)
CAAAGAAATAGGAGCCTTCATTACTCA
Np1515_A4s
(SEQ ID NO: 33)
CTATTTCTTTGGCGATAATGCAGAG
Np1515_A4as
(SEQ ID NO: 34)
CTCTGCATTATCGCCAAAGAAATAG
Np1521_A5s
(SEQ ID NO: 35)
TTTGGCGATAACGCAGAGGAGTA
Np1521_A5as
(SEQ ID NO: 36)
TACTCCTCTGCGTTATCGCCAAA
NP1524-A6s
(SEQ ID NO: 37)
TATTTCTTTGGCGATAACGCCGAGGAGTACGACAATTAAAG
NP1524-A6as
(SEQ ID NO: 38)
CTTTAATTGTCGTACTCCTCGGCGTTATCGCCAAAGAAATA
NP2x-s
(SEQ ID NO: 39)
AAAAGGCAACGAGCCCAATCGTACCCTCCTTTGACATGAGTAATG
NP2x-As
(SEQ ID NO: 40)
CATTACTCATGTCAAAGGAGGGTACGATTGGGCTCGTTGCCTTTT

For quantification of cDNA real-time PCR was performed and calculated using the Roche Light Cycler® 480 III (F. Hoffmann-La Roche Ltd., Basel, Switzerland) using delivered instrument special protocol and by default program. To analyse the packaging effect of silent mutated NP gene is used the TaqMan probes of Universal ProbeLibrary Set (Roche Applied Science, Mannheim, Germany). Used TaqMan probe sequences are as follows:

1. for the NP gene Universal ProbeLibrary probe (UPL) #65, (cat.no. 04688643001). Sense primer (5′-gcggggaaagatcctaagaa) (SEQ ID NO:41) and Antisense primer (5′-tccactttccatctactctcctg) (SEQ ID NO:42)
2. for the M gene UPL #159, cat.no. 04694465001 Sense primer (5′-cctggtatgtgcaacctgtg) (SEQ ID NO:43) and Antisense primer (5′-tgtcaccatttgcctatgaga) (SEQ ID NO:44)
3. for the PA gene UPL#7, cat.no. 04685059001 Sense primer (5′-ctgacccaagacttgaaccac) (SEQ ID NO:45) and Antisense primer (5′-agcatatctcctatctcaagaacaca) (SEQ ID NO:46). Luciferase-reporter gene assays using pPoll-luc construct were carried out as described in Ludwig et al. (2001), J Biol Chem 276:10990-10998. The following constructs were used: pHW2000-WSN-PB2, pHW2000-WSN-PB1, pHW2000-WSN-PA, pHW2000-WSN-NP, and pHW2000-WSN-NP-A8 (described in Hoffmann et al. (2000) Proc Natl Acad Sci USA 97:6108-6113 in material and methods).

In-Vivo Experiments

### Infection of Mice

The MLD₅₀ (mouse lethal dose 50%) was determined by the method of Reed and Muench (1938), Am J Epidemiology 27:493-497 The 7-9 week old Balbc mice were anaesthetized by intraperitoneal (i.p.) injection of 200-250 μl ketamine-rompun solution (2% rompun solution and a 10% ketamine solution were mixed at the ratio of 1:10), weighed and infected by intranasal instillation of 25 μl/nostril of recombinant influenza virus (A/WSN/33 virus strain (H1N1)) (WSN WT) or containing silent mutated NP (WSN-A8). A total amount of 1×10³, 1×10⁴, 1×10⁵, and 1×10⁶ PFU per animal was instilled, respectively. Body weight or other signs of disease was recorded daily during the course of infection. 21 or 45 days after infection, the mouse was challenged with 1×10⁶ pfu/50 μl WSN-WT (ca. 100-fold MLD₅₀) Mouse experiments were repeated three times with at least 4 animals per each group. In vivo experiments were performed in strict accordance with the German regulations of the Society for Laboratory Animal Science (GV-SOLAS) and the European Health Law of the Federation of Laboratory Animal Science Associations (FELASA). All experimental procedures were performed in a Biosafety level 2 facility.

Mouse survival analysis (Kaplan-Meier plots) were conducted by WinStat® and MLD₅₀ calculation was performed as described by Reed and Muench (1938), Am J Epidemiology 27:493-497.

Hemaaglutination Inhibition (HAI) Assay

Blood serum of vaccinated mice was collected 3 weeks after infection. HAI assays were performed in V-bottomed microtiter plates using 50 μl of fresh 0.5-1.0% suspensions of chicken red blood cells in PBS. 100 μl serum from vaccinated mice was added and serially diluted in PBS. Then, 50 μl of a 1:64 virus dilution (ca. 3.5×10⁵ pfu/well) was added to the serum. After 30 min incubation at room temperature (20-22° C.) 50 μl of chicken erythrocytes were added to the wells and were analyzed following 1 h incubation period on 4° C. An inhibition of the hemagglutination was indicated, when red blood cells precipitated to the bottom of the plate, while red blood cells incubated with influenza virus or control serum showed a diffuse distribution on the microtiter plates illustrating an positive agglutination of erythrocytes. The HAI titers were given as reciprocal of the highest dilution causing detectable inhibition of hemagglutination.

1. A method for obtaining a live, attenuated virus having a segmented genome, said method comprising
(a) comparing a plurality of nucleotide sequences of RNA packaging signals of a gene segment of virus having a segmented genome;
(b) identifying (a) conserved nucleotide(s) at the third position of a codon within an RNA packaging signal;
(c) substituting said conserved nucleotide(s) by (a) synonymous nucleotide(s) (i.e., introducing a synonymous mutation);
(d) producing a virus having a segmented genome comprising said synonymous nucleotide(s);
(e) determining whether a virus having a segmented genome containing said synonymous nucleotide(s) at the position(s) corresponding to the respective position(s) within the RNA packaging signal of a virus having a segmented genome not containing said synonymous nucleotide(s) is attenuated in comparison to the same a virus having a segmented genome not containing said synonymous nucleotide(s) within the respective RNA packaging signal; and
(f) obtaining said live, attenuated virus having a segmented genome.

2. The method of claim 1, wherein the virus having a segmented genome is a virus of the family orthomyxoviridae, bunyaviridae or arenaviridae.

3. The method of claim 1 or 2, wherein the virus having a segmented genome is influenza A virus.

4. The method of claim 1, wherein the nucleotide sequence(s) of RNA packaging signals of a gene segment is/are from influenza a virus.

5. The method of claim 4, wherein said gene segment is from the influenza virus NP, PA, PB1, PB2, HA, NA, M, NS, BM2, or NS-2 gene.

6. The method of claim 1, wherein the RNA packaging signal comprises all nucleotides of the 5′ non-coding region and 9-250 nucleotides adjacent (5′→3′) to said nucleotides of the 5′ non-coding region of a gene segment and/or comprises all nucleotides of the 3′ non-coding region and 20-230 (including 20, 21, 22, 23, 24, 25, 30, 35, 40, 45, 50, 60, 70, 80, 90, 100, 110, 120, 130, 140, 150, 160, 170, 180, 190, 200, 210, 220) nucleotides adjacent (3′→5′) to said nucleotides of the 3′ non-coding region of a gene segment.

7. The method of claim 4, wherein the RNA packaging signals of a gene segment from influenza virus comprises a plurality comprising at least 2, at least 10, at least 20, or at least 50 nucleotide sequences of RNA packaging signals.

8. The method of claim 7, wherein a nucleotide is conserved, if it is present in at least 60% of the nucleotide sequences that are compared.

9. An attenuated influenza virus obtainable by the method of claim 1.

10. The attenuated influenza virus of claim 9 which is an influenza A virus.

11. The attenuated influenza virus of claim 9 or 10, having a silent mutation at one or more positions corresponding to a position selected from nucleotide 1107 (P1), nucleotide 1275 (P2), nucleotide 1302 (P3), nucleotide 1404 (P4), nucleotide 1467 (P5), and nucleotide 1476 (P6) of SEQ ID No: 1.

12. A composition comprising the attenuated influenza virus of claim 9, and a pharmaceutically acceptable carrier.

13. The composition of claim 12, wherein said influenza virus contains a nucleoprotein (NP) gene having a silent mutation at one or more positions corresponding to a position selected from nucleotide 1467 (NP-A7), nucleotide 1473 (NP-A8), nucleotide 1500 (NP-A3), nucleotide 1503 (NP-A), nucleotide 1512 (NP-A1), nucleotide 1515 (NP-A4), nucleotide 1518 (NP-A2), nucleotide 1521 (NP-A5), and nucleotide 1524 (NP-A6) of SEQ ID No: 1.

14. The composition of claim 13, the NP gene having a silent mutation of at least 2, 3, 4, 5, 6, 7, 8, or 9 of the positions corresponding to nucleotide 1467, nucleotide 1473, nucleotide 1500, nucleotide 1503, nucleotide 1512, nucleotide 1515, nucleotide 1518, nucleotide 1521 and nucleotide 1524 of SEQ ID No: 1.

15. The composition of claim 13, being a vaccine composition.

16. The composition of claim 13, wherein the composition is formulated for use in immunizing a mammal or a bird.

17. The composition of claim 16, wherein the mammal is a human.

18. The composition of claim 16 or 17, wherein the mammal is immunocompromised.

19. The composition of claim 13, wherein the NP gene encodes an NP polypeptide.

20. The composition of claim 13, wherein the influenza virus further contains PA gene, the PA gene having a silent mutation at one or more positions corresponding to a position selected from nucleotide 2100 and nucleotide 2103 of SEQ ID No: 3.

21. The composition of claim 20, wherein the PA gene has a silent mutation at both positions defined in claim 20.

22. The composition of claim 20, wherein the gene PA gene encodes a PA polypeptide.

23. The composition of claim 9, the composition conferring to a serum sample from a mammal or from a bird, to which mammal or bird there has been administered at least one dose of about 104 to about 105 PFU/kg of the attenuated influenza virus, a hemagglutinin inhibition (HI) titer of at least about 1:520, when tested against the same influenza virus not having said one or more silent mutations.

24. The composition of claim 23, wherein the mammal is a dog, a cat, a rat, a rabbit, a pig, a goat, a mouse or a horse.

25. The composition of claim 24, wherein the bird is a chicken, a goose or a duck.

26. The composition of claim 13, said composition conferring protection against a 10-100-fold lethal dose of an IAV corresponding to the IAV of any one of claims 1-7, the IAV not having said one or more silent mutations to an animal that has been administered at least one dose of about 104 to about 105 PFU/kg of the IAV virus of any one of claims 1-7.

27. An influenza A virus (IAV) PA gene comprising a silent mutation at one or more positions corresponding to nucleotide 2100 and nucleotide 2103 of SEQ ID No: 3.

28. The IAV PA gene of claim 27, wherein the NP gene is comprised in a vector.

29. The IAV PA gene of claim 28, wherein the vector further comprises a 3′ and a 5′ noncoding sequence of an IAV.

30. The IAV PA gene of any one of claims 27 to 29, said PA gene being operably linked to a promoter.

31. The IAV PA gene of claim 30, wherein the promoter is a promoter selected from the group consisting of RNA polymerase I promoter, RNA polymerase II promoter, RNA polymerase III promoter, T7 promoter and T3 promoter.

32. The IAV PA gene of claim 27, wherein the PA gene is linked to a transcription termination sequence.

33. The IAV PA gene of claim 32, wherein the transcription termination sequence is selected from the group consisting of RNA polymerase I transcription termination sequence, RNA polymerase II transcription termination sequence, RNA polymerase III transcription termination sequence, and a ribozyme.

34. A host cell comprising a vector, the vector comprising the IAV PA gene as defined in claim 27.

35. A method for the preparation of a live, attenuated IAV comprising
(a) introducing into a host cell
(i) a vector comprising the IAV PA gene of claim 27; and
(ii) a plurality of vectors comprising the remaining IAV genes required to form an infectious IAV; and
(b) isolating infectious IAV from said host cell.

36. A method for the preparation of a live, attenuated IAV comprising
(a) culturing the host cell of claim 34; and
(b) isolating infectious IAV from said host cell.

37. The method of claim 35, wherein the remaining IAV genes are a PB1 gene, a PB2 gene, a HA gene, a NA gene, a NS1 gene, a NS2 gene, a M1 gene, a M2 gene, and a NP gene.

38. The method of claim 35 further comprising
(c) formulating said infectious IAV with a pharmaceutically acceptable carrier.

39. A live, attenuated IAV comprising an IAV PA gene of claim 27.

40. A live, attenuated IAV obtainable by the method claim 35.

41. A vaccine composition comprising a live, attenuated IAV of claim 39 or 40 and a pharmaceutically acceptable carrier.

42. The live, attenuated IAV of claim 39 or 40 formulated for use in immunizing a mammal or a bird.

43. The live, attenuated IAV of claim 42, wherein the mammal is a human.
